(19) United States
(12) Patent Application Publication
(10) Pub. No.: US 2024/0208956 A1
(43) Pub. Date: Jun. 27, 2024

(54) 1,3,4-OXADIAZOLE THIOCARBONYL COMPOUNDS AS HISTONE DEACETYLASE 6 INHIBITOR, AND PHARMACEUTICAL COMPOSITION COMPRISING THE SAME

(71) Applicant: CHONG KUN DANG PHARMACEUTICAL CORP., Seoul (KR)

(72) Inventors: Chang Sik Lee, Yongin (KR); Jung Taek Oh, Yongin (KR); Hyeseung Song, Yongin (KR); Hyunjin Michael Kim, Yongin (KR)

(21) Appl. No.: 18/286,037

(22) PCT Filed: Apr. 7, 2022
(86) PCT No.: PCT/IB22/53253

§ 371 Date: Oct. 6, 2023

(30) Foreign Application Priority Data
Apr. 8, 2021 (KR) 10-2021-0046134

(51) Int. Cl. C07D 413/14; A61K 31/4245; A61K 31/438; A61K 31/4439; A61K 31/496; A61K 31/4995; A61K 31/5377; C07D 413/12; C07D 417/14; C07D 471/10; C07D 487/08; C07D 487/10; C07D 491/107

(52) U.S. Cl. CPC C07D 413/14; A61K 31/4245; A61K 31/438; A61K 31/4439; A61K 31/496; A61K 31/4995; A61K 31/5377; C07D 413/12; C07D 417/14; C07D 471/10; C07D 487/08; C07D 487/10; C07D 491/107

(57) ABSTRACT

The present invention relates to a novel 1,3,4-oxadiazole thiocarbonyl compound having a histone deacetylase 6 (HDAC6) inhibitory activity, stereoisomers thereof, pharmaceutically acceptable salts thereof, a use thereof for preparing a medicament, a pharmaceutical composition containing the same, a therapeutic method using the composition, and a preparation method thereof, wherein a novel compound having a selective HDAC6 inhibitory activity is represented by formula I.

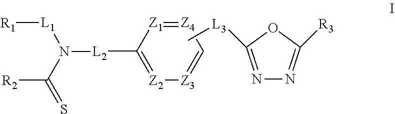

### TECHNICAL FIELD

The present invention relates to 1,3,4-oxadiazole thiocarbonyl compounds having a histone deacetylase 6 (HDAC6) inhibitory activity, stereoisomers thereof, pharmaceutically acceptable salts thereof; use thereof, use thereof for preparing a therapeutic drug, a method of treating diseases using the same; a pharmaceutical composition including the same; and a method for preparing the same.

### BACKGROUND ART

In cells, a post-translational modification such as acetylation serves as a very important regulatory module at the hub of biological processes and is also strictly controlled by several enzymes. As a core protein constituting chromatin, histone functions as an axis, around which DNAwinds, and thus helps a DNA condensation. Also, a balance between acetylation and deacetylation of histone plays a very important role in gene expression.

As an enzyme for removing an acetyl group from lysine residue of histone protein, which constitutes chromatin, histone deacetylase (HDAC) is known to be associated with gene silencing and induce a cell cycle arrest, angiogenic inhibition, immunoregulation, apoptosis, etc. (Hassig et al., Curr. Opin. Chem. Biol. 1, 300-308 (1997)). Also, it is reported that the inhibition of HDAC enzyme functions induces cancer cells into committing apoptosis for themselves by lowering the activity of cancer cell survival-related factors and activating cancer cell death-related factors in the body (Warrell et al., Natl. Cancer Inst. 90, 1621-1625 (1998)).

For humans, 18 HDACs are known and classified into four classes according to homology with yeast HDAC. In this case, eleven HDACs using zinc as a cofactor may be divided into three groups: Class I (HDAC1, 2, 3, 8), Class II (IIa: HDAC4, 5, 7, 9; IIb: HDAC6, 10) and Class IV (HDAC11). Further, seven HDACs of Class III (SIRT 1-7) use NAD+ as a cofactor instead of zinc (Bolden et al., Nat. Rev. Drug Discov. 5(9), 769-784 (2006)).

Various HDAC inhibitors are now in a preclinical or clinical development stage, but only non-selective HDAC inhibitors have been known as anti-cancer agents so far. Vorinostat (SAHA) and romidepsin (FK228) have obtained approval as a therapeutic agent for cutaneous T-cell lymphoma, while panobinostat (LBH-589) has won approval as a therapeutic agent for multiple myeloma. However, it is known that the non-selective H DAC inhibitors generally bring about side effects such as fatigue, nausea and the like at high doses (Piekarz et al., Pharmaceuticals 3, 2751-2767 (2010)). It is reported that the side effects are caused by the inhibition of class I HDACs. Due to the side effects, etc., the non-selective HDAC inhibitors have been subject to the restriction on drug development in other fields than an anticancer agent (Witt et al., Cancer Letters 277, 8-21 (2009)).

Meanwhile, it is reported that the selective inhibition of class II HDACs would not show toxicity, which has occurred in the inhibition of class I HDACs. In case of developing the selective HDAC inhibitors, it would be likely to solve side effects such as toxicity, etc., caused by the non-selective inhibition of HDACs. Accordingly, there is a chance that selective HDAC inhibitors may be developed as an effective therapeutic agent for various diseases (Matthias et al., Mol. Cell. Biol. 28, 1688-1701 (2008)).

HDAC6, one of class IIb HDACs, is known to be mainly present in cytoplasma and contain a tubulin protein, thus being involved in the deacetylation of a number of non-histone substrates (HSP90, cortactin, etc.) (Yao et al., Mol. Cell 18, 601-607 (2005)). HDAC6 has two catalytic domains, in which a zinc finger domain of C-terminal may bind to an ubiquitinated protein. HDAC6 is known to have a number of non-histone proteins as a substrate, and thus play an important role in various diseases such as cancer, inflammatory diseases, autoimmune diseases, neurological diseases, neurodegenerative disorders and the like (Santo et al., Blood 119, 2579-2589 (2012); Vishwakarma et al., International Immunopharmacology 16, 72-78 (2013); Hu et al., J. Neurol. Sci. 304, 1-8 (2011)).

A structural feature that various HDAC inhibitors have in common is comprised of a cap group, a linker group and a zinc-binding group (ZBG) as shown in the following structure of vorinostat. Many researchers have conducted a study on the inhibitory activity and selectivity with regard to enzymes through a structural modification of the cap group and the linker group. Out of the groups, it is known that the zinc-binding group plays a more important role in the enzyme inhibitory activity and selectivity (Wiest et al., J. Org. Chem 78, 5051-5055 (2013); Methot et al., Bioorg. Med. Chem. Lett. 18, 973-978 (2008)).

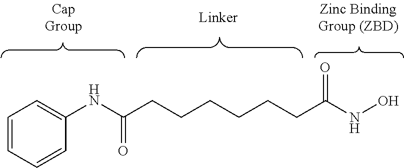 [SMILES: O=C(CCCCCCC(=O)Nc1ccccc1)NO]

Most of the zinc-binding group is hydroxamic acid or benzamide. Herein, hydroxamic acid derivatives show a strong HDAC inhibitory effect, but have a problem with low bioavailability and serious off-target activity. Benzamide derivatives include aniline, and thus have a problem in that it may produce toxic metabolites in vivo (Woster et al., Med. Chem. Commun., online publication (2015)).

Accordingly, unlike the non-selective inhibitors having side effects, there is a need to develop selective HDAC6 inhibitors, which has a zinc-binding group with improved bioavailability, while causing no side effects in order to treat cancer, inflammatory diseases, autoimmune diseases, neurological diseases, neurodegenerative disorders and the like.

### RELATED ART REFERENCES

### Patent Documents

International Unexamined Patent Publication No. WO 2011/091213 (publicized on Jul. 28, 2011): ACY-1215
International Unexamined Patent Publication No. WO 2011/011186 (publicized on Jan. 27, 2011): Tubastatin
International Unexamined Patent Publication No. WO 2013/052110 (publicized on Apr. 11, 2013): Sloan-K
International Unexamined Patent Publication No. WO 2013/041407 (publicized on Mar. 28, 2013): Cellzome
International Unexamined Patent Publication No. WO 2013/134467 (publicized on Sep. 12, 2013): Kozi
International Unexamined Patent Publication No. WO 2013/008162 (publicized on Jan. 17, 2013): Novartis
International Unexamined Patent Publication No. WO 2013/080120 (publicized on Jun. 6, 2013): Novartis
International Unexamined Patent Publication No. WO 2013/066835 (publicized on May 10, 2013): Tempero
International Unexamined Patent Publication No. WO 2013/066838 (publicized on May 10, 2013): Tempero
International Unexamined Patent Publication No. WO 2013/066833 (publicized on May 10, 2013): Tempero
International Unexamined Patent Publication No. WO 2013/066839 (publicized on May 10, 2013): Tempero

### DISCLOSURE OF THE INVENTION

### Technical Problem

An object of the present invention is to provide 1,3,4-oxadiazole thiocarbonyl compounds having a selective HDAC6 inhibitory activity, stereoisomers thereof or pharmaceutically acceptable salts thereof.

Another object of the present invention is to provide a pharmaceutical composition including 1,3,4-oxadiazole thiocarbonyl compounds having a selective HDAC6 inhibitory activity, stereoisomers thereof or pharmaceutically acceptable salts thereof.

Still another object of the present invention is to provide a method for preparing the same.

Still another object of the present invention is to provide a pharmaceutical composition containing the compounds.

Still another object of the present invention is to provide a pharmaceutical composition containing the compounds for preventing or treating HDAC6 activity-related diseases. Herein, the HDAC6 activity-related diseases may include infectious diseases, neoplasm, endocrinopathy, nutritional and metabolic diseases, mental and behavioral disorders, neurological diseases, eye and ocular adnexal diseases, circulatory diseases, respiratory diseases, digestive troubles, skin and subcutaneous tissue diseases, musculoskeletal system and connective tissue diseases or teratosis, deformities and chromosomal aberration.

Still another object of the present invention is to provide a use thereof for preparing a medicament for preventing or treating H DAC6 activity-related diseases.

Still another object of the present invention is to provide a method for treating HDAC6 activity-related diseases, including administering a therapeutically effective amount of the compounds or a pharmaceutical composition containing the compounds.

### Technical Solution to Problem

The present inventors have found an oxadiazole compound having a histone deacetylase 6 (HDAC6) inhibitory activity and have used the same in inhibiting or treating HDAC6 activity-related diseases, thereby completing the present invention.

Hereinafter, the present invention will be described in more detail. All the combinations of various elements disclosed in the present invention fall within the scope of the present invention. In addition, it cannot be seen that the scope of the present invention is limited to the specific description below.

### 1,3,4-oxadiazole Thiocarbonyl Compounds

According to the objects, the compounds provided in the present invention may be as shown in (1) to (3) below.

(1) A 1,3,4-oxadiazole thiocarbonyl compound represented by formula I below, stereoisomers thereof or pharmaceutically acceptable salts thereof:

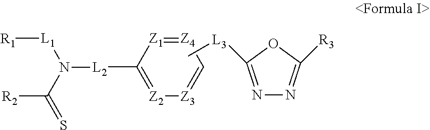

in formula I,
L₁, L₂ and L₃ are each independently a single bond or —(C₁-C₄ alkylene)-;
R₁ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl,

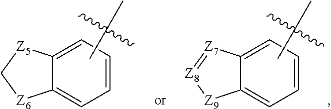

in R₁,
at least one H of —(C₁-C₄ alkyl) may be substituted with -T or —OH,
at least one H of -aryl or -heteroaryl may be each independently substituted with -T, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), -aryl, -heteroaryl,

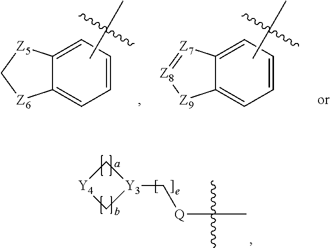

in which at least one H of

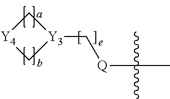

may be substituted with -T, —(C₁-C₄ alkyl), —CF₃ or —CF₂H,
at least one H of —(C₃-C₇ cycloalkyl), —(C₂-C₆ cycloheteroalkyl), -adamantyl,

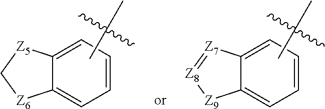

may be each independently substituted with -T, —OH or —(C₁-C₄ alkyl);
R₂ is —NR^{A}R^{B}, —OR^{C}, -heteroaryl,

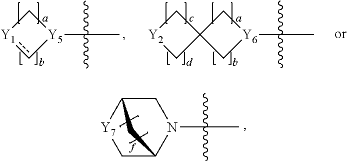

in R₂,
at least one H of

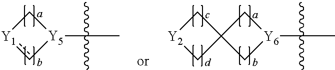

may be substituted with -T, —OH, —O(C₁-C₄ alkyl), —NR^{D}R^{E}, —(C₁-C₄ alkyl), —CF₃, —CF₂H, —CN, -aryl, -heteroaryl, —(C₁-C₄ alkyl)-aryl or —(C₁-C₄ alkyl)-heteroaryl, in which at least one H of -aryl, -heteroaryl, —(C₁-C₄ alkyl)-aryl or —(C₁-C₄ alkyl)-heteroaryl may be substituted with -T, —OH, —CF₃ or —CF₂H;
R₃ is -CT₃ or -CT₂H;
Y₁, Y₂, Y₄ and Y₇ are each independently ═CH—, —CHR^{F}—, —NR^{F}—, —O—, —C(═O)— or —S(═O)₂—;
Y₃, Y₅ and Y₆ are each independently —CH— or —N—;
Z₁ to Z₄ are each independently N or CR^{Z},
in Z₁ to Z₄,
at least three of Z₁ to Z₄ may not be N at the same time, and R^{Z} is —H, -T or —O(C₁-C₄ alkyl);
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ alkyl)-heteroaryl, —(C₃-C₇ cycloalkyl), —(C₂-C₆ heterocycloalkyl) or

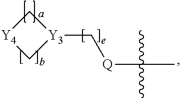

in R^{A} and R^{B},
at least one H of —(C₁-C₄ alkyl), —(C₁-C₄ alkyl)-OH or —(C₁-C₄ alkyl)-NR^{D}R^{E} may be substituted with -T,
at least one H of -aryl, —(C₁-C₄ alkyl)-aryl, -heteroaryl, —(C₁-C₄ alkyl)-heteroaryl, —(C₃-C₇ cycloalkyl) or —(C₂-C₆ heterocycloalkyl) may be substituted with -T, —OH, —O(C₁-C₄ alkyl), —(C₁-C₄ alkyl), —CF₃, —CF₂H or —CN,
at least one H of

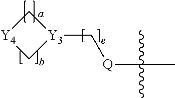

may be substituted with -T, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl or -heteroaryl;
R^{C} is —(C₁-C₄ alkyl), -aryl, —(C₁-C₄ alkyl)-aryl, -heteroaryl or —(C₁-C₄ alkyl)-heteroaryl,
in R^{C},
at least one H of —(C₁-C₄ alkyl) may be substituted with -T or —OH,
at least one H of -aryl, —(C₁-C₄ alkyl)-aryl, -heteroaryl or —(C₁-C₄ alkyl)-heteroaryl may be substituted with -T, —OH, —CF₃ or —CF₂H;
R^{D} and R^{E} are each independently —H, —(C₁-C₄ alkyl), -aryl or —(C₁-C₄ alkyl)-aryl,
in R^{D} and R^{E},
at least one H of —(C₁-C₄ alkyl) may be substituted with -T or —OH,
at least one H of -aryl or —(C₁-C₄ alkyl)-aryl may be substituted with -T, —OH, —CF₃ or —CF₂H;
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —NR^{D}R^{E}, —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl),
in R^{F},
at least one H of —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —NR^{D}R^{E}, —(C₁-C₄ alkyl)-NR^{D}R^{E} or —S(═O)₂—(C₁-C₄ alkyl) may be substituted with -T,
at least one H of -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl) may be substituted with -T, —OH, —(C₁-C₄ alkyl), —CF₃ or —CF₂H;
R^{G} is —H or —(C₁-C₄ alkyl);
Q is —O— or a single bond;
[symbol] is a single bond or a double bond, provided that when [symbol] is a double bond, Y₁ is ═CH—;
a to e are each independently an integer of 0, 1, 2, 3 or 4, provided that a and b may not be 0 together, and c and d may not be 0 together;
f is an integer of 1 or 2; and
T is F, Cl, Br or I.

(2) The 1,3,4-oxadiazole thiocarbonyl compound, stereoisomers thereof or pharmaceutically acceptable salts thereof according to above (1):
in formula I,
L₁, L₂ and L₃ are each independently a single bond or —(C₁-C₂ alkylene)-;
R₁ is —(C₁-C₄ alkyl), —(C₆-C₁₂ aryl) or —(C₃-C₁₀ heteroaryl) including at least one heteroatom selected from the group consisting of O, N and S,
in R₁,
at least one H of —(C₁-C₄ alkyl) may be substituted with -T or —OH,
at least one H of —(C₆-C₁₂ aryl) or —(C₃-C₁₀ heteroaryl) including at least one heteroatom selected from the group consisting of O, N and S may be each independently substituted with -T, —CF₃ or —CF₂H;
R₂ is —(C₃-C₁₀ heteroaryl) including at least one heteroatom selected from the group consisting of O, N and S,

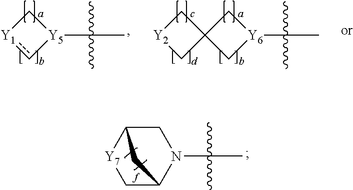

R₃ is -CT₃ or -CT₂H;
Y₁, Y₂, Y₄ and Y₇ are each independently ═CH—, —CHR^{F}—, —NR^{F}—, —O—, —C(═O)— or —S(═O)₂—;
Y₃, Y₅ and Y₆ are each independently —CH— or —N—;
Z₁ to Z₄ are each independently N or CR^{Z},
in Z₁ to Z₄,
at least three of Z₁ to Z₄ may not be N at the same time,
R^{z} is —H, -T or —O(C₁-C₄ alkyl);
R^{F} is —H, —(C₁-C₆ alkyl), —C(═O)—(C₁-C₄ alkyl) or —(C₂-C₆ heterocycloalkyl);
[symbol] is a single bond or a double bond, provided that when [symbol] is a double bond, Y₁ is ═CH—;
a to e are each independently an integer of 0, 1, 2, 3 or 4, provided that a and b may not be 0 together, and c and d may not be 0 together;
f is an integer of 1 or 2; and
T is F, Cl, Br or I.

In the present invention,

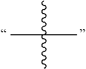

represents a linked part of the formula.

In the present invention, [symbol] represents a single bond or a double bond. In other words, [symbol] may be [symbol] as a single bond or [symbol] as a double bond.

In the present invention, the “single bond” refers to a bond in which two atoms share a pair of electrons with a bond formed.

In the present invention, “Cₘ-Cₙ” (in which m and n are each independently an integer of 1 or more) may mean the number of carbons, for example, “C₁-C₄ alkyl” represents an alkyl having 1 to 4 carbon atoms.

In the present invention, “alkyl” means a linear or branched saturated hydrocarbon group and, for example, “C₁-C₄ alkyl” may include methyl, ethyl, n-propyl, isopropyl, n-butyl, sec-butyl, tert-butyl, isobutyl, etc.

In the present invention, “alkylene” means a divalent functional group derived from the defined alkyl (including both linear and branched) and, for example, “C₁-C₄ alkylene” may include methylene (—CH₂—), ethylene (—CH₂CH₂—), n-propylene (—CH₂CH₂CH₂—), n-butylene (—CH₂CH₂CH₂CH₂—), etc.

In the present invention, “heteroaryl” means an aromatic functional group having at least one heteroatom in a ring, and the heteroatom may include at least one selected from the group consisting of O, N and S. The heteroaryl may include one which has 3 to carbon atoms in the ring. The heteroaryl may be a 4- or more membered ring, for example, a 5- to 6-membered ring. For example, “heteroaryl” may be furan, thiophene, thiazole, thiadiazole, pyrrole, pyrazole, pyridine, pyrimidine, imidazole, triazole, triazine, pyridazine, pyrazine or the like, but is not limited thereto.

In the present invention, “heterocycloalkyl” means a cyclic alkyl having at least one heteroatom in the ring. The heteroatom may include at least one selected from the group consisting of O, N and S. The heterocycloalkyl may include one which has 3 to 10 carbon atoms in the ring. The heterocycloalkyl may be a 3- or more membered ring, for example, a 3- to 6-membered ring. For example, the “heterocycloalkyl” may be propylene oxide, oxetane, tetrahydrofuran, tetrahydropyran, azetidine, morpholine, thiomorpholine dioxide, piperazine, piperidine, oxadiazole, pyrrolidine, etc., but is not limited thereto.

In the present invention, T means a halogen atom and may be F, Cl, Br or I.

In the present invention, pharmaceutically acceptable salts may refer to the salts conventionally used in a pharmaceutical industry, for example, inorganic ion salts prepared from calcium, potassium, sodium, magnesium and the like; inorganic acid salts prepared from hydrochloric acid, nitric acid, phosphoric acid, bromic acid, iodic acid, perchloric acid, sulfuric acid, etc.; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc.; sulfonic acid salts prepared from methanesulfonic acid, ethanesulfonic acid, benzenesulfonic acid, p-toluenesulfonic acid, naphthalenesulfonic acid, etc.; amino acid salts prepared from glycine, arginine, lysine, etc.; amine salts prepared from trimethylamine, triethylamine, ammonia, pyridine, picoline, etc.; and the like, but types of salts meant in the present invention are not limited to those listed salts.

“Stereoisomer” of the 1,3,4-oxadiazole thiocarbonyl compound represented by formula I of the present invention may include a diastereomer and an optical isomer (enantiomer), in which the optical isomer may include not only an enantiomer but also both a mixture of the enantiomer and even a racemate. The isomer may be separated by being split according to the related art, for example, column chromatography, HPLC or the like. Alternatively, each stereoisomer of the 1,3,4-oxadiazole thiocarbonyl compound represented by formula I may be stereospecifically synthesized by using a known array of optically pure starting materials and/or reagents.

(3) The 1,3,4-oxadiazole thiocarbonyl compound, stereoisomers thereof or pharmaceutically acceptable salts thereof according to above (1) or (2),
in which the compound is one selected from the group consisting of compounds 1 to 46 shown in table 1.

TABLE 1

| Compound | Structure |
|---|---|
| 1 | 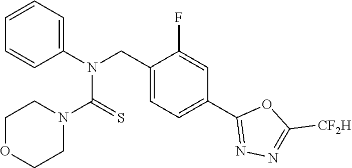 [SMILES: Fc1cc(-c2nnc(C(F)F)o2)ccc1CN(C(=S)N1CCOCC1)c1ccccc1] |
| 2 | 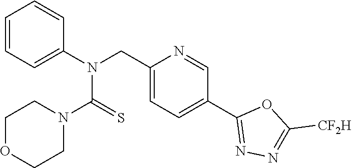 [SMILES: FC(F)c1nnc(-c2ccc(CN(C(=S)N3CCOCC3)c3ccccc3)nc2)o1] |
| 3 | 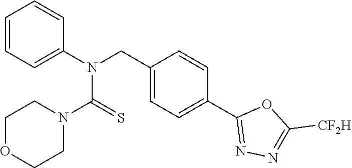 [SMILES: FC(F)c1nnc(-c2ccc(CN(C(=S)N3CCOCC3)c3ccccc3)cc2)o1] |
| 4 | 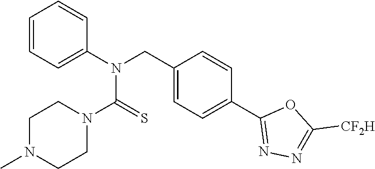 [SMILES: CN1CCN(C(=S)N(Cc2ccc(-c3nnc(C(F)F)o3)cc2)c2ccccc2)CC1] |
| 5 | 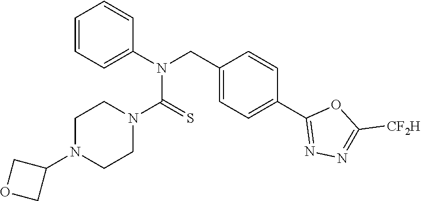 [SMILES: FC(F)c1nnc(-c2ccc(CN(C(=S)N3CCN(C4COC4)CC3)c3ccccc3)cc2)o1] |
| 6 | 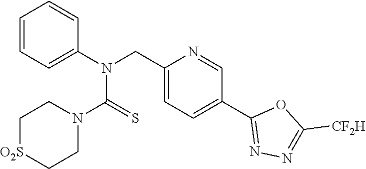 [SMILES: O=S1(=O)CCN(C(=S)N(Cc2ccc(-c3nnc(C(F)F)o3)cn2)c2ccccc2)CC1] |
| 7 | 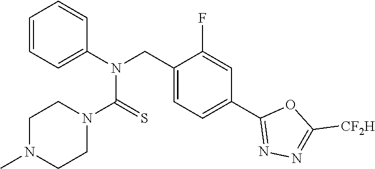 [SMILES: CN1CCN(C(=S)N(Cc2ccc(-c3nnc(C(F)F)o3)cc2F)c2ccccc2)CC1] |
| 8 | 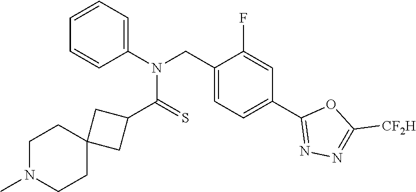 [SMILES: CN1CCC2(CC1)CC(C(=S)N(Cc1ccc(-c3nnc(C(F)F)o3)cc1F)c1ccccc1)C2] |
| 9 | 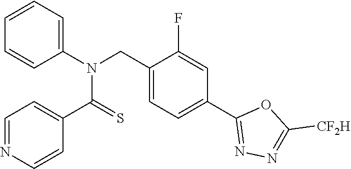 [SMILES: Fc1cc(-c2nnc(C(F)F)o2)ccc1CN(C(=S)c1ccncc1)c1ccccc1] |
| 10 | 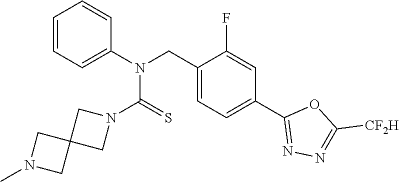 [SMILES: CN1CC2(C1)CN(C(=S)N(Cc1ccc(-c3nnc(C(F)F)o3)cc1F)c1ccccc1)C2] |
| 11 | 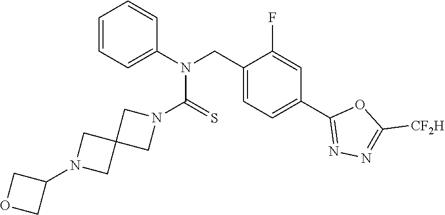 [SMILES: Fc1cc(-c2nnc(C(F)F)o2)ccc1CN(C(=S)N1CC2(C1)CN(C1COC1)C2)c1ccccc1] |
| 12 | 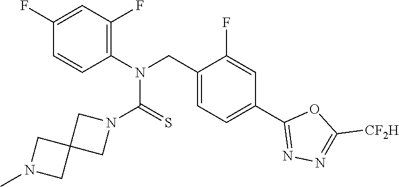 [SMILES: CN1CC2(C1)CN(C(=S)N(Cc1ccc(-c3nnc(C(F)F)o3)cc1F)c1ccc(F)cc1F)C2] |
| 13 | 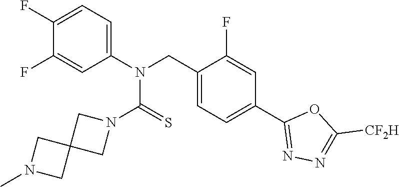 [SMILES: CN1CC2(C1)CN(C(=S)N(Cc1ccc(-c3nnc(C(F)F)o3)cc1F)c1ccc(F)c(F)c1)C2] |
| 14 | 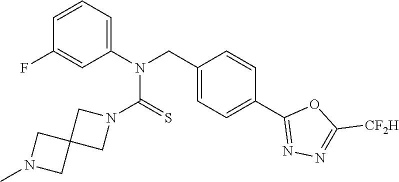 [SMILES: CN1CC2(C1)CN(C(=S)N(Cc1ccc(-c3nnc(C(F)F)o3)cc1)c1cccc(F)c1)C2] |
| 15 | 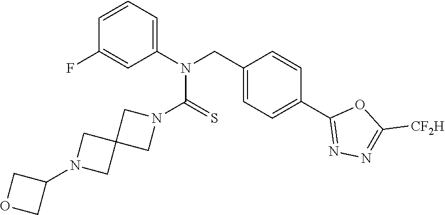 [SMILES: Fc1cccc(N(Cc2ccc(-c3nnc(C(F)F)o3)cc2)C(=S)N2CC3(C2)CN(C2COC2)C3)c1] |
| 16 | 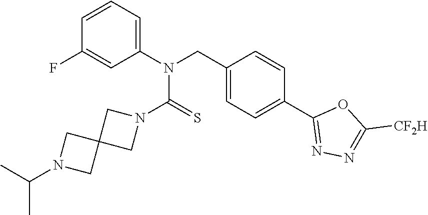 [SMILES: CC(C)N1CC2(CN(C(=S)N(Cc3ccc(-c4nnc(C(F)F)o4)cc3)c3cccc(F)c3)C2)C1] |
| 17 | 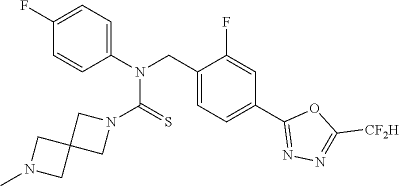 [SMILES: CN1CC2(C1)CN(C(=S)N(Cc1ccc(-c3nnc(C(F)F)o3)cc1F)c1ccc(F)cc1)C2] |
| 18 | 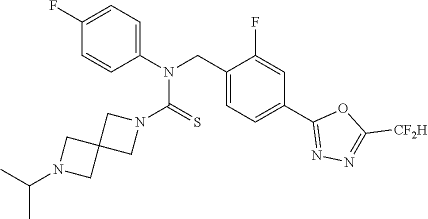 [SMILES: CC(C)N1CC2(CN(C(=S)N(Cc3ccc(-c4nnc(C(F)F)o4)cc3F)c3ccc(F)cc3)C2)C1] |
| 19 | 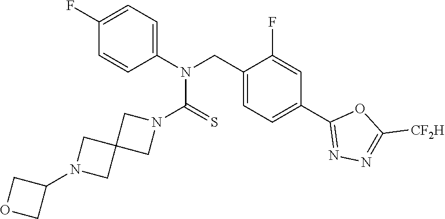 [SMILES: Fc1ccc(N(Cc2ccc(-c3nnc(C(F)F)o3)cc2F)C(=S)N2CC3(C2)CN(C2COC2)C3)cc1] |
| 20 | 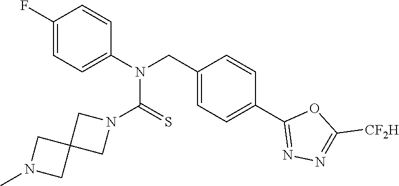 [SMILES: CN1CC2(C1)CN(C(=S)N(Cc1ccc(-c3nnc(C(F)F)o3)cc1)c1ccc(F)cc1)C2] |
| 21 | 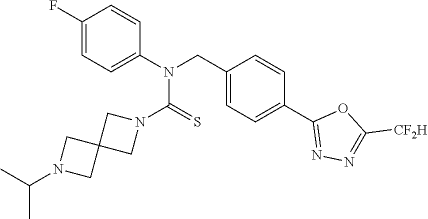 [SMILES: CC(C)N1CC2(CN(C(=S)N(Cc3ccc(-c4nnc(C(F)F)o4)cc3)c3ccc(F)cc3)C2)C1] |
| 22 | 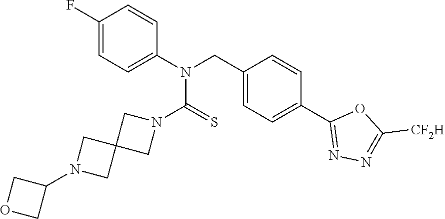 [SMILES: Fc1ccc(N(Cc2ccc(-c3nnc(C(F)F)o3)cc2)C(=S)N2CC3(C2)CN(C2COC2)C3)cc1] |
| 23 | 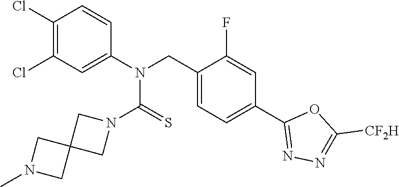 [SMILES: CN1CC2(C1)CN(C(=S)N(Cc1ccc(-c3nnc(C(F)F)o3)cc1F)c1ccc(Cl)c(Cl)c1)C2] |
| 24 | 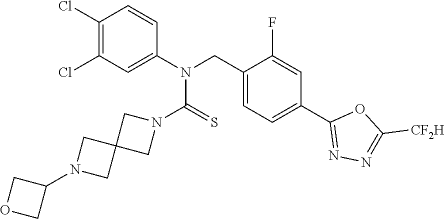 [SMILES: Fc1cc(-c2nnc(C(F)F)o2)ccc1CN(C(=S)N1CC2(C1)CN(C1COC1)C2)c1ccc(Cl)c(Cl)c1] |
| 25 | 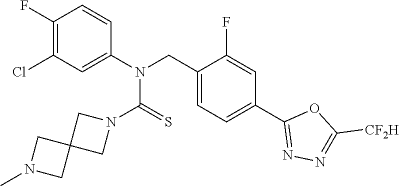 [SMILES: CN1CC2(C1)CN(C(=S)N(Cc1ccc(-c3nnc(C(F)F)o3)cc1F)c1ccc(F)c(Cl)c1)C2] |
| 26 | 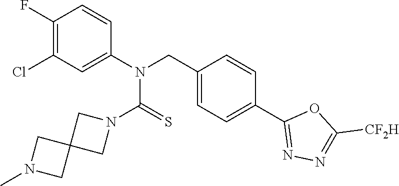 [SMILES: CN1CC2(C1)CN(C(=S)N(Cc1ccc(-c3nnc(C(F)F)o3)cc1)c1ccc(F)c(Cl)c1)C2] |
| 27 | 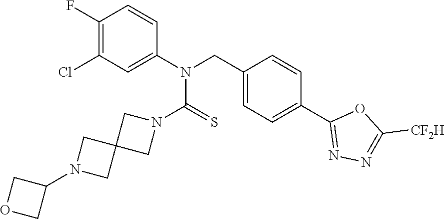 [SMILES: Fc1ccc(N(Cc2ccc(-c3nnc(C(F)F)o3)cc2)C(=S)N2CC3(C2)CN(C2COC2)C3)cc1Cl] |
| 28 | 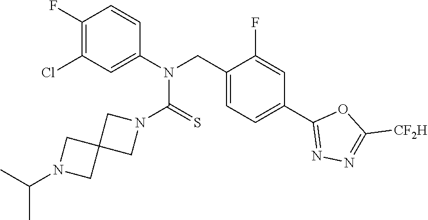 [SMILES: CC(C)N1CC2(CN(C(=S)N(Cc3ccc(-c4nnc(C(F)F)o4)cc3F)c3ccc(F)c(Cl)c3)C2)C1] |
| 29 | 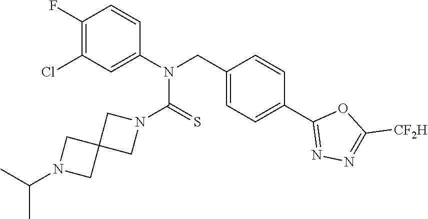 [SMILES: CC(C)N1CC2(CN(C(=S)N(Cc3ccc(-c4nnc(C(F)F)o4)cc3)c3ccc(F)c(Cl)c3)C2)C1] |
| 30 | 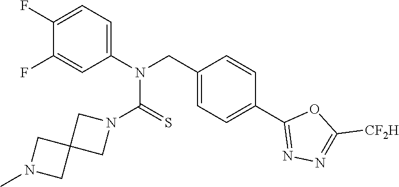 [SMILES: CN1CC2(C1)CN(C(=S)N(Cc1ccc(-c3nnc(C(F)F)o3)cc1)c1ccc(F)c(F)c1)C2] |
| 31 | 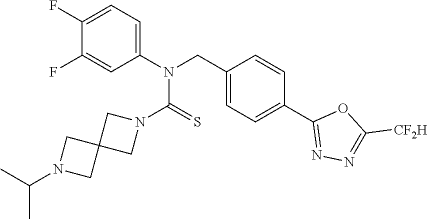 [SMILES: CC(C)N1CC2(CN(C(=S)N(Cc3ccc(-c4nnc(C(F)F)o4)cc3)c3ccc(F)c(F)c3)C2)C1] |
| 32 | 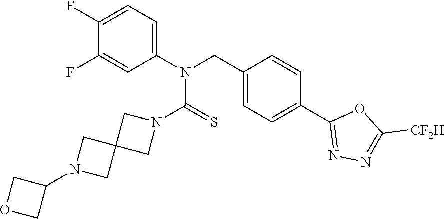 [SMILES: Fc1ccc(N(Cc2ccc(-c3nnc(C(F)F)o3)cc2)C(=S)N2CC3(C2)CN(C2COC2)C3)cc1F] |
| 33 | 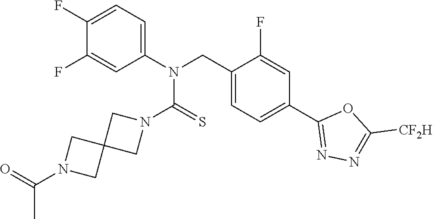 [SMILES: CC(=O)N1CC2(C1)CN(C(=S)N(Cc1ccc(-c3nnc(C(F)F)o3)cc1F)c1ccc(F)c(F)c1)C2] |
| 34 | 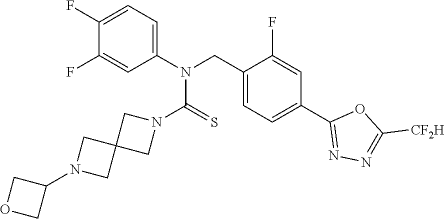 [SMILES: Fc1ccc(N(Cc2ccc(-c3nnc(C(F)F)o3)cc2F)C(=S)N2CC3(C2)CN(C2COC2)C3)cc1F] |
| 35 | 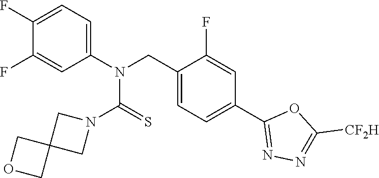 [SMILES: Fc1ccc(N(Cc2ccc(-c3nnc(C(F)F)o3)cc2F)C(=S)N2CC3(COC3)C2)cc1F] |
| 36 | 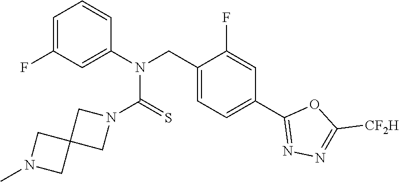 [SMILES: CN1CC2(C1)CN(C(=S)N(Cc1ccc(-c3nnc(C(F)F)o3)cc1F)c1cccc(F)c1)C2] |
| 37 | 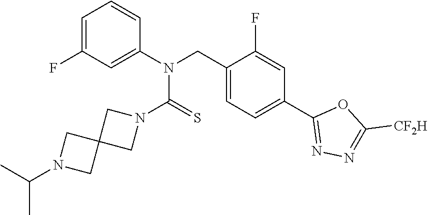 [SMILES: CC(C)N1CC2(CN(C(=S)N(Cc3ccc(-c4nnc(C(F)F)o4)cc3F)c3cccc(F)c3)C2)C1] |
| 38 | 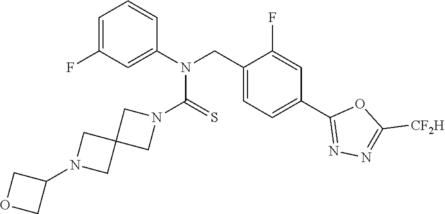 [SMILES: Fc1cccc(N(Cc2ccc(-c3nnc(C(F)F)o3)cc2F)C(=S)N2CC3(C2)CN(C2COC2)C3)c1] |
| 39 | 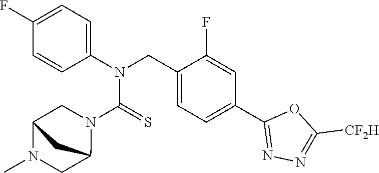 [SMILES: CN1C[C@@H]2C[C@H]1CN2C(=S)N(Cc1ccc(-c2nnc(C(F)F)o2)cc1F)c1ccc(F)cc1] |
| 40 | 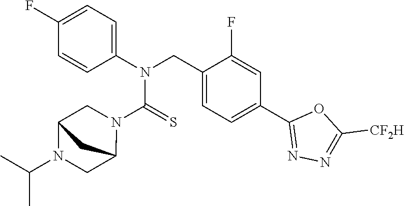 [SMILES: CC(C)N1C[C@@H]2C[C@H]1CN2C(=S)N(Cc1ccc(-c2nnc(C(F)F)o2)cc1F)c1ccc(F)cc1] |
| 41 | 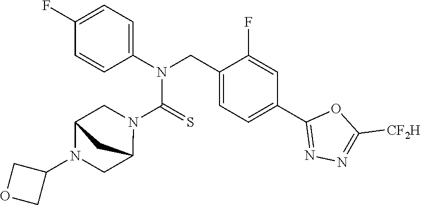 [SMILES: Fc1ccc(N(Cc2ccc(-c3nnc(C(F)F)o3)cc2F)C(=S)N2C[C@@H]3C[C@H]2CN3C2COC2)cc1] |
| 42 | 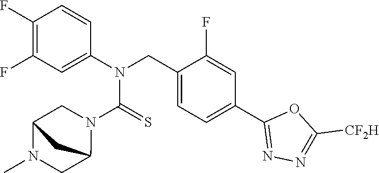 [SMILES: CN1C[C@@H]2C[C@H]1CN2C(=S)N(Cc1ccc(-c2nnc(C(F)F)o2)cc1F)c1ccc(F)c(F)c1] |
| 43 | 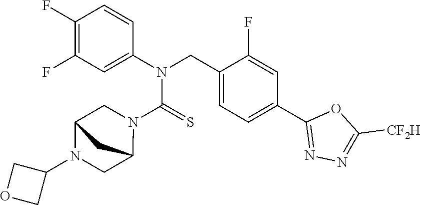 [SMILES: Fc1ccc(N(Cc2ccc(-c3nnc(C(F)F)o3)cc2F)C(=S)N2C[C@@H]3C[C@H]2CN3C2COC2)cc1F] |
| 44 | 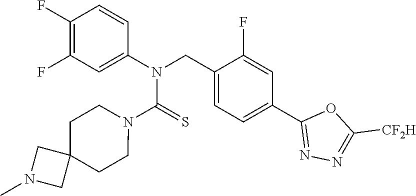 [SMILES: CN1CC2(CCN(C(=S)N(Cc3ccc(-c4nnc(C(F)F)o4)cc3F)c3ccc(F)c(F)c3)CC2)C1] |
| 45 | 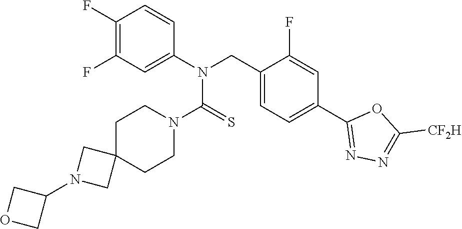 [SMILES: Fc1ccc(N(Cc2ccc(-c3nnc(C(F)F)o3)cc2F)C(=S)N2CCC3(CC2)CN(C2COC2)C3)cc1F] |
| 46 | 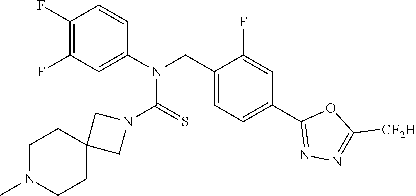 [SMILES: CN1CCC2(CC1)CN(C(=S)N(Cc1ccc(-c3nnc(C(F)F)o3)cc1F)c1ccc(F)c(F)c1)C2] |

Method for Preparing 1,3,4-Oxadiazole Thiocarbonyl Compounds of formula I

The 1,3,4-oxadiazole thiocarbonyl compound represented by formula I, stereoisomers thereof or pharmaceutically acceptable salts thereof may be prepared according to a preparation method represented by reaction formulas 1 to 4, and even a preparation method modified at a level apparent to those skilled in the art may be also included therein.

Hereinafter, in the reaction formulas, X₁ to X₄ may be sequentially the same as Z₁ to Z₄ of formula I, and other symbols may be represented by the same symbols as those of formula I in the reaction formulas, and those not specifically described may be the same as defined in formula I. Thus, any redundant description will be omitted.

In the following reaction formulas 1 to 4, the substituent represented by “X” may mean a leaving group.

In the following reaction formulas 1 to 4, “PG” may represent an amine protecting group and, for example, the PG may be a tert-butyloxycarbonyl group (BOC).

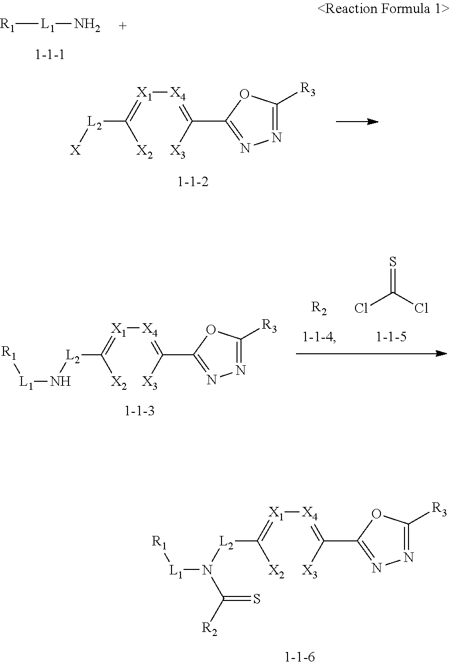

In the reaction formula 1, the compound of formula 1-1-4 represented by “R₂” may mean a compound in which a primary or secondary amine group is introduced into R₂, which is a monovalent substituent, in the definition of formula I.

According to the reaction formula 1, a compound of formula 1-1-3 may be prepared through a substitution reaction between a compound of formula 1-1-1 and a compound of formula 1-1-2, after which a compound of formula 1-1-4 and a compound of formula 1-1-5 may be reacted to prepare a compound of formula 1-1-6.

The compound prepared by the reaction formula 1 may be compounds 1, 2, 3, 7, 35, etc.

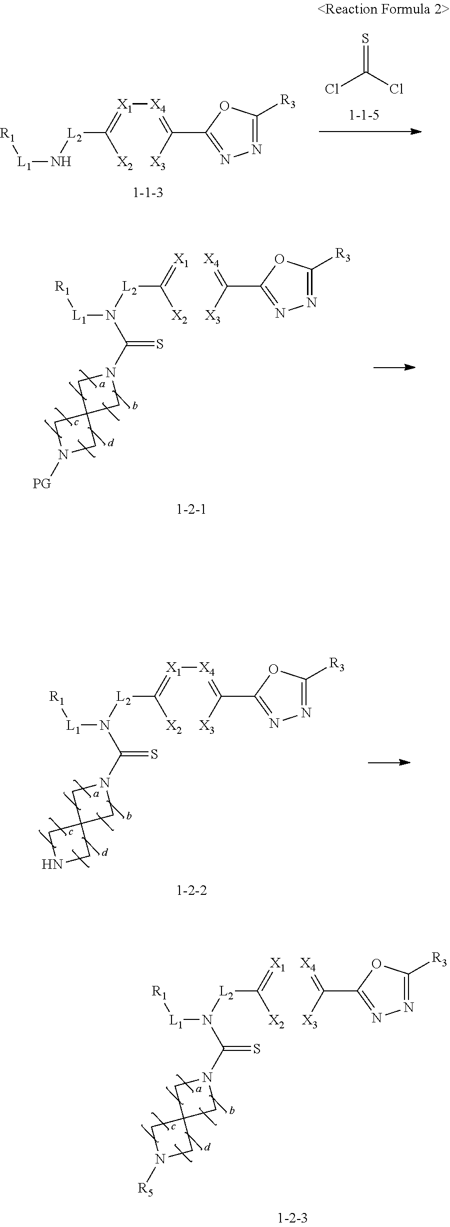

In the reaction formula 2, R₅ may be the same as defined as R^{F} in formula I.

According to the reaction formula 2, a compound of formula 1-2-1 may be prepared by reacting a compound of formula 1-1-3, a compound of formula 1-1-5, and a spiro compound into which an amine group including a protecting group (PC) is introduced. After that, the protecting group may be removed to prepare a compound of formula 1-2-2, and then a reductive amination reaction or a substitution reaction may be performed to prepare a compound of formula 1-2-3.

The compound prepared by the reaction formula 2 may be 10, 11, 12, 13, 14, 15, 16, 17, 18, 19, 20, 21, 22, 23, 24, 25, 26, 27, 28, 29, 30, 31, 32, 33, 34, 36, 37, 38, 44, 45, 46, etc.

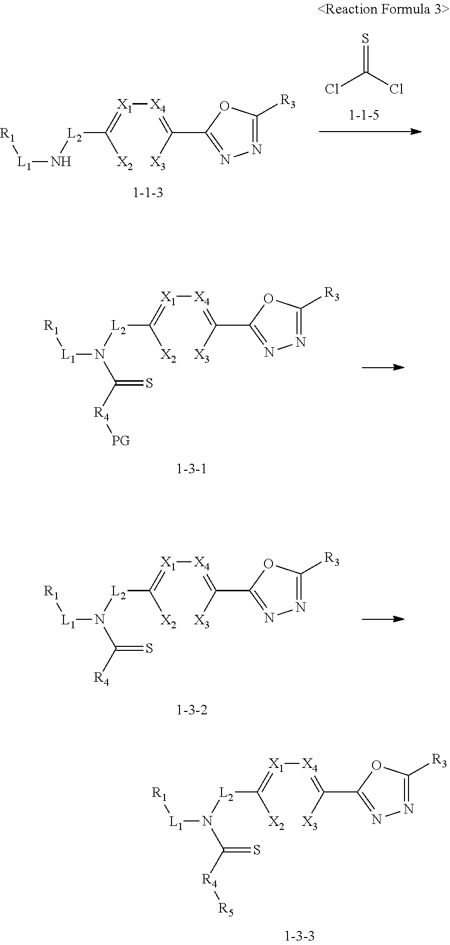

In the reaction formula 3, R₄ may be

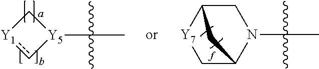

(in which Y₁ and Y₇ may each independently represent —N—), and R₅ may be the same as defined as R^{F} in formula I.

According to the reaction formula 3, a compound of formula 1-3-1 may be prepared by reacting a compound of formula 1-1-3, a compound of formula 1-1-5, and a R₄ compound into which an amine group including a protecting group (PG) is introduced. After that, the protecting group may be removed to prepare a compound of formula 1-3-2, and then a reductive amination reaction or a substitution reaction may be performed to prepare a compound of formula 1-3-3.

The compound prepared by the reaction formula 3 may be compounds 4, 5, 39, 40, 41, 42, 43, etc.

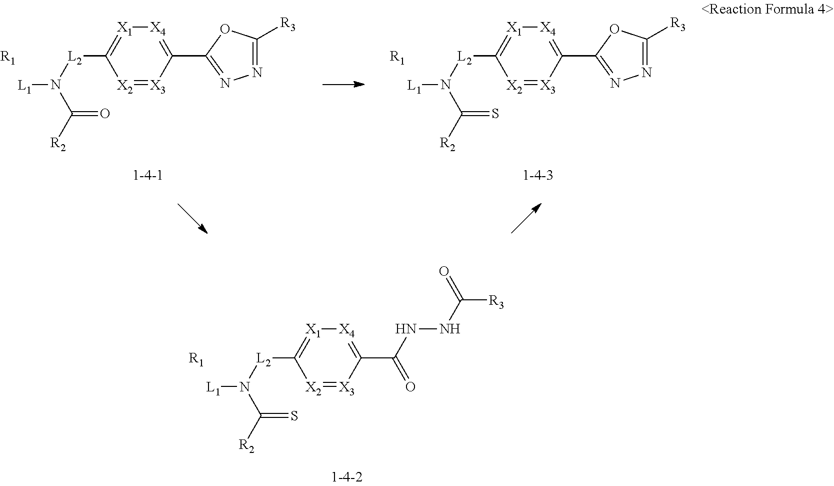

According to the reaction formula 4, a compound of formula 1-4-1 may be reacted with 2,4-bis(4-methoxyphenyl)-1,3,2,4-dithiadiphosphetan-2,4-disulfide (Lawesson's reagent) to prepare a compound of formula 1-4-2 or formula 1-4-3.

Alternatively, the compound of formula 1-4-2 may be reacted with 1-methoxy-N-triethylammoniosulfonyl-methanimidate (Burgess reagent) to prepare the compound of formula 1-4-3.

The compound prepared by the reaction formula 4 may be compounds 6, 8, 9, etc.

### Composition Including 1,3,4-Oxadiazole Thiocarbonyl Compound Represented by Formula I, Use Thereof and Therapeutic Method Using the Same

The present invention provides a pharmaceutical composition including a 1,3,4-oxadiazole thiocarbonyl compound represented by formula I, stereoisomers thereof or pharmaceutically acceptable salts thereof as an active ingredient.

In addition, the present invention provides a pharmaceutical composition for preventing or treating histone deacetylase 6 activity-related diseases, including a 1,3,4-oxadiazole thiocarbonyl compound represented by formula I, stereoisomers thereof or pharmaceutically acceptable salts thereof as an active ingredient.

The pharmaceutical composition of the present invention selectively inhibits histone deacetylase 6, thereby showing a remarkable effect on preventing or treating histone deacetylase 6 activity-related diseases.

The histone deacetylase 6 activity-related diseases may include: infectious diseases such as prion disease; neoplasm such as benign tumor (e.g., myelodysplastic syndrome) or malignant tumor (e.g., multiple myeloma, lymphoma, leukemia, lung cancer, colorectal cancer, colon cancer, prostate cancer, urothelial carcinoma, breast cancer, melanoma, skin cancer, liver cancer, brain cancer, stomach cancer, ovarian cancer, pancreatic cancer, head and neck cancer, oral cancer or glioma); endocrinopathy, nutritional and metabolic diseases such as Wilson's disease, amyloidosis or diabetes; mental and behavioral disorders such as depression, rett syndrome or the like; neurological diseases such as central nervous system atrophy (e.g., Huntington's disease, spinal muscular atrophy (SMA), spinocerebellar ataxia (SCA)), neurodegenerative disease (e.g., Alzheimer's disease), motor disorder (e.g., Parkinson's disease), neuropathy (e.g., hereditary neuropathy (Charcot-Marie-Tooth disease), sporadic neuropathy, inflammatory neuropathy, drug-induced neuropathy), motor neuropathy (e.g., amyotrophic lateral sclerosis (ALS)), central nervous system demyelinating disease (e.g., multiple sclerosis (MS)), or the like; eye and ocular adnexal diseases such as uveitis; circulatory diseases such as atrial fibrillation, stroke or the like; respiratory diseases such as asthma; digestive troubles such as alcoholic liver disease, inflammatory bowel disease, Crohn's disease, ulcerative bowel disease or the like; skin and subcutaneous tissue diseases such as psoriasis; musculoskeletal system and connective tissue diseases such as rheumatoid arthritis, osteoarthritis, systemic lupus erythematosis (SLE) or the like; or teratosis, deformities and chromosomal aberration such as autosomal dominant polycystic kidney disease, and also may include other symptoms or diseases related to abnormal functions of histone deacetylase.

For administration, the pharmaceutical composition of the present invention may further include at least one type of a pharmaceutically acceptable carrier, in addition to the 1,3,4-oxadiazole thiocarbonyl compound represented by formula I, stereoisomers thereof or pharmaceutically acceptable salts thereof. The pharmaceutically acceptable carrier used herein may include saline solution, sterilized water, Ringer's solution, buffered saline, dextrose solution, maltodextrin solution, glycerol, ethanol and a mixture of at least one component thereof, and may be also used with the addition of other conventional additives such as antioxidants, buffer solutions, bacteriostatic agents, etc., if needed. In addition, diluents, dispersing agents, surfactants, binders and lubricants may be added to be formulated into injectable dosage forms such as aqueous solutions, suspensions, emulsions, etc., pills, capsules, granules or tablets. Thus, the composition of the present invention may be patches, liquid medicines, pills, capsules, granules, tablets, suppositories, etc. Such preparations may be prepared according to a conventional method used for formulation in the art or a method disclosed in Remington's Pharmaceutical Science (latest edition), Merck Publishing Company, Easton PA, and such composition may be formulated into various preparations depending on each disease or component.

The composition of the present invention may be orally or parenterally administered (for example, applied intravenously, hypodermically, intraperitoneally or locally) according to a targeted method, in which a dosage thereof varies in a range thereof depending on a patient's weight, age, gender, health condition and diet, an administration time, an administration method, an excretion rate, a severity of a disease and the like. A daily dosage of the 1,3,4-oxadiazole thiocarbonyl compound represented by formula I of the present invention may be about 1 to about 1000 mg/kg, preferably about 5 to about 100 mg/kg, and may be administered at one time a day or several times a day by dividing the daily dosage of the compound.

In addition to the 1,3,4-oxadiazole thiocarbonyl compound represented by formula I, stereoisomers thereof or pharmaceutically acceptable salts thereof, the pharmaceutical composition of the present invention may further include at least one active ingredient which shows the same or similar medicinal effects.

The present invention may provide a method for preventing or treating histone deacetylase 6 activity-related diseases, including administering a therapeutically effective amount of the 1,3,4-oxadiazole thiocarbonyl compound represented by formula I, stereoisomers thereof or pharmaceutically acceptable salts thereof.

As used herein, the term “therapeutically effective amount” may refer to an amount of the 1,3,4-oxadiazole thiocarbonyl compound represented by formula I, which is effective in preventing or treating histone deacetylase 6 activity-related diseases.

In addition, the present invention may provide a method for selectively inhibiting HDAC6 by administering the 1,3,4-oxadiazole thiocarbonyl compound represented by formula I, stereoisomers thereof or pharmaceutically acceptable salts thereof into mammals including humans.

The method for preventing or treating histone deacetylase 6 activity-related diseases according to the present invention may include not only dealing with the diseases per se before expression of symptoms, but also inhibiting or avoiding such symptoms by administering the 1,3,4-oxadiazole thiocarbonyl compound represented by formula I. In managing the disease, a preventive or therapeutic dose of a certain active ingredient may vary depending on a nature and severity of the disease or condition and a route of administering the active ingredient. A dose and a frequency thereof may vary depending on an individual patient's age, weight and reactions. A suitable dose and usage may be easily selected by those skilled in the art, naturally considering such factors. In addition, the method for preventing or treating histone deacetylase 6 activity-related diseases of the present invention may further include administering a therapeutically effective amount of an additional active agent, which is helpful in treating the diseases, along with the 1,3,4-oxadiazole thiocarbonyl compound represented by formula I, in which the additional active agent may show a synergy effect or an adjuvant effect together with the compound of the formula I.

The present invention provides a use of the 1,3,4-oxadiazole thiocarbonyl compound represented by formula I, stereoisomers thereof or pharmaceutically acceptable salts thereof in preparing a medicament for treating histone deacetylase 6 activity-related diseases. The 1,3,4-oxadiazole thiocarbonyl compound represented by formula I for preparing a medicament may be combined with an acceptable adjuvant, diluent, carrier, etc., and may be prepared into a complex agent together with other active agents, thus having a synergy action.

Matters mentioned in the use, composition and therapeutic method of the present invention may be equally applied, if not contradictory to each other.

### Advantageous Effects of Invention

According to the present invention, the 1,3,4-oxadiazole thiocarbonyl compound represented by formula I, stereoisomers thereof, or pharmaceutically acceptable salts thereof may selectively inhibit HDAC6, thus having a remarkably excellent effect of preventing or treating histone deacetylase 6 activity-related diseases.

### MODE FOR INVENTION

Hereinafter, the present invention will be described in detail through preferred Examples for better understanding of the present invention. However, the following Examples are provided only to illustrate the present invention, and thus the present invention is not limited thereto.

### Preparation of 1,3,4-oxadiazole Thiocarbonyl Compounds

### Example 1: Synthesis of compound t, N-(4-(5-(difluoromethyl)-1,3,4-oxadiazol-2-yl)-2-fluorobenzyl)-N-phenylmorpholin-4-carbothioamide

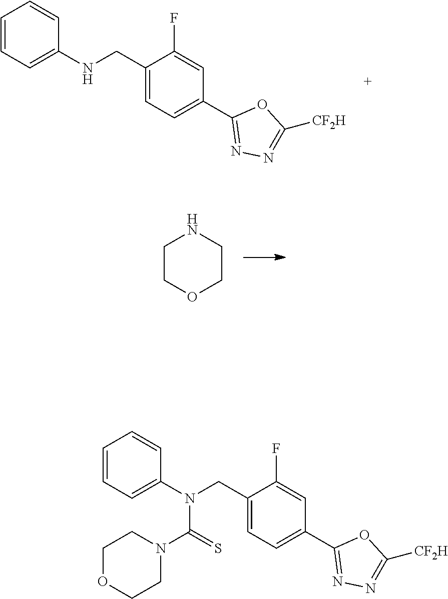 [SMILES: Fc1cc(-c2nnc(C(F)F)o2)ccc1CNc1ccccc1 ; C1COCCN1 ; Fc1cc(-c2nnc(C(F)F)o2)ccc1CN(C(=S)N1CCOCC1)c1ccccc1]

N-(4-(5-(difluoromethyl)-1,3,4-oxadiazol-2-yl)-2-fluorobenzyl)aniline (0.500 g, 1.566 mmol), N,N-diisopropylethylamine (1.091 mL, 6.264 mmol) and thiophosgene (0.268 g, 2.349 mmol) were dissolved in dichloromethane (10 mL), after which the resulting solution was stirred at 0° C. for 30 minutes and then morpholine (0.135 mL, 1.566 mmol) was added thereinto and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 30%) and concentrated to obtain a title compound (0.090 g, 12.8%) as a yellow oil form.

¹H NMR (400 MHz, CDCl₃) δ 7.86 (dd, J=8.1, 1.3 Hz, 1H), 7.80˜7.76 (m, 2H), 7.35 (t, J=7.9 Hz, 2H), 7.17-7.11 (m, 3H), 7.05 (s, 0.25H), 6.92 (s, 0.5H), 6.79 (s, 0.25H), 5.51 (s, 2H), 3.67 (t, J=4.8 Hz, 4H), 3.51 (t, J=4.8 Hz, 4H); LRMS (ES) m/z 449.4 (M⁺+1).

### Example 2: Synthesis of compound 2, N-((5-(5-(difluoromethyl)-1,3,4-oxadiazol-2-yl)pyridin-2-yl)methyl)-N-phenylmorpholin-4-carbothioamide

### [Step 1] Synthesis of N-((5-(5-(difluoromethyl)-1,3,4-oxadiazol-2-yl)pyridin-2-yl)methyl)aniline

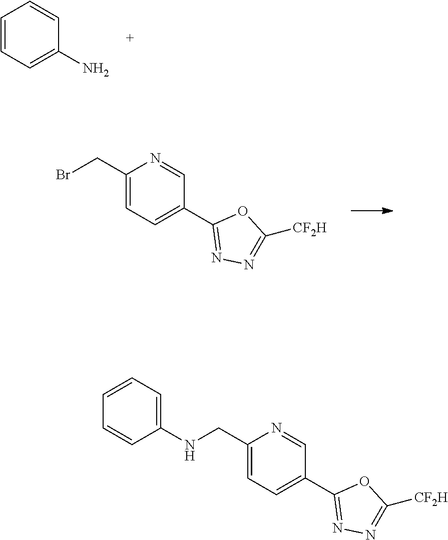 [SMILES: Nc1ccccc1 ; FC(F)c1nnc(-c2ccc(CBr)nc2)o1 ; FC(F)c1nnc(-c2ccc(CNc3ccccc3)nc2)o1]

Aniline (0.294 mL, 3.221 mmol) was dissolved in N,N-dimethylformamide (20 mL) at 0° C., after which sodium hydride (60.00%, 0.193 g, 4.832 mmol) was added into the resulting solution and stirred at the same temperature for 30 minutes. 2-(6-(bromomethyl)pyridin-3-yl)-5-(difluoromethyl)-1,3,4-oxadiazole (0.934 g, 3.221 mmol) was added into the reaction mixture and further stirred at room temperature for three hours. Solvent was removed from the reaction mixture under reduced pressure, after which water was poured into the resulting concentrate and an organic layer was extracted with ethyl acetate. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 50%) and concentrated to obtain a desired title compound (0.337 g, 34.6%) as a yellow oil form.

### [Step 2] Synthesis of Compound 2

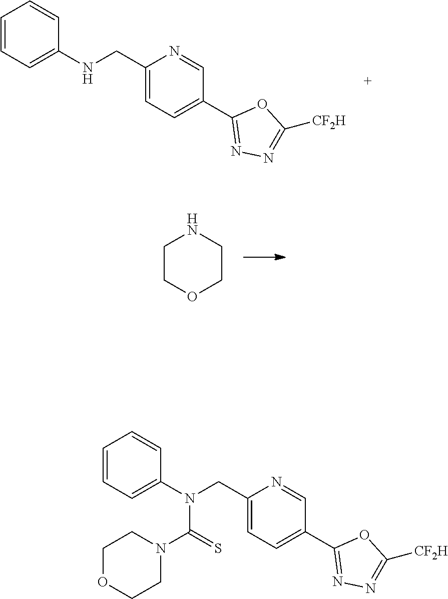 [SMILES: FC(F)c1nnc(-c2ccc(CNc3ccccc3)nc2)o1 ; C1COCCN1 ; FC(F)c1nnc(-c2ccc(CN(C(=S)N3CCOCC3)c3ccccc3)nc2)o1]

N-((5-(5-(difluoromethyl)-1,3,4-oxadiazol-2-yl)pyridin-2-yl)methyl)aniline (0.186 g, 0.615 mmol) prepared in step 1, morpholine (0.053 mL, 0.615 mmol) and N,N-diisopropylethylamine (0.429 mL, 2.461 mmol) were dissolved in dichloromethane (10 mL), after which thiophosgene (0.106 g, 0.923 mmol) was added to the resulting solution at 0° C., stirred at the same temperature for 30 minutes, and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 30%) and concentrated to obtain a desired title compound (0.030 g, 11.3%) as a colorless oil form.

¹H NMR (400 MHz, CDCl₃) δ 9.26 (d, J=2.1 Hz, 1H), 8.34 (dd, J=8.2, 2.2 Hz, 1H), 7.69 (d, J=8.2 Hz, 1H), 7.35 (t, J=7.9 Hz, 2H), 7.19-7.12 (m, 3H), 7.07 (s, 0.25H), 6.94 (s, 0.5H), 6.81 (s, 0.25H), 5.65 (s, 2H), 3.68 (t, J=4.7 Hz, 4H), 3.55 (t, J=4.8 Hz, 4H); LRMS (ES) m/z 432.4 (M⁺+1)

### Example 3: Synthesis of compound 3, N-(4-(5-(difluoromethyl)-1,3,4-oxadiazol-2-yl)benzyl)-N-phenylmorpholin-4-carbothioamide

### [Step 1] Synthesis of N-(4-(5-(difluoromethyl)-1,3,4-oxadiazol-2-yl)benzyl)aniline

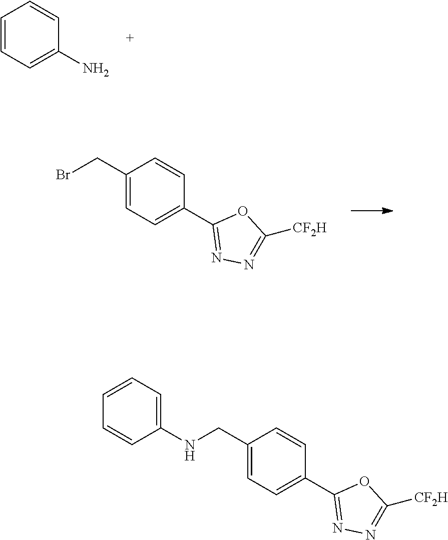 [SMILES: Nc1ccccc1 ; FC(F)c1nnc(-c2ccc(CBr)cc2)o1 ; FC(F)c1nnc(-c2ccc(CNc3ccccc3)cc2)o1]

Aniline (0.490 mL, 5.369 mmol) was dissolved in N,N-dimethylformamide (20 mL) at 0° C., after which sodium hydride (60.00%, 0.322 g, 8.053 mmol) was added into the resulting solution and stirred at the same temperature for 30 minutes. 2-(4-(bromomethyl)phenyl)-5-(difluoromethyl)-1,3,4-oxadiazole (1.552 g, 5.369 mmol) was added into the reaction mixture and further stirred at room temperature for three hours. Solvent was removed from the reaction mixture under reduced pressure, after which water was poured into the resulting concentrate and an organic layer was extracted with ethyl acetate. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 50%) and concentrated to obtain a title compound (0.550 g, 34.0%) as a white solid form.

### [Step 2] Synthesis of Compound 3

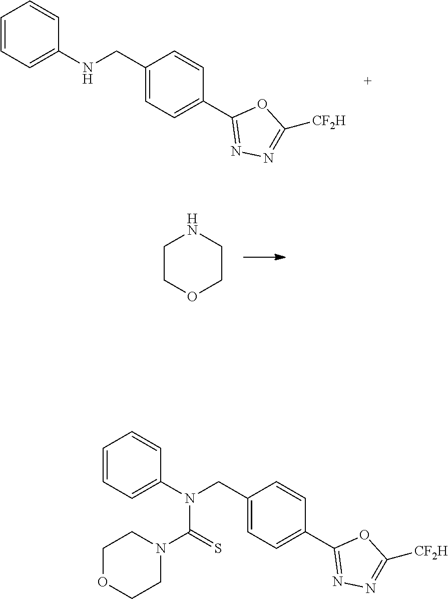 [SMILES: FC(F)c1nnc(-c2ccc(CNc3ccccc3)cc2)o1 ; C1COCCN1 ; FC(F)c1nnc(-c2ccc(CN(C(=S)N3CCOCC3)c3ccccc3)cc2)o1]

N-(4-(5-(difluoromethyl)-1,3,4-oxadiazol-2-yl)benzyl)aniline (0.300 g, 0.996 mmol) prepared in step 1 and N,N-diisopropylethylamine (0.694 mL, 3.983 mmol) were dissolved in dichloromethane (10 mL), after which morpholine (0.086 mL, 0.996 mmol) and thiophosgene (0.172 g, 1.494 mmol) were added to the resulting solution at 0° C., stirred at the same temperature for 30 minutes, and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 30%) and concentrated to obtain a title compound (0.100 g, 23.3%) as a colorless oil form.

¹H NMR (400 MHz, CDCl₃) δ 8.03 (d, J=8.3 Hz, 2H), 7.55 (d, J=8.2 Hz, 2H), 7.33˜7.28 (m, 2H), 7.12 (t, J=7.4 Hz, 1H), 7.06-7.04 (m, 2H), 7.06 (s, 0.25H), 6.91 (s, 0.5H), 6.78 (s, 0.25H), 3.65 (t, J=4.8 Hz, 4H), 3.50 (t, J=4.8 Hz, 4H); LRMS (ES) m/z 431.4 (M⁺+1)

### Example 4: Synthesis of compound 4, N-(4-(5-(difluoromethyl)-1,3,4-oxadiazol-2-yl)benzyl)-4-methyl-N-phenylpiperazin-1-carbothioamide

### [Step 1] Synthesis of tert-butyl 4-((4-(5-(difluoromethyl)-1,3,4-oxadiazol-2-yl)benzyl)(phenyl)carbamothioyl)piperazin-1-carboxylate

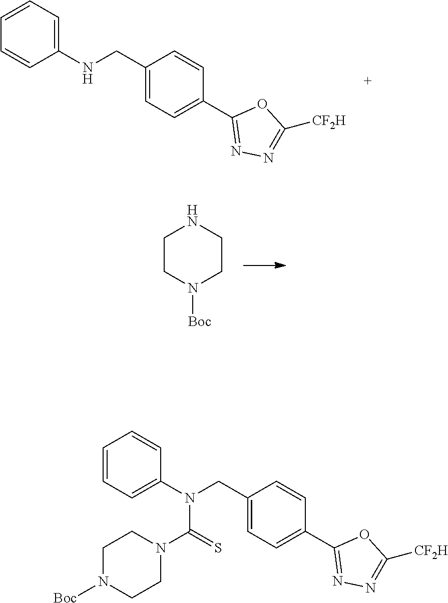 [SMILES: FC(F)c1nnc(-c2ccc(CNc3ccccc3)cc2)o1 ; CC(C)(C)OC(=O)N1CCNCC1 ; CC(C)(C)OC(=O)N1CCN(C(=S)N(Cc2ccc(-c3nnc(C(F)F)o3)cc2)c2ccccc2)CC1]

N-(4-(5-(difluoromethyl)-1,3,4-oxadiazol-2-yl)benzyl)aniline (0.677 g, 2.247 mmol) prepared by the same method as described in step 1 of compound 3, tert-butyl piperazin-1-carboxylate (0.419 g, 2.247 mmol) and N,N-diisopropylethylamine (1.565 mL, 8.988 mmol) were dissolved in dichloromethane (10 mL), after which thiophosgene (0.388 g, 3.370 mmol) was added to the resulting solution at 0° C., stirred at the same temperature for 30 minutes, and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 30%) and concentrated to obtain a title compound (0.600 g, 50.4%) as a yellow oil form.

### [Step 2] Synthesis of N-(4-(5-(difluoromethyl)-1,3,4-oxadiazol-2-yl)benzyl)-N-phenylpiperazin-1-carbothioamide

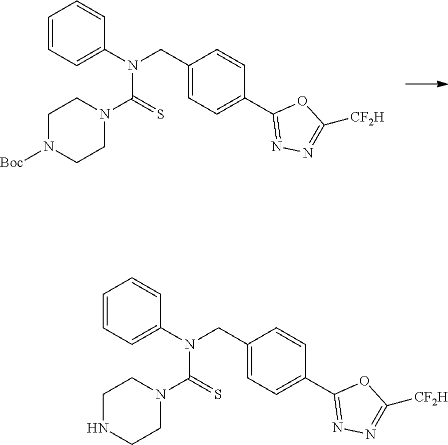 [SMILES: CC(C)(C)OC(=O)N1CCN(C(=S)N(Cc2ccc(-c3nnc(C(F)F)o3)cc2)c2ccccc2)CC1 ; FC(F)c1nnc(-c2ccc(CN(C(=S)N3CCNCC3)c3ccccc3)cc2)o1]

Tert-butyl 4-((4-(5-(difluoromethyl)-1,3,4-oxadiazol-2-yl)benzyl)(phenyl)carbamothioyl)piperazin-1-carboxylate (0.600 g, 1.133 mmol) prepared in step 1 and trifluoroacetic acid (0.868 mL, 11.329 mmol) were dissolved in dichloromethane (20 mL) at room temperature, after which the resulting solution was stirred at the same temperature for five hours. Solvent was removed from the reaction mixture under reduced pressure, after which saturated aqueous sodium hydrogen carbonate solution was poured into the resulting concentrate and an organic layer was extracted with dichloromethane. The organic layer was washed with saturated aqueous sodium chloride solution, dehydrated with anhydrous sodium sulfate, filtered, and concentrated under reduced pressure. An obtained product was used without a further purification process (0.450 g, 92.5%, white solid).

### [Step 3] Synthesis of Compound 4

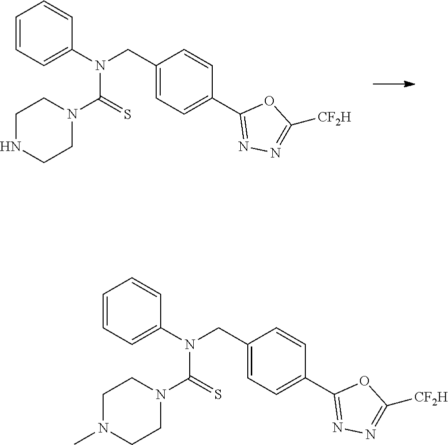 [SMILES: FC(F)c1nnc(-c2ccc(CN(C(=S)N3CCNCC3)c3ccccc3)cc2)o1 ; CN1CCN(C(=S)N(Cc2ccc(-c3nnc(C(F)F)o3)cc2)c2ccccc2)CC1]

N-(4-(5-(difluoromethyl)-1,3,4-oxadiazol-2-yl)benzyl)-N-phenylpiperazin-1-carbothioamide (0.200 g, 0.466 mmol) prepared in step 2, formaldehyde (0.028 g, 0.931 mmol) and sodium triacetoxyborohydride (0.197 g, 0.931 mmol) were dissolved in dichloromethane (10 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.050 g, 24.2%) as a colorless oil form.

¹H NMR (400 MHz, CDCl₃) δ 8.05 (d, J=8.3 Hz, 2H), 7.56 (d, J=8.3 Hz, 2H), 7.32˜7.28 (m, 2H), 7.12 (t, J=7.4 Hz, 1H), 7.04 (d, J=7.9 Hz, 2H), 7.04 (s, 0.25H), 6.91 (s, 0.5H), 6.78 (s, 0.25H), 5.52 (s, 2H), 3.69 (t, J=4.9 Hz, 4H), 2.28 (t, J=5.0 Hz, 4H), 2.23 (s, 3H); LRMS (ES) m/z 444.3 (M⁺+1).

### Example 5: Synthesis of compound 5, N-(4-(5-(difluoromethyl)-1,3,4-oxadiazol-2-yl)benzyl)-4-(oxetan-3-yl)-N-phenylpiperazin-1-carbothioamide

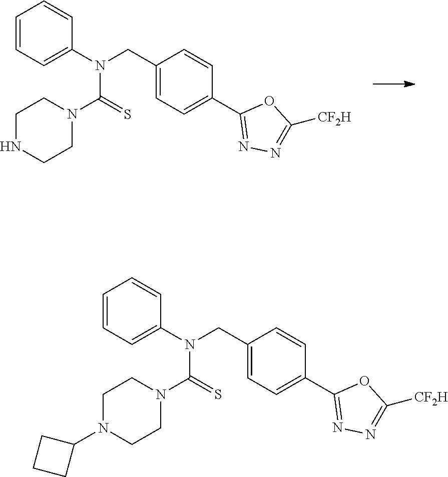 [SMILES: FC(F)c1nnc(-c2ccc(CN(C(=S)N3CCNCC3)c3ccccc3)cc2)o1 ; FC(F)c1nnc(-c2ccc(CN(C(=S)N3CCN(C4CCC4)CC3)c3ccccc3)cc2)o1]

N-(4-(5-(difluoromethyl)-1,3,4-oxadiazol-2-yl)benzyl)-N-phenylpiperazin-1-carbothioamide (0.200 g, 0.466 mmol) prepared by the same method as described in step 2 of compound 4, 3-oxetanone (0.055 mL, 0.931 mmol) and sodium triacetoxyborohydride (0.197 g, 0.931 mmol) were dissolved in dichloromethane (10 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.100 g, 44.2%) as a colorless oil form.

¹H NMR (400 MHz, CDCl₃) δ 8.04 (d, J=8.3 Hz, 2H), 7.55 (d, J=8.2 Hz, 2H), 7.32˜7.28 (m, 2H), 7.14˜7.10 (m, 1H), 7.04˜7.02 (m, 2H), 7.04 (s, 0.25H), 6.91 (s, 0.5H), 6.78 (s, 0.25H), 5.51 (s, 2H), 4.62 (t, J=6.6 Hz, 2H), 4.52 (t, J=6.1 Hz, 2H), 3.70 (t, J=4.9 Hz, 4H), 3.44˜3.38 (m, 1H), 2.19 (t, J=5.0 Hz, 4H); LRMS (ES) m/z 486.4 (M⁺+1).

### Example 6: Synthesis of compound 6, N-((5-(5-(difluoromethyl)-1,3,4-oxadiazol-2-yl)pyridin-2-yl)methyl)-N-phenylthiomorpholin-4-carbothioamide 1,1-dioxide

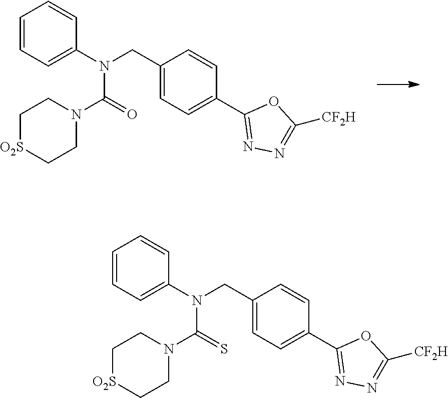 [SMILES: O=C(N1CCS(=O)(=O)CC1)N(Cc1ccc(-c2nnc(C(F)F)o2)cc1)c1ccccc1 ; O=S1(=O)CCN(C(=S)N(Cc2ccc(-c3nnc(C(F)F)o3)cc2)c2ccccc2)CC1]

N-((5-(5-(difluoromethyl)-1,3,4-oxadiazol-2-yl)pyridin-2-yl)methyl)-N-phenylthiomorpholin-4-carboxamide 1,1-dioxide (0.200 g, 0.432 mmol) and 2,4-bis(4-methoxyphenyl)-1,3,2,4-dithiadiphosphetan-2,4-disulfide (Lawesson's reagent, 0.175 g, 0.432 mmol) were dissolved in toluene (20 mL) at 110° C., after which the resulting solution was stirred at the same temperature for 18 hours to complete the reaction by lowering a temperature to room temperature. Water was poured into the reaction mixture and an organic layer was extracted with ethyl acetate. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 30%) and concentrated to obtain a title compound (0.027 g, 13.0%) as a yellow solid of a foam type.

¹H NMR (400 MHz, CDCl₃) δ 9.27 (d, J=2.0 Hz, 1H), 8.41 (dd, J=8.2, 2.2 Hz, 1H), 7.62 (d, J=8.2 Hz, 1H), 7.41 (t, J=7.9 Hz, 2H), 7.28-7.21 (m, 3H), 7.09 (s, 0.25H), 6.96 (s, 0.5H), 6.83 (s, 0.25H), 5.62 (s, 2H), 4.11˜4.06 (m, 4H), 2.97 (t, J=5.2 Hz, 4H); LRMS (ES) m/z 480.3 (M⁺+1).

### Example 7: Synthesis of compound 7, N-(4-(5-(difluoromethyl)-1,3,4-oxadiazol-2-yl)-2-fluorobenzyl)-4-methyl-N-phenylpiperazin-1-carbothioamide

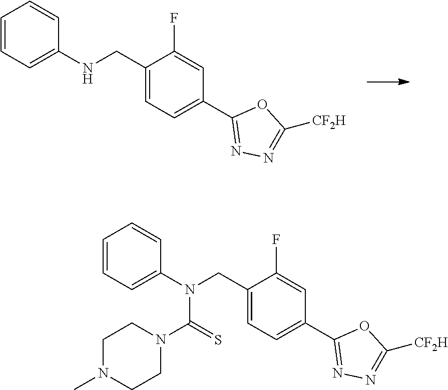 [SMILES: Fc1cc(-c2nnc(C(F)F)o2)ccc1CNc1ccccc1 ; CN1CCN(C(=S)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)aniline (0.200 g, 0.626 mmol) and N,N-diisopropylethylamine (0.218 mL, 1.253 mmol) were dissolved in dichloromethane (4 mL) at 0° C., after which thiophosgene (0.053 mL, 0.689 mmol) was added into the resulting solution and stirred at the same temperature. 1-methylpiperazine (0.084 mL, 0.752 mmol) was added into the reaction mixture and further stirred at room temperature for 18 hours. Saturated aqueous sodium chloride solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 2.5%) and concentrated to obtain a product, after which the resulting product was purified again via chromatography (SiO₂ plate, 20×20×1 mm; methanol/dichloromethane=3%) and concentrated to obtain a desired compound (0.034 g, 11.8%) as a yellow oil form.

¹H NMR (400 MHz, CDCl₃) δ 7.87 (d, J=1.4 Hz, 1H), 7.85-7.76 (m, 2H), 7.35-7.28 (m, 2H), 7.15-7.11 (m, 3H), 6.89 (t, J=51.7 Hz, 1H), 5.52 (s, 2H), 3.68 (t, J=5.0 Hz, 4H), 2.26 (t, J=5.0 Hz, 4H), 2.07 (s, 3H); LRMS (ES) m/z 462.3 (M⁺+1).

### Example 8: Synthesis of compound 8, N-(4-(5-(difluoromethyl)-1,3,4-oxadiazol-2-yl)-2-fluorobenzyl)-7-methyl-N-phenyl-7-azaspiro[3.5]nonan-2-carbothioamide

### [Step 1] Synthesis of N-(4-(5-(difluoromethyl)-1,3,4-oxadiazol-2-yl)-2-fluorobenzyl)-N-phenyl-7-azaspiro[3.5]nonan-2-carbothioamide

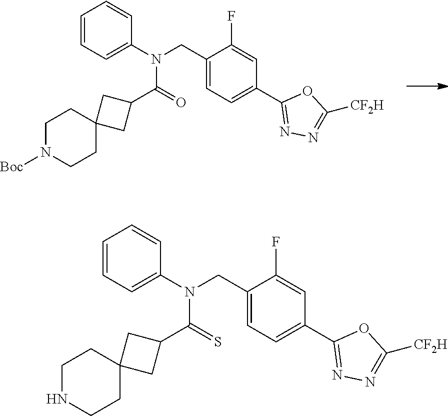 [SMILES: CC(C)(C)OC(=O)N1CCC2(CC1)CC(C(=O)N(Cc1ccc(-c3nnc(C(F)F)o3)cc1F)c1ccccc1)C2 ; Fc1cc(-c2nnc(C(F)F)o2)ccc1CN(C(=S)C1CC2(CCNCC2)C1)c1ccccc1]

Tert-butyl 2-((4-(5-(difluoromethyl)-1,3,4-oxadiazol-2-yl)-2-fluorobenzyl)(phenyl)carbamoyl)-7-azaspiro[3.5]nonan-7-carboxylate (0.110 g, 0.193 mmol) and 2,4-bis(4-methoxyphenyl)-1,3,2,4-dithiadiphosphetan-2,4-disulfide (Lawesson's reagent, 0.117 g, 0.289 mmol) were dissolved in toluene (10 mL) at 110° C., after which the resulting solution was stirred at the same temperature for 18 hours to complete the reaction by lowering a temperature to room temperature. Water was poured into the reaction mixture and an organic layer was extracted with ethyl acetate. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.077 g, 82.1%) as a brown oil form.

### [Step 2] Synthesis of Compound 8

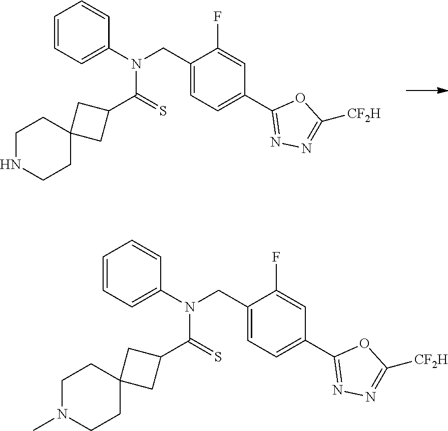 [SMILES: Fc1cc(-c2nnc(C(F)F)o2)ccc1CN(C(=S)C1CC2(CCNCC2)C1)c1ccccc1 ; CN1CCC2(CC1)CC(C(=S)N(Cc1ccc(-c3nnc(C(F)F)o3)cc1F)c1ccccc1)C2]

N-(4-(5-(difluoromethyl)-1,3,4-oxadiazol-2-yl)-2-fluorobenzyl)-N-phenyl-7-azaspiro[3.5]nonan-2-carbothioamide (0.077 g, 0.158 mmol) prepared in step 1, formaldehyde (0.010 g, 0.317 mmol) and sodium triacetoxyborohydride (0.067 g, 0.317 mmol) were dissolved in dichloromethane (10 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.035 g, 44.2%) as a white solid form.

¹H NMR (400 MHz, CDCl₃) 7.88 (d, J=8.o Hz, 1H), 7.73˜7.72 (m, 2H), 7.39˜7.38 (m, 3H), 7.05 (s, 0.25H), 6.98˜6.97 (m, 2H), 6.92 (s, 0.5H), 6.79 (s, 0.25H), 5.72 (s, 2H), 3.26˜3.22 (m, 1H), 3.10˜2.90 (m, 2H), 2.67 (s, 3H), 2.40˜2.24 (m, 2H), 2.06˜ 2.02 (m, 4H), 1.76˜1.74 (m, 4H); LRMS (ES) m/z 501.5 (M⁺+1).

### Example 9: Synthesis of compound 9, N-(4-(5-(difluoromethyl)-1,3,4-oxadiazol-2-yl)-2-fluorobenzyl)-N-phenylpyridin-4-carbothioamide

### [Step 1] Synthesis of N-(4-(2-(2,2-difluoroacetyl)hydrazin-1-carbonyl)-2-fluorobenzyl)-N-phenylpyridin-4-carbothioamide

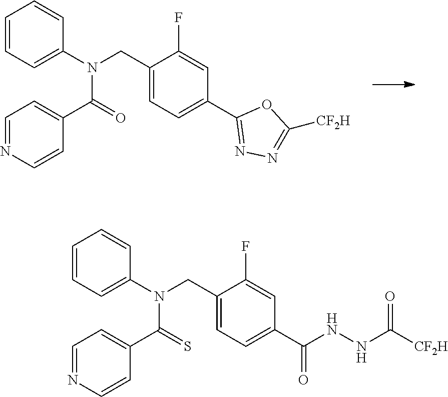 [SMILES: O=C(c1ccncc1)N(Cc1ccc(-c2nnc(C(F)F)o2)cc1F)c1ccccc1 ; O=C(NNC(=O)C(F)F)c1ccc(CN(C(=S)c2ccncc2)c2ccccc2)c(F)c1]

N-(4-(5-(difluoromethyl)-1,3,4-oxadiazol-2-yl)-2-fluorobenzyl)-N-phenylisonicotinamide (0.414 g, 0.976 mmol) and 2,4-bis(4-methoxyphenyl)-1,3,2,4-dithiadiphosphetan-2,4-disulfide (Lawesson's reagent, 0.592 g, 1.463 mmol) were dissolved in toluene (10 mL) at 110° C., after which the resulting solution was stirred at the same temperature for 18 hours to complete the reaction by lowering a temperature to room temperature. Water was poured into the reaction mixture and an organic layer was extracted with ethyl acetate. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.14 g, 31.3%) as a brown oil form.

### [Step 2] Synthesis of Compound 9

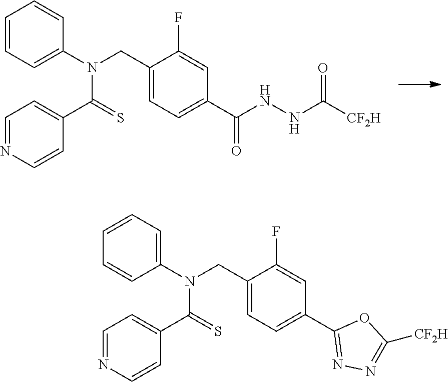 [SMILES: O=C(NNC(=O)C(F)F)c1ccc(CN(C(=S)c2ccncc2)c2ccccc2)c(F)c1 ; Fc1cc(-c2nnc(C(F)F)o2)ccc1CN(C(=S)c1ccncc1)c1ccccc1]

N-(4-(2-(2,2-difluoroacetyl)hydrazin-1-carbonyl)-2-fluorobenzyl)-N-phenylpyridin-4-carbothioamide (0.140 g, 0.305 mmol) prepared in step 1 and 1-methoxy-N-triethylammoniosulfonyl-methanimidate (Burgess reagent, 0.109 g, 0.458 mmol) were mixed in tetrahydrofuran (10 mL), irradiated with microwave, and heated at 150° C. for 30 minutes to complete the reaction by lowering a temperature to room temperature. Water was poured into the reaction mixture and an organic layer was extracted with ethyl acetate. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 40%) and concentrated to obtain a title compound (0.060 g, 44.6%) as a brown oil form.

¹H NMR (400 MHz, CDCl₃) δ 8.39 (d, J=5.8 Hz, 2H), 7.94˜7.71 (m, 3H), 7.20˜7.11 (m, 5H), 7.06 (s, 0.25H), 6.99˜6.94 (m, 2H), 6.94 (s, 0.5H), 6.80 (s, 0.25H), 5.88 (s, 2H); LRMS (ES) m/z 441.4 (M⁺+1).

### Example 10: Synthesis of compound 10, N-(4-(5-(difluoromethyl)-1,3,4-oxadiazol-2-yl)-2-fluorobenzyl)-6-methyl-N-phenyl-2,6-diazaspiro[3.3]heptan-2-carbothioamide

### [Step 1] Synthesis of tert-butyl 6-((4-(5-(difluoromethyl)-1,3,4-oxadiazol-2-yl)-2-fluorobenzyl)(phenyl)carbamothioyl)-2,6-diazaspiro[3.3]heptan-2-carboxylate

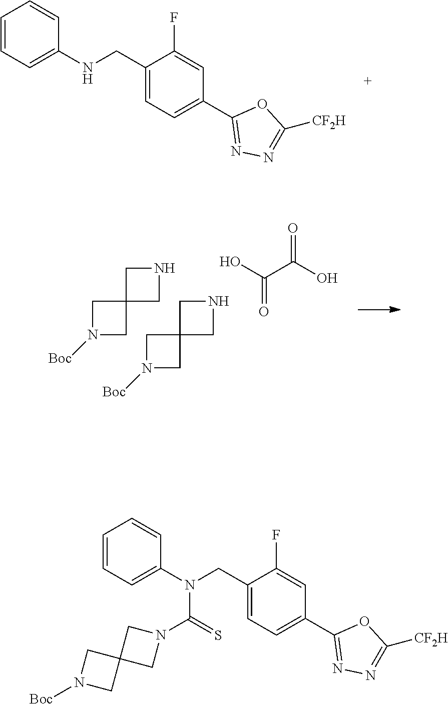

N-(4-(5-(difluoromethyl)-1,3,4-oxadiazol-2-yl)-2-fluorobenzyl)aniline (0.500 g, 1.566 mmol), tert-butyl 2,6-diazaspiro[3.3]heptan-2-carboxylate hemioxalate (0.457 g, 0.940 mmol), thiophosgene (0.132 mL, 1.723 mmol) and N,N-diisopropylethylamine (0.546 mL, 3.132 mmol) were dissolved in dichloromethane (5 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=10 to 70%) and concentrated to obtain a desired compound (0.433 g, 49.4%) as an orange oil form.

### [Step 2] Synthesis of N-(4-(5-(difluoromethyl)-1,3,4-oxadiazol-2-yl)-2-fluorobenzyl)-N-phenyl-2,6-diazaspiro[3.3]heptan-2-carbothioamide

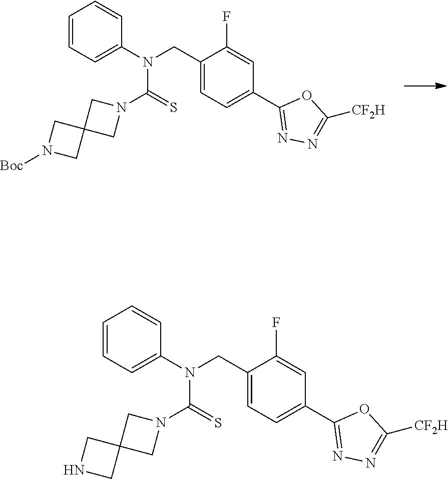 [SMILES: CC(C)(C)OC(=O)N1CC2(C1)CN(C(=S)N(Cc1ccc(-c3nnc(C(F)F)o3)cc1F)c1ccccc1)C2 ; Fc1cc(-c2nnc(C(F)F)o2)ccc1CN(C(=S)N1CC2(CNC2)C1)c1ccccc1]

Tert-butyl 6-((4-(5-(difluoromethyl)-1,3,4-oxadiazol-2-yl)-2-fluorobenzyl)(phenyl)carbamothioyl)-2,6-diazaspiro[3.3]heptan-2-carboxylate (0.433 g, 0.774 mmol) prepared in step 1 and trifluoroacetic acid (0.415 mL, 5.416 mmol) were dissolved in dichloromethane (5 mL) at room temperature, after which the resulting solution was stirred at the same temperature for five hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. An obtained product was used without a further purification process (0.340 g, 95.6%, yellow solid).

### [Step 3] Synthesis of Compound 10

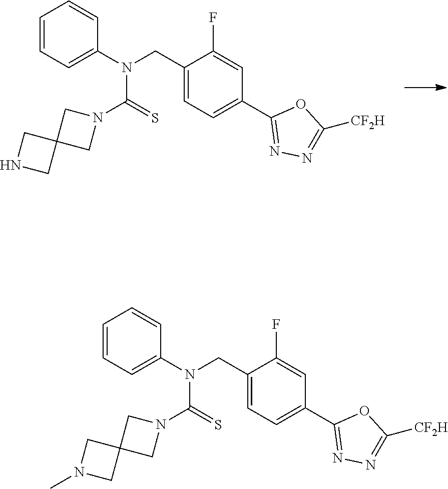 [SMILES: Fc1cc(-c2nnc(C(F)F)o2)ccc1CN(C(=S)N1CC2(CNC2)C1)c1ccccc1 ; CN1CC2(C1)CN(C(=S)N(Cc1ccc(-c3nnc(C(F)F)o3)cc1F)c1ccccc1)C2]

N-(4-(5-(difluoromethyl)-1,3,4-oxadiazol-2-yl)-2-fluorobenzyl)-N-phenyl-2,6-diazaspiro[3.3]heptan-2-carbothioamide (0.150 g, 0.326 mmol) prepared in step 2 and formaldehyde (38.00% solution, 0.036 mL, 0.490 mmol) were dissolved in dichloromethane (4 mL) at room temperature, after which sodium triacetoxyborohydride (0.138 g, 0.653 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 10%) and concentrated to obtain a desired compound (0.107 g, 69.2%) as a light yellow oil form.

¹H NMR (400 MHz, CDCl₃) δ 7.95 (t, J=7.6 Hz, 1H), 7.87 (dd, J=8.1, 1.5 Hz, 1H), 7.68 (dd, J=9.9, 1.5 Hz, 1H), 7.34-7.32 (m, 2H), 7.28-7.24 (m, 1H), 7.13-7.10 (m, 2H), 6.91 (t, J=51.7 Hz, 1H), 5.63 (s, 2H), 3.74 (brs, 4H), 3.18 (s, 4H), 2.22 (s, 3H); LRMS (ES) m/z 474.4 (M⁺+1).

### Example 11: Synthesis of compound 11, N-(4-(5-(difluoromethyl)-1,3,4-oxadiazol-2-yl)-2-fluorobenzyl)-6-(oxetan-3-yl)-N-phenyl-2,6-diazaspiro[3.3]heptan-2-carbothioamide

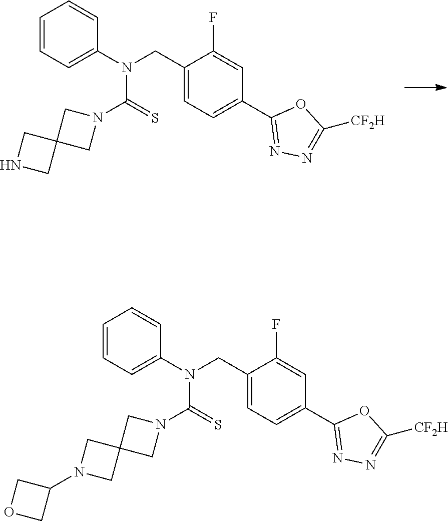 [SMILES: Fc1cc(-c2nnc(C(F)F)o2)ccc1CN(C(=S)N1CC2(CNC2)C1)c1ccccc1 ; Fc1cc(-c2nnc(C(F)F)o2)ccc1CN(C(=S)N1CC2(C1)CN(C1COC1)C2)c1ccccc1]

N-(4-(5-(difluoromethyl)-1,3,4-oxadiazol-2-yl)-2-fluorobenzyl)-N-phenyl-2,6-diazaspiro[3.3]heptan-2-carbothioamide (0.150 g, 0.326 mmol) prepared by the same method as described in step 2 of compound 10 and 3-oxetanone (0.029 mL, 0.490 mmol) were dissolved in dichloromethane (4 mL) at room temperature, after which sodium triacetoxyborohydride (0.138 g, 0.653 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 2.5%) and concentrated to obtain a product, after which the resulting product was purified again via chromatography (SiO₂, 4 g cartridge; ethyl acetate/hexane=50 to 100%) and concentrated to obtain a desired compound (0.062 g, 36.8%) as a light yellow solid form.

¹H NMR (400 MHz, CDCl₃) δ 7.94 (t, J=7.6 Hz, 1H), 7.87 (dd, J=8.1, 1.4 Hz, 1H), 7.67 (dd, J=9.9, 1.4 Hz, 1H), 7.35-7.31 (m, 2H), 7.29-7.26 (m, 1H), 7.13-7.11 (m, 2H), 6.91 (t, J=51.7 Hz, 1H), 5.63 (s, 2H), 4.63 (t, J=6.6 Hz, 2H), 4.37 (t, J=5.9 Hz, 2H), 3.84-3.80 (m, 5H), 3.26 (s, 4H); LRMS (ES) m/z 516.5 (M⁺+1).

### Example 12: Synthesis of compound 12, N-(4-(5-(difluoromethyl)-1,3,4-oxadiazol-2-yl)-2-fluorobenzyl)-N-(2,4-difluorophenyl)-6-methyl-2,6-diazaspiro[3.3]heptan-2-carbothioamide

### [Step 1] Synthesis of N-(4-(5-(difluoromethyl)-1,3,4-oxadiazol-2-yl)-2-fluorobenzyl)-2,4-difluoroaniline

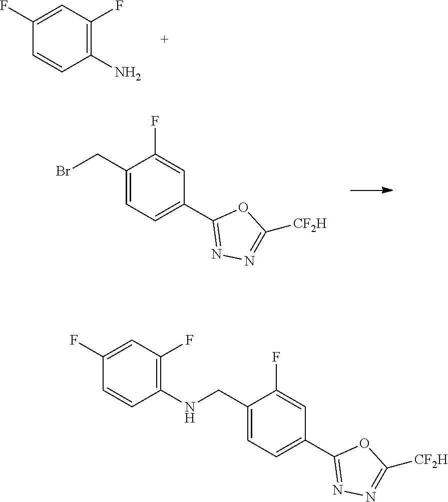 [SMILES: Nc1ccc(F)cc1F ; Fc1cc(-c2nnc(C(F)F)o2)ccc1CBr ; Fc1ccc(NCc2ccc(-c3nnc(C(F)F)o3)cc2F)c(F)c1]

2,4-difluoroaniline (0.500 g, 3.873 mmol), 2-(4-(bromomethyl)-3-fluorophenyl)-5-(difluoromethyl)-1,3,4-oxadiazole (1.189 g, 3.873 mmol) and potassium carbonate (1.070 g, 7.745 mmol) were dissolved in acetonitrile (20 mL) at 50° C., after which the resulting solution was stirred at the same temperature for 18 hours to complete the reaction by lowering a temperature to room temperature. Water was poured into the reaction mixture and an organic layer was extracted with ethyl acetate. The organic layer was washed with saturated aqueous sodium chloride solution, dehydrated with anhydrous sodium sulfate, filtered, and concentrated under reduced pressure. The resulting concentrate was purified via column chromatography (SiO₂, 40 g cartridge; ethyl acetate/hexane=0 to 30%) and concentrated to obtain a title compound (1.100 g, 80.0%) as a white solid form.

### [Step 2] Synthesis of tert-butyl 6-((4-(5-(difluoromethyl)-1,3,4-oxadiazol-2-yl)-2-fluorobenzyl)(2,4-difluorophenyl)carbamothioyl)-2,6-diazaspiro[3.3]heptan-2-carboxylate

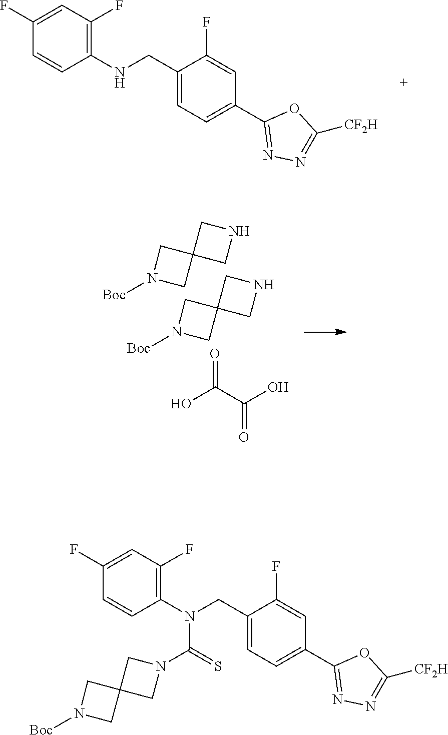

N-(4-(5-(difluoromethyl)-1,3,4-oxadiazol-2-yl)-2-fluorobenzyl)-2,4-difluoroaniline (0.843 g, 2.373 mmol) prepared in step 1, N,N-diisopropylethylamine (1.653 mL, 9.491 mmol) and thiophosgene (0.704 g, 2.373 mmol) were dissolved in dichloromethane (20 mL), after which the resulting solution was stirred at 0° C. for 30 minutes and then tert-butyl 2,6-diazaspiro[3.3]heptan-2-carboxylate hemioxalate (0.577 g, 1.186 mmol) was added thereinto and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 50%) and concentrated to obtain a title compound (0.200 g, 14.2%) as a colorless oil form.

### [Step 3] Synthesis of N-(4-(5-(difluoromethyl)-1,3,4-oxadiazol-2-yl)-2-fluorobenzyl)-N-(2,4-difluorophenyl)-2,6-diazaspiro[3.3]heptan-2-carbothioamide 2,2,2-trifluoroacetate

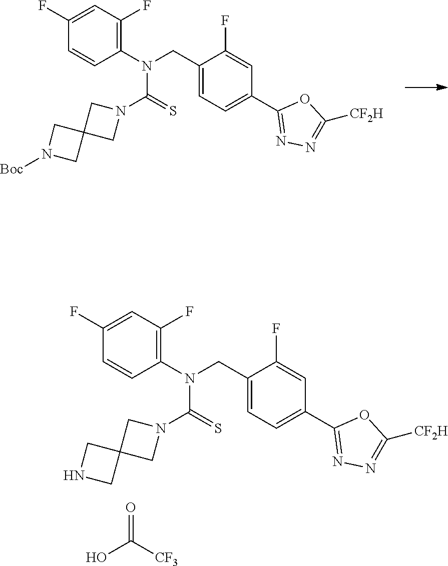

Tert-butyl 6-((4-(5-(difluoromethyl)-1,3,4-oxadiazol-2-yl)-2-fluorobenzyl)(2,4-difluorophenyl)carbamothioyl)-2,6-diazaspiro[3.3]heptan-2-carboxylate (0.084 g, 0.141 mmol) prepared in step 2 was dissolved in dichloromethane (10 mL) at room temperature, after which the resulting solution was stirred at the same temperature for 18 hours. Solvent was removed from the reaction mixture under reduced pressure, after which a product obtained was used without a further purification process (0.084 g, 97.7%, yellow oil).

### [Step 4] Synthesis of Compound 12

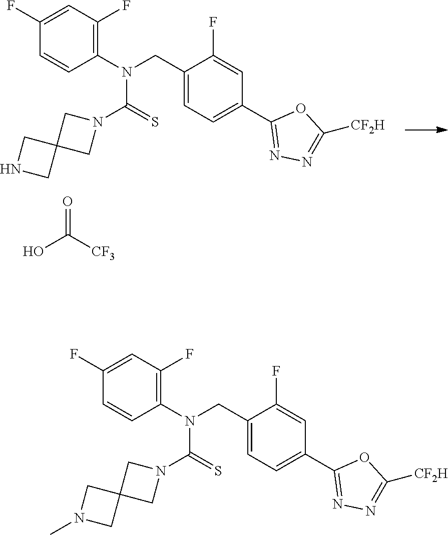

N-(4-(5-(difluoromethyl)-1,3,4-oxadiazol-2-yl)-2-fluorobenzyl)-N-(2,4-difluorophenyl)-2,6-diazaspiro[3.3]heptan-2-carbothioamide 2,2,2-trifluoroacetate (0.084 g, 0.138 mmol) prepared in step 3, N,N-diisopropylethylamine (0.024 mL, 0.138 mmol), sodium triacetoxyborohydride (0.058 g, 0.276 mmol) and formaldehyde (0.008 g, 0.276 mmol) were dissolved in dichloromethane (10 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 30%) and concentrated to obtain a title compound (0.020 g, 28.5%) as a yellow oil form.

¹H NMR (400 MHz, CDCl₃) δ 8.01 (t, J=7.6 Hz, 1H), 7.87 (dd, J=8.1, 1.2 Hz, 1H), 7.67 (dd, J=9.9, 1.2 Hz, 1H), 7.07˜7.01 (m, 1H), 7.04 (s, 0.25H), 6.92 (s, 0.5H), 6.92˜6.82 (m, 2H), 6.79 (s, 0.25H), 5.55 (s, 2H), 3.84 (s, 4H), 3.41 (s, 4H), 2.34 (s, 3H); LRMS (ES) m/z 510.5 (M⁺+1).

### Example 13: Synthesis of compound 13, N-(4-(5-(difluoromethyl)-1,3,4-oxadiazol-2-yl)-2-fluorobenzyl)-N-(3,4-difluorophenyl)-6-methyl-2,6-diazaspiro[3.3]heptan-2-carbothioamide

### [Step 1] Synthesis of N-(4-(5-(difluoromethyl)-1,3,4-oxadiazol-2-yl)-2-fluorobenzyl)-3,4-difluoroaniline

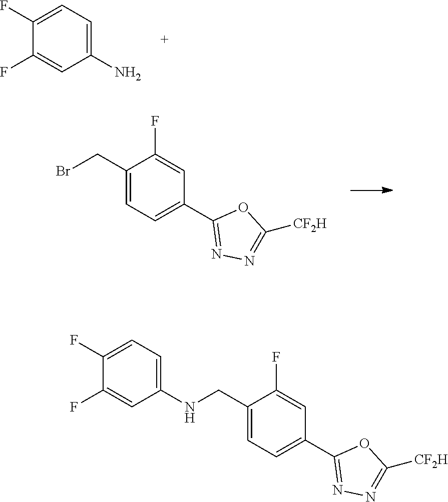 [SMILES: Nc1ccc(F)c(F)c1 ; Fc1cc(-c2nnc(C(F)F)o2)ccc1CBr ; Fc1ccc(NCc2ccc(-c3nnc(C(F)F)o3)cc2F)cc1F]

3,4-difluoroaniline (0.500 g, 3.873 mmol), 2-(4-(bromomethyl)-3-fluorophenyl)-5-(difluoromethyl)-1,3,4-oxadiazole (1.189 g, 3.873 mmol) and potassium carbonate (1.070 g, 7.745 mmol) were dissolved in acetonitrile (20 mL) at 50° C., after which the resulting solution was stirred at the same temperature for 18 hours to complete the reaction by lowering a temperature to room temperature. Water was poured into the reaction mixture and an organic layer was extracted with ethyl acetate. The organic layer was washed with saturated aqueous sodium chloride solution, dehydrated with anhydrous sodium sulfate, filtered, and concentrated under reduced pressure. The resulting concentrate was purified via column chromatography (SiO₂, 40 g cartridge; ethyl acetate/hexane=0 to 30%) and concentrated to obtain a title compound (0.880 g, 64.0%) as a white solid form.

### [Step 2] Synthesis of tert-butyl 6-((4-(5-(difluoromethyl)-1,3,4-oxadiazol-2-yl)-2-fluorobenzyl)(3,4-difluorophenyl)carbamothioyl)-2,6-diazaspiro[3.3]heptan-2-carboxylate

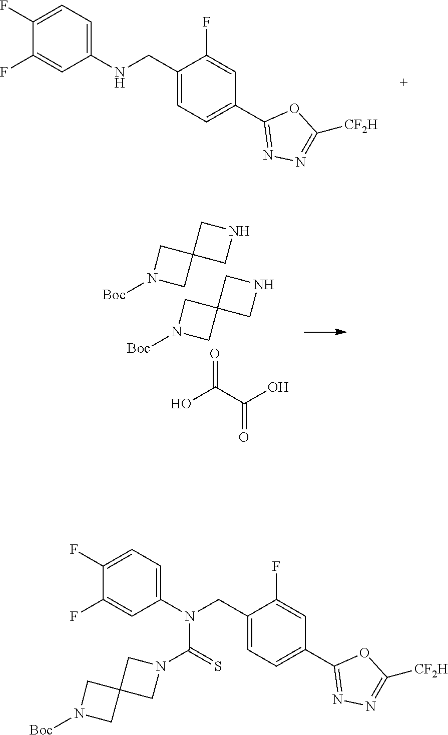

N-(4-(5-(difluoromethyl)-1,3,4-oxadiazol-2-yl)-2-fluorobenzyl)-3,4-difluoroaniline (0.756 g, 2.128 mmol) prepared in step 1, N,N-diisopropylethylamine (1.483 mL, 8.512 mmol) and thiophosgene (0.631 g, 2.128 mmol) were dissolved in dichloromethane (20 mL), after which the resulting solution was stirred at 0° C. for 30 minutes and then tert-butyl 2,6-diazaspiro[3.3]heptan-2-carboxylate hemioxalate (0.518 g, 1.064 mmol) was added thereinto and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 50%) and concentrated to obtain a title compound (0.200 g, 15.8%) as a colorless oil form.

### [Step 3] Synthesis of N-(4-(5-(difluoromethyl)-1,3,4-oxadiazol-2-yl)-2-fluorobenzyl)-N-(3,4-difluorophenyl)-2,6-diazaspiro[3.3]heptan-2-carbothioamide 2,2,2-trifluoroacetate

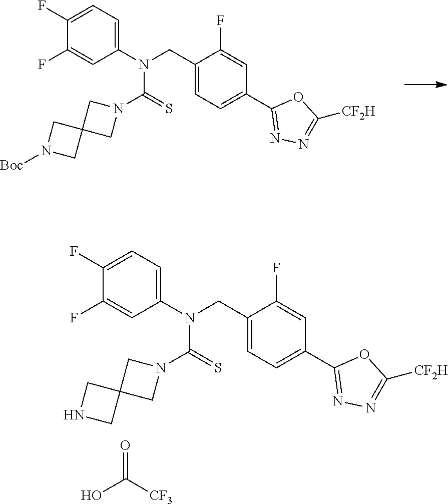

Tert-butyl 6-((4-(5-(difluoromethyl)-1,3,4-oxadiazol-2-yl)-2-fluorobenzyl)(3,4-difluorophenyl)carbamothioyl)-2,6-diazaspiro[3.3]heptan-2-carboxylate (0.140 g, 0.235 mmol) prepared in step 2 and trifluoroacetic acid (0.180 mL, 2.351 mmol) were dissolved in dichloromethane (10 mL) at room temperature, after which the resulting solution was stirred at the same temperature for 18 hours. Solvent was removed from the reaction mixture under reduced pressure, after which a product obtained was used without a further purification process (0.140 g, 97.7%, yellow oil).

### [Step 4] Synthesis of Compound 13

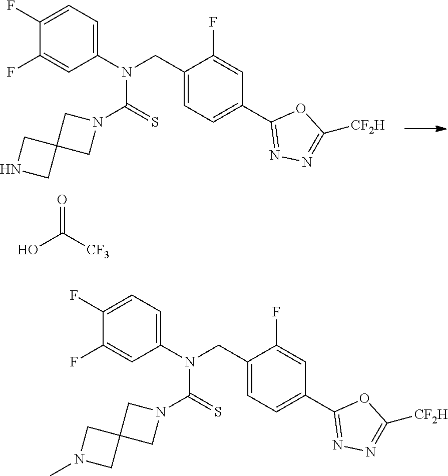

N-(4-(5-(difluoromethyl)-1,3,4-oxadiazol-2-yl)-2-fluorobenzyl)-N-(3,4-difluorophenyl)-2,6-diazaspiro[3.3]heptan-2-carbothioamide 2,2,2-trifluoroacetate (0.140 g, 0.230 mmol) prepared in step 3, N,N-diisopropylethylamine (0.040 mL, 0.230 mmol), sodium triacetoxyborohydride (0.097 g, 0.459 mmol) and formaldehyde (0.014 g, 0.459 mmol) were dissolved in dichloromethane (10 mL) at room temperature, after which the resulting solution was stirred at the same temperature for 18 hours. Water was poured into the reaction mixture and an organic layer was extracted with dichloromethane. The organic layer was washed with saturated sodium chloride aqueous solution, dehydrated with anhydrous sodium sulfate, filtered, and concentrated under reduced pressure. The resulting concentrate was purified via column chromatography (SiO₂, 12 g cartridge; methanol/dichloromethane=0 to 30%) and concentrated to obtain a title compound (0.060 g, 51.3%) as a yellow oil form.

¹H NMR (400 MHz, CDCl₃) δ 7.93˜7.88 (m, 2H), 7.72 (d, J=10.2 Hz, 1H), 7.14 (dd, J=18.0, 8.9 Hz, 1H), 7.05 (s, 0.25H), 7.01˜6.96 (m, 1H), 6.94 (s, 0.5H), 6.88˜6.86 (m, 1H), 6.79 (s, 0.25H), 5.56 (s, 2H), 4.00˜3.70 (m, 4H), 3.36 (s, 4H), 2.36 (s, 3H); LRMS (ES) m/z 510.5 (M⁺+1).

### Example 14: Synthesis of compound 14, N-(4-(5-(difluoromethyl)-1,3,4-oxadiazol-2-yl)benzyl)-N-(3-fluorophenyl)-6-methyl-2,6-diazaspiro[3.3]heptan-2-carbothioamide

### [Step 1] Synthesis of N-(4-(5-(difluoromethyl)-1,3,4-oxadiazol-2-yl)benzyl)-N-(3-fluorophenyl)-2,6-diazaspiro[3.3]heptan-2-carbothioamide

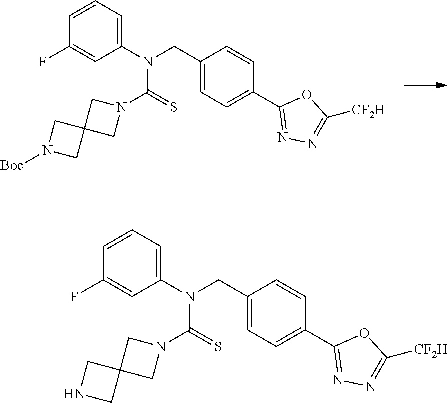 [SMILES: CC(C)(C)OC(=O)N1CC2(C1)CN(C(=S)N(Cc1ccc(-c3nnc(C(F)F)o3)cc1)c1cccc(F)c1)C2 ; Fc1cccc(N(Cc2ccc(-c3nnc(C(F)F)o3)cc2)C(=S)N2CC3(CNC3)C2)c1]

Tert-butyl 6-((4-(5-(difluoromethyl)-1,3,4-oxadiazol-2-yl)benzyl)(3-fluorophenyl)carbamothioyl)-2,6-diazaspiro[3.3]heptan-2-carboxylate (0.500 g, 0.893 mmol) and trifluoroacetic acid (0.479 mL, 6.254 mmol) were dissolved in dichloromethane (5 mL) at room temperature, after which the resulting solution was stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. An obtained product was used without a further purification process (0.361 g, 93.7%, yellow solid).

### [Step 2] Synthesis of Compound 14

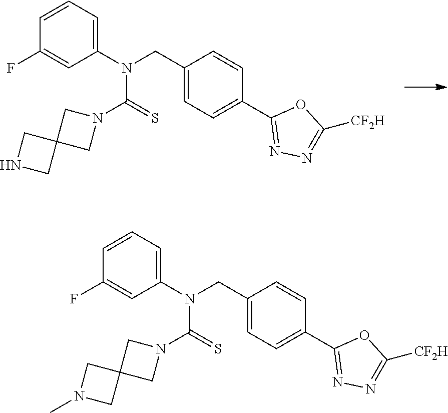 [SMILES: Fc1cccc(N(Cc2ccc(-c3nnc(C(F)F)o3)cc2)C(=S)N2CC3(CNC3)C2)c1 ; CN1CC2(C1)CN(C(=S)N(Cc1ccc(-c3nnc(C(F)F)o3)cc1)c1cccc(F)c1)C2]

N-(4-(5-(difluoromethyl)-1,3,4-oxadiazol-2-yl)benzyl)-N-(3-fluorophenyl)-2,6-diazaspiro[3.3]heptan-2-carbothioamide (0.100 g, 0.218 mmol) prepared in step 1 and formaldehyde (38.00% solution, 0.024 mL, 0.326 mmol) were dissolved in dichloromethane (4 mL) at room temperature, after which sodium triacetoxyborohydride (0.092 g, 0.435 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a desired compound (0.038 g, 36.9%) as a white solid form.

¹H NMR (400 MHz, CDCl₃) δ 8.04 (d, J=8.2 Hz, 2H), 7.54 (d, J=8.2 Hz, 2H), 7.32-7.26 (m, 1H), 7.05-6.79 (m, 4H), 5.55 (s, 2H), 3.83 (brs, 4H), 3.25 (s, 4H), 2.27 (s, 3H); LRMS (ES) m/z 474.7 (M⁺+1).

### Example 15: Synthesis of compound 15, N-(4-(5-(difluoromethyl)-1,3,4-oxadiazol-2-yl)benzyl)-N-(3-fluorophenyl)-6-(oxetan-3-yl)-2,6-diazaspiro[3.3]heptan-2-carbothioamide

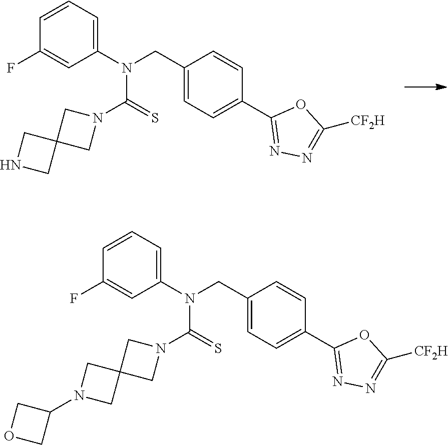 [SMILES: Fc1cccc(N(Cc2ccc(-c3nnc(C(F)F)o3)cc2)C(=S)N2CC3(CNC3)C2)c1 ; Fc1cccc(N(Cc2ccc(-c3nnc(C(F)F)o3)cc2)C(=S)N2CC3(C2)CN(C2COC2)C3)c1]

N-(4-(5-(difluoromethyl)-1,3,4-oxadiazol-2-yl)benzyl)-N-(3-fluorophenyl)-2,6-diazaspiro[3.3]heptan-2-carbothioamide (0.100 g, 0.218 mmol) prepared by the same method as described in step 1 of compound 14 and 3-oxetanone (0.021 mL, 0.326 mmol) were dissolved in dichloromethane (4 mL) at room temperature, after which sodium triacetoxyborohydride (0.092 g, 0.435 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 2.5%) and concentrated to obtain a desired compound (0.046 g, 41.0%) as a light yellow solid form.

¹H NMR (400 MHz, CDCl₃) δ 8.03 (d, J=8.2 Hz, 2H), 7.53 (d, J=8.2 Hz, 2H), 7.33-7.27 (m, 1H), 7.05-6.79 (m, 4H), 5.55 (s, 2H), 4.65 (t, J=6.7 Hz, 2H), 4.40 (t, J=5.9 Hz, 2H), 3.87 (brs, 4H), 3.66-3.63 (m, 1H), 3.30 (s, 4H); LRMS (ES) m/z 516.7 (M⁺+1).

### Example 16: Synthesis of compound 16, N-(4-(5-(difluoromethyl)-1,3,4-oxadiazol-2-yl)benzyl)-N-(3-fluorophenyl)-6-isopropyl-2,6-diazaspiro[3.3]heptan-2-carbothioamide

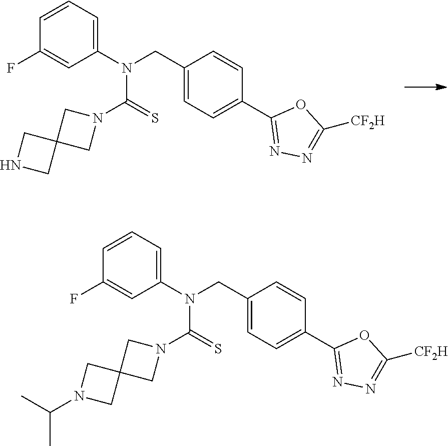 [SMILES: Fc1cccc(N(Cc2ccc(-c3nnc(C(F)F)o3)cc2)C(=S)N2CC3(CNC3)C2)c1 ; CC(C)N1CC2(CN(C(=S)N(Cc3ccc(-c4nnc(C(F)F)o4)cc3)c3cccc(F)c3)C2)C1]

N-(4-(5-(difluoromethyl)-1,3,4-oxadiazol-2-yl)benzyl)-N-(3-fluorophenyl)-2,6-diazaspiro[3.3]heptan-2-carbothioamide (0.100 g, 0.218 mmol) prepared by the same method as described in step 1 of compound 14 and acetone (0.024 mL, 0.326 mmol) were dissolved in dichloromethane (4 mL) at room temperature, after which sodium triacetoxyborohydride (0.092 g, 0.435 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; methanol/dichloromethane=0 to 5%) and concentrated to obtain a desired compound (0.028 g, 25.7%) as a white solid form.

¹H NMR (400 MHz, CDCl₃) δ 8.03 (d, J=8.2 Hz, 2H), 7.54 (d, J=8.1 Hz, 2H), 7.31-7.26 (m, 1H), 7.05-6.79 (m, 4H), 5.55 (s, 2H), 3.83 (brs, 4H), 3.22 (s, 4H), 2.23-2.15 (m, 1H), 0.90 (d, J=6.0 Hz, 6H); LRMS (ES) m/z 502.7 (M⁺+1).

### Example 17: Synthesis of compound 17, N-(4-(5-(difluoromethyl)-1,3,4-oxadiazol-2-yl)-2-fluorobenzyl)-N-(4-fluorophenyl)-6-methyl-2,6-diazaspiro[3.3]heptan-2-carbothioamide

### [Step 1] Synthesis of N-(4-(5-(difluoromethyl)-1,3,4-oxadiazol-2-yl)-2-fluorobenzyl)-4-fluoroaniline

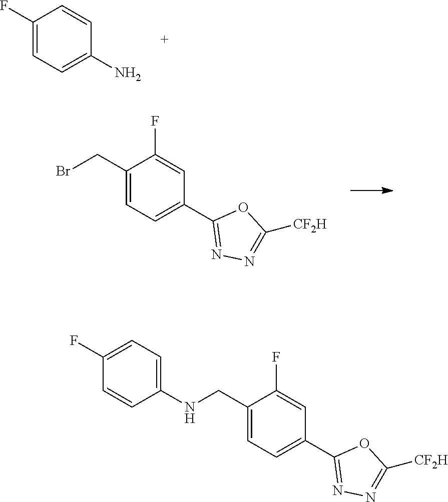 [SMILES: Nc1ccc(F)cc1 ; Fc1cc(-c2nnc(C(F)F)o2)ccc1CBr ; Fc1ccc(NCc2ccc(-c3nnc(C(F)F)o3)cc2F)cc1]

4-fluoroaniline (1.000 g, 8.999 mmol) and sodium hydride (60.00%, 0.378 g, 9.449 mmol) were dissolved in N,N-dimethylformamide (30 mL) at 0° C., after which 2-(4-(bromomethyl)-3-fluorophenyl)-5-(difluoromethyl)-1,3,4-oxadiazole (2.902 g, 9.449 mmol) was added into the resulting solution and stirred at room temperature for 18 hours. Solvent was removed from the reaction mixture under reduced pressure, after which saturated aqueous sodium hydrogen carbonate solution was poured into the resulting concentrate and an organic layer was extracted with ethyl acetate. The organic layer was washed with saturated aqueous sodium chloride solution, dehydrated with anhydrous magnesium sulfate, filtered, and concentrated under reduced pressure. The resulting concentrate was purified via column chromatography (SiO₂, 24 g cartridge; ethyl acetate/hexane=5 to 20%) and concentrated to obtain a desired compound (1.360 g, 44.8%) as a yellow solid form.

### [Step 2] Synthesis of tert-butyl 6-((4-(5-(difluoromethyl)-1,3,4-oxadiazol-2-yl)-2-fluorobenzyl)(4-fluorophenyl)carbamothioyl)-2,6-diazaspiro[3.3]heptan-2-carboxylate

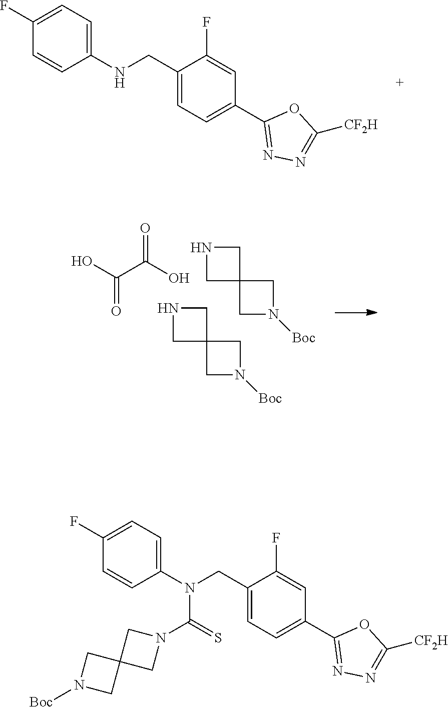

N-(4-(5-(difluoromethyl)-1,3,4-oxadiazol-2-yl)-2-fluorobenzyl)-4-fluoroaniline (1.000 g, 2.965 mmol) prepared in step 1 and N,N-diisopropylethylamine (1.549 mL, 8.895 mmol) were dissolved in dichloromethane (30 mL) at 0° C., after which thiophosgene (0.227 mL, 2.965 mmol) was added into the resulting solution and stirred at the same temperature. Tert-butyl 2,6-diazaspiro[3.3]heptan-2-carboxylate hemioxalate (0.866 g, 1.779 mmol) was added into the reaction mixture and further stirred at room temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; ethyl acetate/hexane=10 to 30%) and concentrated to obtain a desired compound (1.220 g, 71.2%) as a light yellow solid form.

### [Step 3] Synthesis of N-(4-(5-(difluoromethyl)-1,3,4-oxadiazol-2-yl)-2-fluorobenzyl)-N-(4-fluorophenyl)-2,6-diazaspiro[3.3]heptan-2-carbothioamide

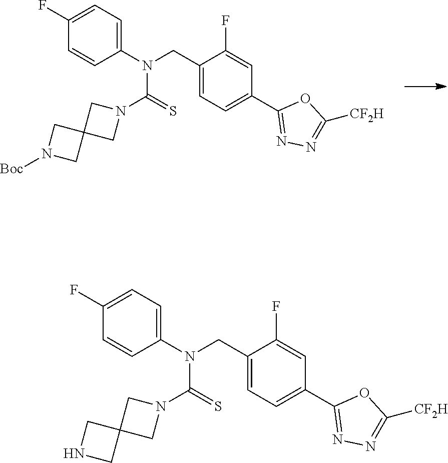 [SMILES: CC(C)(C)OC(=O)N1CC2(C1)CN(C(=S)N(Cc1ccc(-c3nnc(C(F)F)o3)cc1F)c1ccc(F)cc1)C2 ; Fc1ccc(N(Cc2ccc(-c3nnc(C(F)F)o3)cc2F)C(=S)N2CC3(CNC3)C2)cc1]

Tert-butyl 6-((4-(5-(difluoromethyl)-1,3,4-oxadiazol-2-yl)-2-fluorobenzyl)(4-fluorophenyl)carbamothioyl)-2,6-diazaspiro[3.3]heptan-2-carboxylate (1.220 g, 2.112 mmol) prepared in step 2 and trifluoroacetic acid (1.132 mL, 14.785 mmol) were dissolved in dichloromethane (50 mL) at room temperature, after which the resulting solution was stirred at the same temperature for 18 hours. Saturated aqueous sodium hydrogen carbonate solution was poured into the reaction mixture and an organic layer was extracted with dichloromethane. The organic layer was washed with saturated aqueous sodium chloride solution, dehydrated with anhydrous magnesium sulfate, filtered, and concentrated under reduced pressure. An obtained product was used without a further purification process (0.964 g, 95.6%, light yellow solid).

### [Step 4] Synthesis of Compound 17

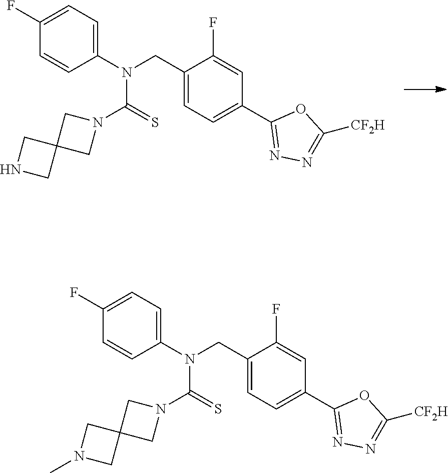 [SMILES: Fc1ccc(N(Cc2ccc(-c3nnc(C(F)F)o3)cc2F)C(=S)N2CC3(CNC3)C2)cc1 ; CN1CC2(C1)CN(C(=S)N(Cc1ccc(-c3nnc(C(F)F)o3)cc1F)c1ccc(F)cc1)C2]

N-(4-(5-(difluoromethyl)-1,3,4-oxadiazol-2-yl)-2-fluorobenzyl)-N-(4-fluorophenyl)-2,6-diazaspiro[3.3]heptan-2-carbothioamide (0.100 g, 0.209 mmol) prepared in step 3 and formaldehyde (38.00% solution, 0.023 mL, 0.314 mmol) were dissolved in dichloromethane (4 mL) at room temperature, after which sodium triacetoxyborohydride (0.089 g, 0.419 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a desired compound (0.037, 35.9%) as a white solid form.

¹H NMR (400 MHz, CDCl₃) δ 7.95 (t, J=7.5 Hz, 1H), 7.88 (d, J=8.1 Hz, 1H), 7.68 (d, J=9.9 Hz, 1H), 7.10-7.08 (m, 2H), 7.07-6.79 (m, 3H), 5.60 (s, 2H), 3.78 (brs, 4H), 3.20 (s, 4H), 2.23 (s, 3H); LRMS (ES) m/z 492.7 (M⁺+1).

### Example 18: Synthesis of compound 18, N-(4-(5-(difluoromethyl)-1,3,4-oxadiazol-2-yl)-2-fluorobenzyl)-N-(4-fluorophenyl)-6-isopropyl-2,6-diazaspiro[3.3]heptan-2-carbothioamide

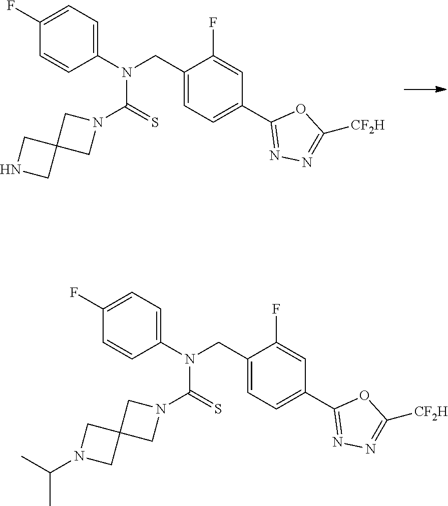 [SMILES: Fc1ccc(N(Cc2ccc(-c3nnc(C(F)F)o3)cc2F)C(=S)N2CC3(CNC3)C2)cc1 ; CC(C)N1CC2(CN(C(=S)N(Cc3ccc(-c4nnc(C(F)F)o4)cc3F)c3ccc(F)cc3)C2)C1]

N-(4-(5-(difluoromethyl)-1,3,4-oxadiazol-2-yl)-2-fluorobenzyl)-N-(4-fluorophenyl)-2,6-diazaspiro[3.3]heptan-2-carbothioamide (0.100 g, 0.209 mmol) prepared by the same method as described in step 3 of compound 17 and acetone (0.023 mL, 0.314 mmol) were dissolved in dichloromethane (4 mL) at room temperature, after which sodium triacetoxyborohydride (0.089 g, 0.419 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a desired compound (0.030 g, 27.6%) as a light yellow solid form.

¹H NMR (400 MHz, CDCl₃) δ 7.94 (t, J=7.6 Hz, 1H), 7.87 (d, J=8.1 Hz, 1H), 7.68 (d, J=9.9 Hz, 1H), 7.10-7.06 (m, 2H), 7.02-6.79 (m, 3H), 5.59 (s, 2H), 3.72 (brs, 4H), 3.19 (s, 4H), 2.20-2.17 (m, 1H), 0.86 (d, J=6.2 Hz, 6H); LRMS (ES) m/z 520.7 (M⁺+1).

### Example 19: Synthesis of compound 19, N-(4-(5-(difluoromethyl)-1,3,4-oxadiazol-2-yl)-2-fluorobenzyl)-N-(4-fluorophenyl)-6-(oxetan-3-yl)-2,6-diazaspiro[3.3]heptan-2-carbothioamide

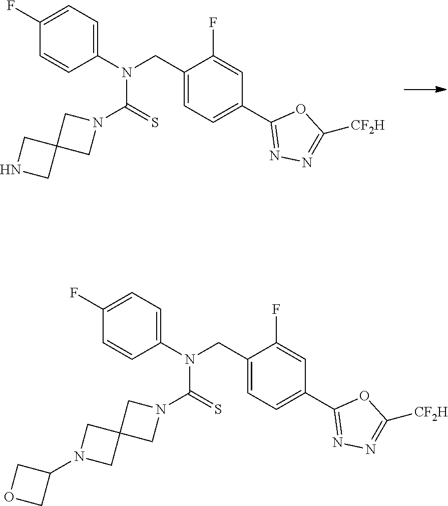 [SMILES: Fc1ccc(N(Cc2ccc(-c3nnc(C(F)F)o3)cc2F)C(=S)N2CC3(CNC3)C2)cc1 ; Fc1ccc(N(Cc2ccc(-c3nnc(C(F)F)o3)cc2F)C(=S)N2CC3(C2)CN(C2COC2)C3)cc1]

N-(4-(5-(difluoromethyl)-1,3,4-oxadiazol-2-yl)-2-fluorobenzyl)-N-(4-fluorophenyl)-2,6-diazaspiro[3.3]heptan-2-carbothioamide (0.100 g, 0.209 mmol) prepared by the same method as described in step 3 of compound 17 and 3-oxetanone (0.020 mL, 0.314 mmol) were dissolved in dichloromethane (4 mL) at room temperature, after which sodium triacetoxyborohydride (0.089 g, 0.419 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 2.5%) and concentrated to obtain a desired compound (0.016 g, 14.3%) as a white solid form.

¹H NMR (400 MHz, CDCl₃) δ 7.96 (t, J=7.6 Hz, 1H), 7.88 (d, J=8.1 Hz, 1H), 7.69 (d, J=9.9 Hz, 1H), 7.12-7.08 (m, 2H), 7.04 (d, J=8.1 Hz, 2H), 7.01-6.79 (m, 1H), 5.60 (s, 2H), 4.64 (t, J=6.6 Hz, 2H), 4.39 (t, J=5.9 Hz, 2H), 3.83 (brs, 4H), 3.75-3.62 (m, 1H), 3.27 (s, 4H); LRMS (ES) m/z 534.6 (M⁺+1).

### Example 20: Synthesis of compound 20, N-(4-(5-(difluoromethyl)-1,3,4-oxadiazol-2-yl)benzyl)-N-(4-fluorophenyl)-6-methyl-2,6-diazaspiro[3.3]heptan-2-carbothioamide

### [Step 1] Synthesis of N-(4-(5-(difluoromethyl)-1,3,4-oxadiazol-2-yl)benzyl)-4-fluoroaniline

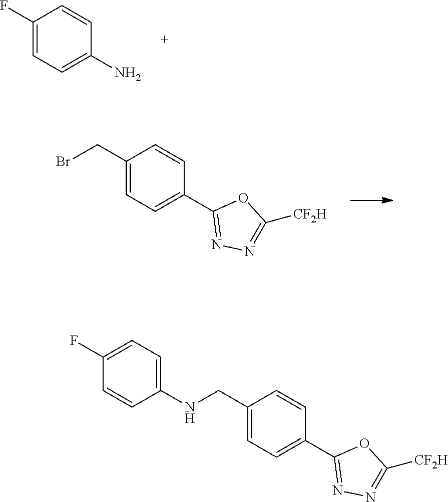 [SMILES: Nc1ccc(F)cc1 ; FC(F)c1nnc(-c2ccc(CBr)cc2)o1 ; Fc1ccc(NCc2ccc(-c3nnc(C(F)F)o3)cc2)cc1]

4-fluoroaniline (1.000 g, 8.999 mmol) and sodium hydride (60.00%, 0.378 g, 9.449 mmol) were dissolved in N,N-dimethylformamide (30 mL) at 0° C., after which 2-(4-(bromomethyl)phenyl)-5-(difluoromethyl)-1,3,4-oxadiazole (2.732 g, 9.449 mmol) was added into the resulting solution and stirred at room temperature for 18 hours. Solvent was removed from the reaction mixture under reduced pressure, after which saturated aqueous sodium hydrogen carbonate solution was poured into the resulting concentrate and an organic layer was extracted with ethyl acetate. The organic layer was washed with saturated aqueous sodium chloride solution, dehydrated with anhydrous magnesium sulfate, filtered, and concentrated under reduced pressure. The resulting concentrate was purified via column chromatography (SiO₂, 24 g cartridge; ethyl acetate/hexane=5 to 20%) and concentrated to obtain a desired compound (1.510 g, 52.6%) as a pink solid form.

### [Step 2] Synthesis of tert-butyl 6-((4-(5-(difluoromethyl)-1,3,4-oxadiazol-2-yl)benzyl)(4-fluorophenyl)carbamothioyl)-2,6-diazaspiro[3.3]heptan-2-carboxylate

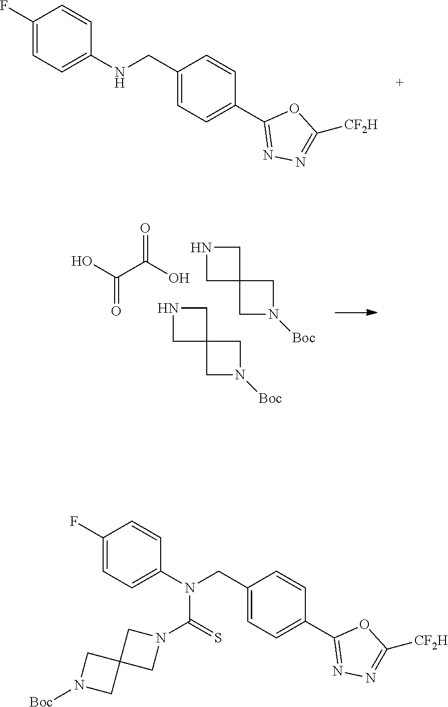

N-(4-(5-(difluoromethyl)-1,3,4-oxadiazol-2-yl)benzyl)-4-fluoroaniline (1.ooo g, 3.132 mmol) prepared in step 1 and N,N-diisopropylethylamine (1.637 mL, 9.396 mmol) were dissolved in dichloromethane (50 mL) at 0° C., after which thiophosgene (0.360 g, 3.132 mmol) was added into the resulting solution and stirred at the same temperature. Tert-butyl 2,6-diazaspiro[3.3]heptan-2-carboxylate hemioxalate (0.914 g, 1.879 mmol) was added into the reaction mixture and further stirred at room temperature for 18 hours. Aqueous N-sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; ethyl acetate/hexane=10 to 40%) and concentrated to obtain a desired compound (1.200 g, 68.5%) as a yellow solid form.

### [Step 3] Synthesis of N-(4-(5-(difluoromethyl)-1,3,4-oxadiazol-2-yl)benzyl)-N-(4-fluorophenyl)-2,6-diazaspiro[3.3]heptan-2-carbothioamide

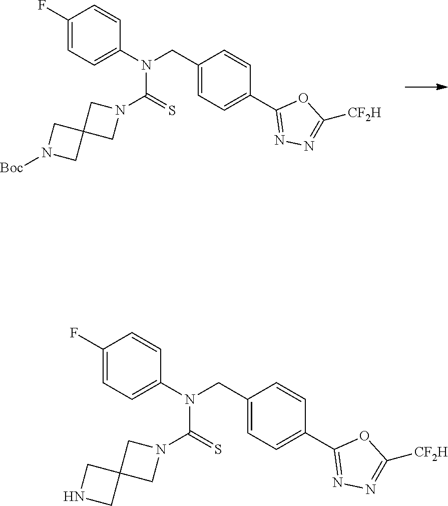 [SMILES: CC(C)(C)OC(=O)N1CC2(C1)CN(C(=S)N(Cc1ccc(-c3nnc(C(F)F)o3)cc1)c1ccc(F)cc1)C2 ; Fc1ccc(N(Cc2ccc(-c3nnc(C(F)F)o3)cc2)C(=S)N2CC3(CNC3)C2)cc1]

Tert-butyl 6-((4-(5-(difluoromethyl)-1,3,4-oxadiazol-2-yl)benzyl)(4-fluorophenyl)carbamothioyl)-2,6-diazaspiro[3.3]heptan-2-carboxylate (1.200 g, 2.144 mmol) prepared by the same method as described in step 2 and trifluoroacetic acid (1.149 mL, 15.010 mmol) were dissolved in dichloromethane (15 mL) at room temperature, after which the resulting solution was stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. An obtained product was used without a further purification process (0.948 g, 96.2%, light yellow solid).

### [Step 4] Synthesis of Compound 20

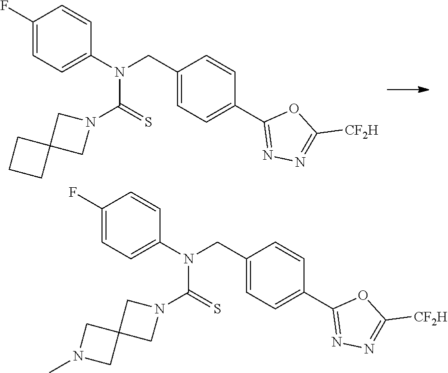

N-(4-(5-(difluoromethyl)-1,3,4-oxadiazol-2-yl)benzyl)-N-(4-fluorophenyl)-2,6-diazaspiro[3.3]heptan-2-carbothioamide (0.100 g, 0.218 mmol) prepared in step 3 and formaldehyde (38.00% solution, 0.024 mL, 0.326 mmol) were dissolved in dichloromethane (4 mL) at room temperature, after which sodium triacetoxyborohydride (0.092 g, 0.435 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a desired compound (0.051 g, 49.5%) as a white solid form.

¹H NMR (400 MHz, CDCl₃) δ 8.03 (d, J=8.1 Hz, 2H), 7.53 (d, J=8.1 Hz, 2H), 7.05-6.79 (m, 5H), 5.54 (s, 2H), 3.77 (brs, 4H), 3.24 (s, 4H), 2.26 (s, 3H); LRMS (ES) m/z 474.6 (M⁺+1).

### Example 21: Synthesis of compound 21, N-(4-(5-(difluoromethyl)-1,3,4-oxadiazol-2-yl)benzyl)-N-(4-fluorophenyl)-6-isopropyl-2,6-diazaspiro[3.3]heptan-2-carbothioamide

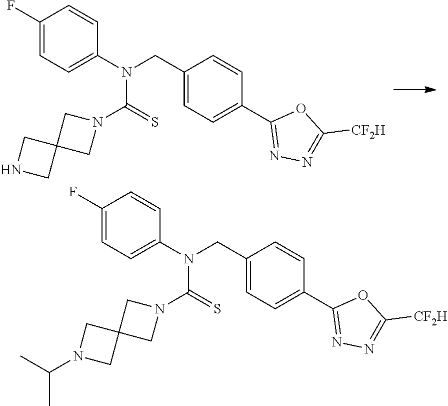

N-(4-(5-(difluoromethyl)-1,3,4-oxadiazol-2-yl)benzyl)-N-(4-fluorophenyl)-2,6-diazaspiro[3.3]heptan-2-carbothioamide (0.100 g, 0.218 mmol) prepared by the same method as described in step 3 of compound 20 and acetone (0.024 mL, 0.326 mmol) were dissolved in dichloromethane (4 mL) at room temperature, after which sodium triacetoxyborohydride (0.092 g, 0.435 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a desired compound (0.037, 33.9%) as a white solid form.

¹H NMR (400 MHz, CDCl₃) δ 8.03 (d, J=8.1 Hz, 2H), 7.53 (d, J=8.2 Hz, 2H), 7.05-6.79 (m, 5H), 5.54 (s, 2H), 3.85 (brs, 4H), 3.33 (brs, 4H), 2.48-2.47 (m, 1H), 0.95-0.89 (m, 6H); LRMS (ES) m/z 502.7 (M⁺+1).

### Example 22: Synthesis of compound 22, N-(4-(5-(difluoromethyl)-1,3,4-oxadiazol-2-yl)benzyl)-N-(4-fluorophenyl)-6-(oxetan-3-yl)-2,6-diazaspiro[3.3]heptan-2-carbothioamide

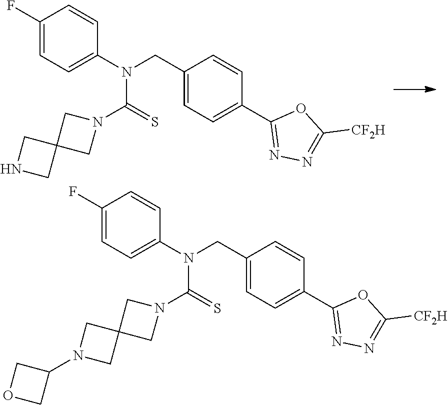

N-(4-(5-(difluoromethyl)-1,3,4-oxadiazol-2-yl)benzyl)-N-(4-fluorophenyl)-2,6-diazaspiro[3.3]heptan-2-carbothioamide (0.100 g, 0.218 mmol) prepared by the same method as described in step 3 of compound 20 and 3-oxetanone (0.021 mL, 0.326 mmol) were dissolved in dichloromethane (4 mL) at room temperature, after which sodium triacetoxyborohydride (0.092 g, 0.435 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 2.5%) and concentrated to obtain a desired compound (0.069 g, 61-5%) as a white solid form.

¹H NMR (400 MHz, CDCl₃) δ 8.03 (d, J=8.o Hz, 21H), 7.53 (d, J=8.1 Hz, 2H), 7.05-6.79 (m, 5H), 5.55 (s, 2H), 4.68 (t, J=6.7 Hz, 2H), 4.42 (t, J=5.9 Hz, 2H), 3.85-3.72 (m, 5H), 3.38 (s, 4H); LRMS (ES) m/z 516.7 (M⁺+1).

### Example 23: Synthesis of compound 23, N-(3,4-dichlorophenyl)-N-(4-(5-(difluoromethyl)-1,3,4-oxadiazol-2-yl)-2-fluorobenzyl)-6-methyl-2,6-diazaspiro[3.3]heptan-2-carbothioamide

### [Step 1] Synthesis of tert-butyl 6-((3,4-dichlorophenyl)(4-(5-(difluoromethyl)-1,3,4-oxadiazol-2-yl)-2-fluorobenzyl)carbamothioyl)-2,6-diazaspiro[3.3]heptan-2-carboxylate

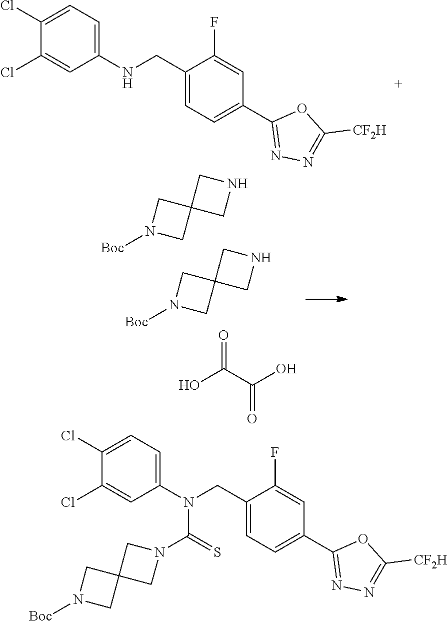

3,4-dichloro-N-(4-(5-(difluoromethyl)-1,3,4-oxadiazol-2-yl)-2-fluorobenzyl)aniline (0.930 g, 2.396 mmol), thiophosgene (0.184 mL, 2.396 mmol) and N,N-diisopropylethylamine (1.252 mL, 7.188 mmol) were dissolved in dichloromethane (20 mL), after which the resulting solution was stirred at 0° C. for 30 minutes, and then tert-butyl 2,6-diazaspiro[3.3]heptan-2-carboxylate hemioxalate (0.583 g, 1.198 mmol) was added thereinto and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 40 g cartridge; ethyl acetate/hexane=0 to 30%) and concentrated to obtain a title compound (0.280 g, 18.6%) as a yellow oil form.

### [Step 2] Synthesis of N-(3,4-dichlorophenyl)-N-(4-(5-(difluoromethyl)-1,3,4-oxadiazol-2-yl)-2-fluorobenzyl)-2,6-diazaspiro[3.3]heptan-2-carbothioamide 2,2,2-trifluoroacetate

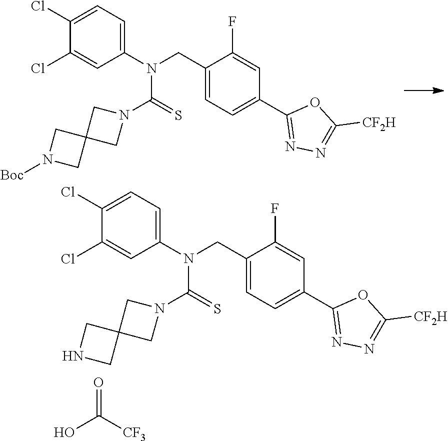

Tert-butyl 6-((3,4-dichlorophenyl)(4-(5-(difluoromethyl)-1,3,4-oxadiazol-2-yl)-2-fluorobenzyl)carbamothioyl)-2,6-diazaspiro[3.3]heptan-2-carboxylate (0.275 g, 0.438 mmol) prepared in step 1 and trifluoroacetic acid (0.335 mL, 4.376 mmol) were dissolved in dichloromethane (10 mL) at room temperature, after which the resulting solution was stirred at the same temperature for 18 hours. Solvent was removed from the reaction mixture under reduced pressure, after which a product obtained was used without a further purification process (0.275 g, 97.8%, yellow oil).

### [Step 3] Synthesis of Compound 23

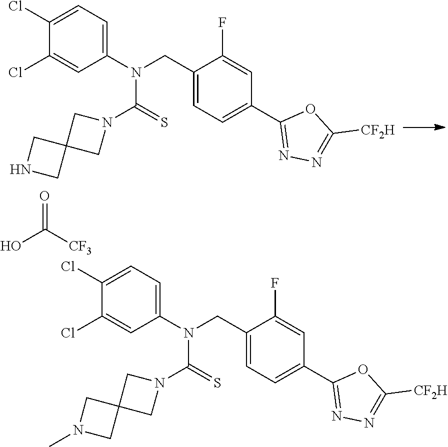

N-(3,4-dichlorophenyl)-N-(4-(5-(difluoromethyl)-1,3,4-oxadiazol-2-yl)-2-fluorobenzyl)-2,6-diazaspiro[3.3]heptan-2-carbothioamide 2,2,2-trifluoroacetate (0.150 g, 0.233 mmol) prepared in step 2, N,N-diisopropylethylamine (0.041 mL, 0.233 mmol), formaldehyde (0.014 g, 0.467 mmol) and sodium triacetoxyborohydride (0.099 g, 0.467 mmol) were dissolved in dichloromethane (10 mL) at room temperature, after which the resulting solution was stirred at the same temperature for 12 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) to obtain a title compound (0.100 g, 79.0%) as a colorless oil form.

¹H NMR (400 MHz, CDCl₃) δ 7.90˜7.87 (m, 2H), 7.72 (d, J=9.8 Hz, 1H), 7.41 (d, J=8.6 Hz, 1H), 7.26 (d, J=2.3 Hz, 1H), 7.05 (s, 0.25H), 6.98 (dd, J=8.6, 2.4 Hz, 1H), 6.92 (s, 0.5H), 6.79 (s, 0.25H), 5.55 (s, 2H), 3.87˜3.73 (m, 4H), 3.41 (s, 4H), 2.34 (s, 3H); LRMS (ES) m/z 542.2 (M⁺+1).

### Example 24: Synthesis of compound 24, N-(3,4-dichlorophenyl)-N-(4-(5-(difluoromethyl)-1,3,4-oxadiazol-2-yl)-2-fluorobenzyl)-6-(oxetan-3-yl)-2,6-diazaspiro[3.3]heptan-2-carbothioamide

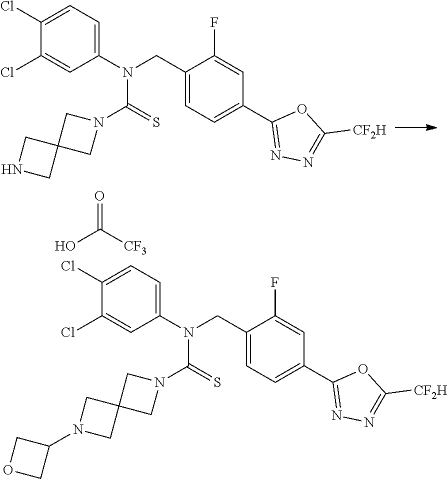

N-(3,4-dichlorophenyl)-N-(4-(5-(difluoromethyl)-1,3,4-oxadiazol-2-yl)-2-fluorobenzyl)-2,6-diazaspiro[3.3]heptan-2-carbothioamide 2,2,2-trifluoroacetate (0.150 g, 0.233 mmol) prepared by the same method as described in step 2 of compound 23, N,N-diisopropylethylamine (0.041 mL, 0.233 mmol), 3-oxetanone (0.027 mL, 0.467 mmol) and sodium triacetoxyborohydride (0.099 g, 0.467 mmol) were dissolved in dichloromethane (10 mL) at room temperature, after which the resulting solution was stirred at the same temperature for 12 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) to obtain a title compound (0.100 g, 73.3%) as a colorless oil form.

¹H NMR (400 MHz, CDCl₃) δ 7.90˜7.89 (m, 2H), 7.73 (d, J=10.0 Hz, 1H), 7.42 (d, J=8.5 Hz, 1H), 7.28˜7.27 (m, 1H), 7.05 (s, 0.25H), 6.99 (dd, J=8.5, 2.3 Hz, 1H), 6.92 (s, 0.5H), 6.79 (s, 0.25H), 5.57 (s, 2H), 4.69˜4.63 (m, 2H), 4.48˜4.45 (m, 2H), 3.94˜3.89 (m, 4H), 3.67˜3.61 (m, 1H), 3.29 (s, 4H); LRMS (ES) m/z 584.3 (M⁺+1).

### Example 25: Synthesis of compound 25, N-(3-chloro-4-fluorophenyl)-N-(4-(5-(difluoromethyl)-1,3,4-oxadiazol-2-yl)-2-fluorobenzyl)-6-methyl-2,6-diazaspiro[3.3]heptan-2-carbothioamide

### [Step 1] Synthesis of tert-butyl 6-((3-chloro-4-fluorophenyl)(4-(5-(difluoromethyl)-1,3,4-oxadiazol-2-yl)-2-fluorobenzyl)carbamothioyl)-2,6-diazaspiro[3.3]heptan-2-carboxylate

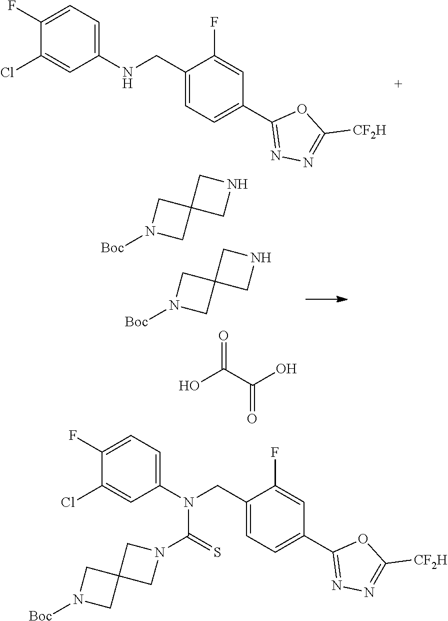

3-chloro-N-(4-(5-(difluoromethyl)-1,3,4-oxadiazol-2-yl)-2-fluorobenzyl)-4-fluoroaniline (1.000 g, 2.690 mmol), thiophosgene (0.206 mL, 2.690 mmol) and N,N-diisopropylethylamine (1.406 mL, 8.071 mmol) were dissolved in dichloromethane (20 mL), after which the resulting solution was stirred at 0° C. for 30 minutes and then tert-butyl 2,6-diazaspiro[3.3]heptan-2-carboxylate hemioxalate (0.654 g, 1.345 mmol) was added thereinto and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 40 g cartridge; ethyl acetate/hexane=0 to 30%) and concentrated to obtain a title compound (0.650 g, 39.5%) as a yellow oil form.

### [Step 2] Synthesis of N-(3-chloro-4-fluorophenyl)-N-(4-(5-(difluoromethyl)-1,3,4-oxadiazol-2-yl)-2-fluorobenzyl)-2,6-diazaspiro[3.3]heptan-2-carbothioamide 2,2,2-trifluoroacetate

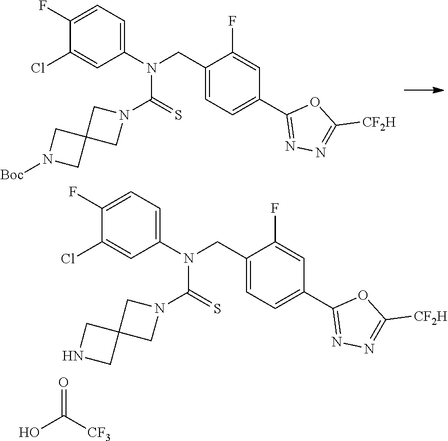

Tert-butyl 6-((3-chloro-4-fluorophenyl)(4-(5-(difluoromethyl)-1,3,4-oxadiazol-2-yl)-2-fluorobenzyl)carbamothioyl)-2,6-diazaspiro[3.3]heptan-2-carboxylate (0.680 g, 1.111 mmol) prepared in step 1 and trifluoroacetic acid (0.851 mL, 11.110 mmol) were dissolved in dichloromethane (10 mL) at room temperature, after which the resulting solution was stirred at the same temperature for 18 hours. Solvent was removed from the reaction mixture under reduced pressure, after which a product obtained was used without a further purification process (0.680 g, 97.8%, yellow oil).

### [Step 3] Synthesis of Compound 25

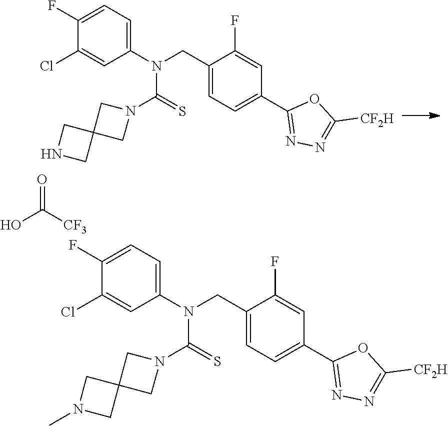

N-(3-chloro-4-fluorophenyl)-N-(4-(5-(difluoromethyl)-1,3,4-oxadiazol-2-yl)-2-fluorobenzyl)-2,6-diazaspiro[3.3]heptan-2-carbothioamide 2,2,2-trifluoroacetate (0.262 g, 0.419 mmol) prepared in step 2 and N,N-diisopropylethylamine (0.073 mL, 0.419 mmol) were dissolved in dichloromethane (10 mL), after which the resulting solution was stirred at room temperature for 30 minutes and then formaldehyde (0.025 g, 0.837 mmol) and sodium triacetoxyborohydride (0.177 g, 0.837 mmol) were added thereinto and further stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.150 g, 68.1%) as a colorless oil form.

¹H NMR (400 MHz, CDCl₃) δ 7.93˜7.88 (m, 2H), 7.72 (d, J=10.0 Hz, 1H), 7.22 (dd, J=6.3, 2.5 Hz, 1H), 7.12 (t, J=8.5 Hz, 1H), 7.05 (s, 0.25H), 7.01˜6.97 (m, 1H), 6.92 (s, 0.5H), 6.79 (s, 0.25H), 5.55 (s, 2H), 3.92 (s, 4H), 3.39 (S, 4H), 2.32 (s, 3H); LRMS (ES) m/z 526.6 (M⁺+1).

### Example 26: Synthesis of compound 26, N-(3-chloro-4-fluorophenyl)-N-(4-(5-(difluoromethyl)-1,3,4-oxadiazol-2-yl)benzyl)-6-methyl-2,6-diazaspiro[3.3]heptan-2-carbothioamide

### [Step 1] Synthesis of tert-butyl 6-((3-chloro-4-fluorophenyl)(4-(5-(difluoromethyl)-1,3,4-oxadiazol-2-yl)benzyl)carbamothioyl)-2,6-diazaspiro[3.3]heptan-2-carboxylate

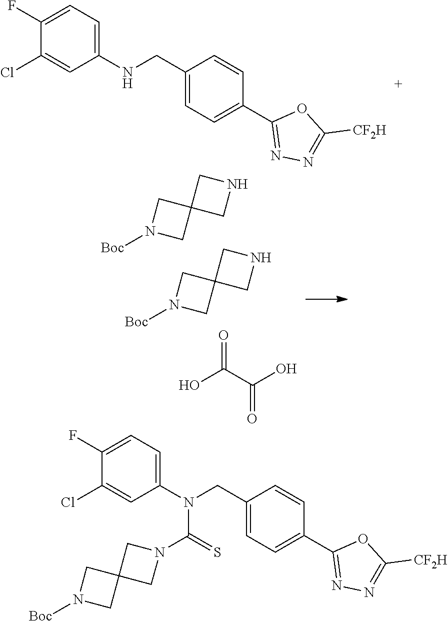

3-chloro-N-(4-(5-(difluoromethyl)-1,3,4-oxadiazol-2-yl)benzyl)-4-fluoroaniline (0.950 g, 2.686 mmol), thiophosgene (0.206 mL, 2.686 mmol) and N,N-diisopropylethylamine (1.403 mL, 8.057 mmol) were dissolved in dichloromethane (20 mL), after which the resulting solution was stirred at 0° C. for 30 minutes and then tert-butyl 2,6-diazaspiro[3.3]heptan-2-carboxylate hemioxalate (0.653 g, 1.343 mmol) was added thereinto and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 40 g cartridge; ethyl acetate/hexane=0 to 30%) and concentrated to obtain a title compound (0.853 g, 53.5%) as a yellow oil form.

### [Step 2] Synthesis of N-(3-chloro-4-fluorophenyl)-N-(4-(5-(difluoromethyl)-1,3,4-oxadiazol-2-yl)benzyl)-2,6-diazaspiro[3.3]heptan-2-carbothioamide 2,2,2-trifluoroacetate

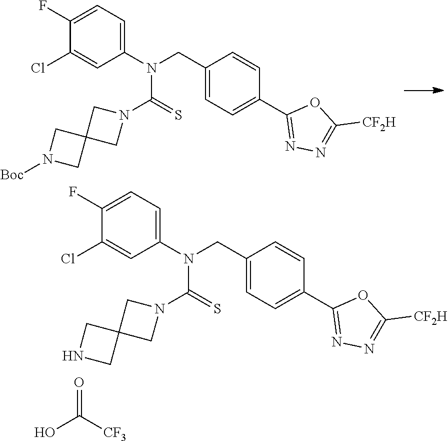

Tert-butyl 6-((3-chloro-4-fluorophenyl)(4-(5-(difluoromethyl)-1,3,4-oxadiazol-2-yl)benzyl)carbamothioyl)-2,6-diazaspiro[3.3]heptan-2-carboxylate (0.853 g, 1.436 mmol) prepared in step 1 and trifluoroacetic acid (1.100 mL, 14.359 mmol) were dissolved in dichloromethane (10 mL) at room temperature, after which the resulting solution was stirred at the same temperature for 18 hours. Solvent was removed from the reaction mixture under reduced pressure, after which a product obtained was used without a further purification process (0.853 g, 97.7%, yellow oil).

### [Step 3] Synthesis of Compound 26

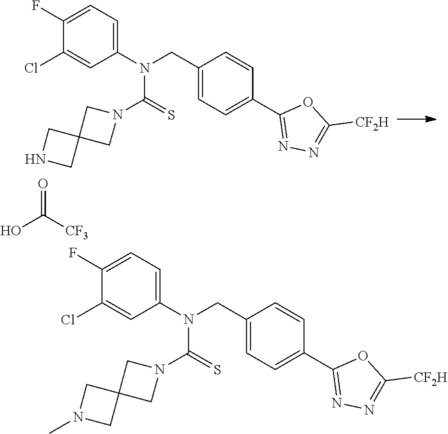

N-(3-chloro-4-fluorophenyl)-N-(4-(5-(difluoromethyl)-1,3,4-oxadiazol-2-yl)benzyl)-2,6-diazaspiro[3.3]heptan-2-carbothioamide 2,2,2-trifluoroacetate prepared in step 2 and N,N-diisopropylethylamine (0.097 mL, 0.554 mmol) were dissolved in dichloromethane (10 mL), after which the resulting solution was stirred at room temperature for 30 minutes and then formaldehyde (0.033 g, 1.109 mmol) and sodium triacetoxyborohydride (0.235 g, 1.109 mmol) were added thereinto and further stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.220 g, 78.1%) as a colorless oil form.

¹H NMR (400 MHz, CDCl₃) δ 8.04 (d, J=8.1 Hz, 2H), 7.51 (d, J=8.1 Hz, 2H), 7.16 (dd, J=6.3, 2.5 Hz, 1H), 7.10 (t, J=8.5 Hz, 1H), 7.05 (s, 0.25H), 6.95-6.91 (m, 1H), 6.92 (s, 0.5H), 6.79 (s, 0.25H), 5.50 (s, 2H), 3.86˜3.73 (m, 4H), 3.51 (s, 4H), 2.40 (s, 3H); LRMS (ES) m/z 508.5 (M⁺+1).

### Example 27: Synthesis of Compound 27, N-(3-chloro-4-fluorophenyl)-N-(4-(5-(difluoromethyl)-1,3,4-oxadiazol-2-yl)benzyl)-6-(oxetan-3-yl)-2,6-diazaspiro[3.3]heptan-2-carbothioamide

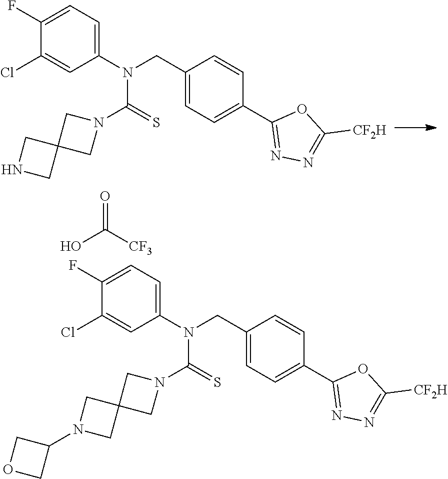

N-(3-chloro-4-fluorophenyl)-N-(4-(5-(difluoromethyl)-1,3,4-oxadiazol-2-yl)benzyl)-2,6-diazaspiro[3.3]heptan-2-carbothioamide 2,2,2-trifluoroacetate (0.320 g, 0.526 mmol) prepared by the same method as described in step 2 of compound 26 and N,N-diisopropylethylamine (0.092 mL, 0.526 mmol) were dissolved in dichloromethane (10 mL), after which the resulting solution was stirred at room temperature for 30 minutes and then 3-oxetanone (0.062 mL, 1.053 mmol) and sodium triacetoxyborohydride (0.223 g, 1.053 mmol) were added thereinto and further stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.188 g, 70.3%) as a colorless oil form.

¹H NMR (400 MHz, CDCl₃) δ 8.05 (d, J=8.1 Hz, 2H), 7.53 (d, J=8.1 Hz, 2H), 7.19 (dd, J=6.3, 2.4 Hz, 1H), 7.10 (t, J=8.5 Hz, 1H), 7.05 (s, 0.25H), 6.96-6.92 (m, 1H), 6.92 (s, 0.5H), 6.79 (s, 0.25H), 5.52 (s, 2H), 4.65 (t, J=6.6 Hz, 2H), 4.40 (t, J=5.8 Hz, 2H), 3.86˜3.75 (m, 4H), 3.67˜3.61 (m, 1H), 3.29 (s, 4H); LRMS (ES) m/z 550.4 (M⁺+1).

### Example 28: Synthesis of Compound 28, N-(3-chloro-4-fluorophenyl)-N-(4-(5-(difluoromethyl)-1,3,4-oxadiazol-2-yl)-2-fluorobenzyl)-6-isopropyl-2,6-diazaspiro[3.3]heptan-2-carbothioamide

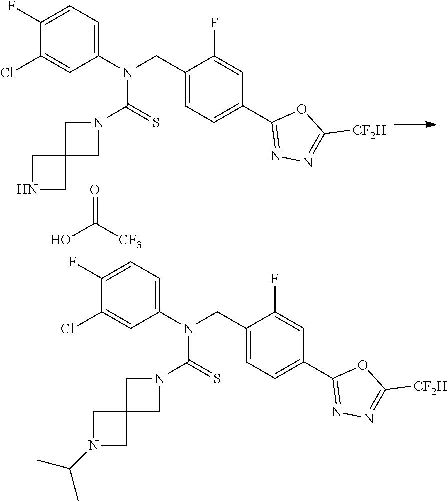

N-(3-chloro-4-fluorophenyl)-N-(4-(5-(difluoromethyl)-1,3,4-oxadiazol-2-yl)-2-fluorobenzyl)-2,6-diazaspiro[3.3]heptan-2-carbothioamide 2,2,2-trifluoroacetate (0.254 g, 0.406 mmol) prepared by the same method as described in step 2 of compound and N,N-diisopropylethylamine (0.071 mL, 0.406 mmol) were dissolved in dichloromethane (10 mL), after which the resulting solution was stirred at room temperature for 30 minutes and then acetone (0.024 g, 0.812 mmol) and sodium triacetoxyborohydride (0.172 g, 0.812 mmol) were added thereinto and further stirred at the same temperature for 18 hours. Water was poured into the reaction mixture and an extraction was performed with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.160 g, 71.2%) as a colorless oil form.

¹H NMR (400 MHz, CDCl₃) δ 7.93˜7.88 (m, 2H), 7.72 (d, J=10.0 Hz, 1H), 7.22 (dd, J=6.3, 2.4 Hz, 1H), 7.14˜7.09 (m, 1H), 7.05 (s, 0.25H), 7.01˜6.97 (m, 1H), 6.92 (s, 0.5H), 6.79 (s, 0.25H), 5.50 (s, 2H), 3.95˜3.84 (m, 4H), 3.42 (s, 4H), 2.49˜2.42 (m, 1H), 0.98˜0.96 (m, 6H); LRMS (ES) m/z 554.7 (M⁺+1).

### Example 29: Synthesis of compound 29, N-(3-chloro-4-fluorophenyl)-N-(4-(5-(difluoromethyl)-1,3,4-oxadiazol-2-yl)benzyl)-6-isopropyl-2,6-diazaspiro[3.3]heptan-2-carbothioamide

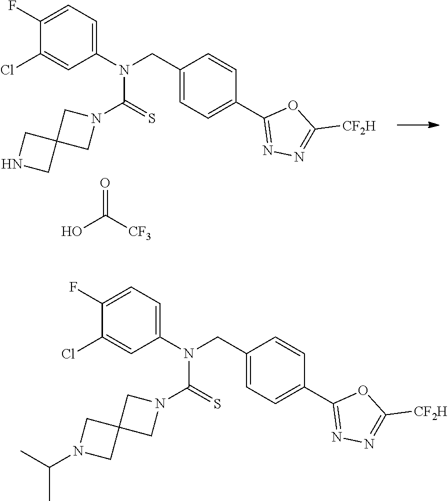

N-(3-chloro-4-fluorophenyl)-N-(4-(5-(difluoromethyl)-1,3,4-oxadiazol-2-yl)benzyl)-2,6-diazaspiro[3.3]heptan-2-carbothioamide 2,2,2-trifluoroacetate (0.325 g, 0.535 mmol) prepared by the same method as described in step 2 of compound 26 and N,N-diisopropylethylamine (0.093 mL, 0.535 mmol) were dissolved in dichloromethane (10 mL), after which the resulting solution was stirred at room temperature for 30 minutes and then acetone (0.032 g, 1.069 mmol) and sodium triacetoxyborohydride (0.227 g, 1.069 mmol) were added thereinto and further stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.199 g, 69.4%) as a colorless oil form.

¹H NMR (400 MHz, CDCl₃) δ 8.04 (d, J=8.1 Hz, 2H), 7.53 (d, J=8.1 Hz, 2H), 7.17 (dd, J=6.2, 2.2 Hz, 1H), 7.08 (t, J=8.7 Hz, 1H), 7.05 (s, 0.25H), 6.94˜6.92 (m, 1H), 6.92 (s, 0.5H), 6.79 (s, 0.25H), 5.52 (s, 2H), 3.91˜3.74 (m, 4H), 3.18 (s, 4H), 2.20˜2.16 (m, 1H), 0.88 (d, J=6.2 Hz, 6H); LRMS (ES) m/z 536.4 (M⁺+1).

### Example 30: Synthesis of compound 30, N-(4-(5-(difluoromethyl)-1,3,4-oxadiazol-2-yl)benzyl)-N-(3,4-difluorophenyl)-6-methyl-2,6-diazaspiro[3.3]heptan-2-carbothioamide

### [Step 1] Synthesis of tert-butyl 6-((4-(5-(difluoromethyl)-1,3,4-oxadiazol-2-yl)benzyl)(3,4-difluorophenyl)carbamothioyl)-2,6-diazaspiro[3.3]heptan-2-carboxylate

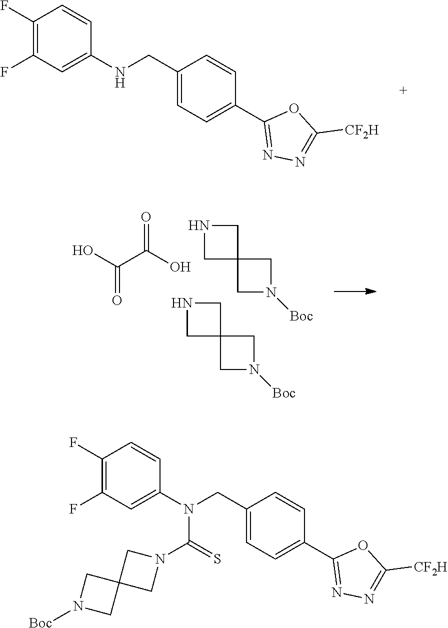

N-(4-(5-(difluoromethyl)-1,3,4-oxadiazol-2-yl)benzyl)-3,4-difluoroaniline (1.000 g, 2.965 mmol) prepared in step 1 and N,N-diisopropylethylamine (1.549 mL, 8.895 mmol) were dissolved in dichloromethane (50 mL) at 0° C., after which thiophosgene (0.341 g, 2.965 mmol) was added into the resulting solution and stirred at the same temperature. Tert-butyl 2,6-diazaspiro[3.3]heptan-2-carboxylate hemioxalate (0.866 g, 1.779 mmol) was added into the reaction mixture and further stirred at room temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; ethyl acetate/hexane=10 to 40%) and concentrated to obtain a desired compound (1.080 g, 63.1%) as a yellow solid form.

### [Step 2] Synthesis of N-(4-(5-(difluoromethyl)-1,3,4-oxadiazol-2-yl)benzyl)-N-(3,4-difluorophenyl)-2,6-diazaspiro[3.3]heptan-2-carbothioamide

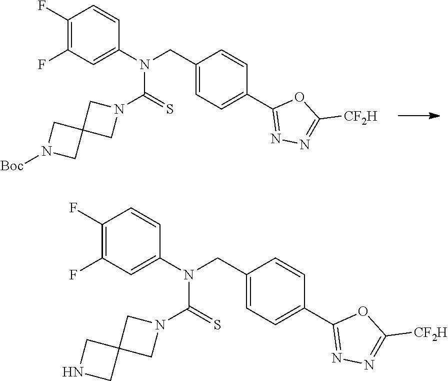 [SMILES: CC(C)(C)OC(=O)N1CC2(C1)CN(C(=S)N(Cc1ccc(-c3nnc(C(F)F)o3)cc1)c1ccc(F)c(F)c1)C2 ; Fc1ccc(N(Cc2ccc(-c3nnc(C(F)F)o3)cc2)C(=S)N2CC3(CNC3)C2)cc1F]

Tert-butyl 6-((4-(5-(difluoromethyl)-1,3,4-oxadiazol-2-yl)benzyl)(3,4-difluorophenyl)carbamothioyl)-2,6-diazaspiro[3.3]heptan-2-carboxylate (1.080 g, 1.870 mmol) prepared in step 1 and trifluoroacetic acid (1.002 mL, 13.089 mmol) were dissolved in dichloromethane (10 mL) at room temperature, after which the resulting solution was stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. An obtained product was used without a further purification process (0.864 g, 96.8%, light yellow solid).

### [Step 3] Synthesis of Compound 30

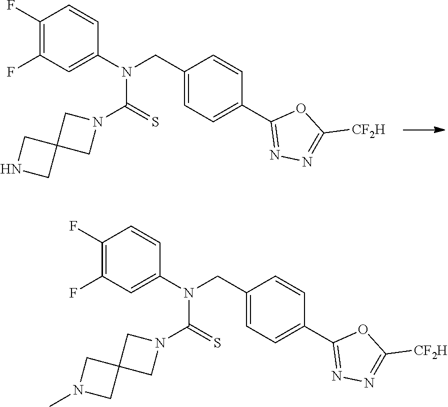 [SMILES: Fc1ccc(N(Cc2ccc(-c3nnc(C(F)F)o3)cc2)C(=S)N2CC3(CNC3)C2)cc1F ; CN1CC2(C1)CN(C(=S)N(Cc1ccc(-c3nnc(C(F)F)o3)cc1)c1ccc(F)c(F)c1)C2]

N-(4-(5-(difluoromethyl)-1,3,4-oxadiazol-2-yl)benzyl)-N-(3,4-difluorophenyl)-2,6-diazaspiro[3.3]heptan-2-carbothioamide (0.100 g, 0.209 mmol) prepared in step 2 and formaldehyde (38.00% solution in water, 0.023 mL, 0.314 mmol) were dissolved in dichloromethane (4 mL) at room temperature, after which sodium triacetoxyborohydride (0.089 g, 0.419 mmol) was added to the resulting solution and stirred at the same temperature for 18 hours. Saturated aqueous sodium hydrogen carbonate solution was poured into the reaction mixture, after which an extraction was performed with dichloromethane, then filtered via a plastic filter to remove a solid residue and an aqueous solution layer therefrom, and then concentrated under reduced pressure. The resulting concentrate was purified via column chromatography (SiO₂, 4 g cartridge; methanol/dichloromethane=0 to 5%) and concentrated to obtain a desired compound (0.030 g, 29.1%) as a white solid form.

¹H NMR (400 MHz, CDCl₃) δ 8.02 (d, J=8.1 Hz, 2H), 7.51 (d, J=8.o Hz, 2H), 7.08 (q, J=9.3 Hz, 1H), 7.01-6.78 (m, 3H), 5.51 (s, 2H), 3.82 (brs, 4H), 3.21 (s, 4H), 2.23 (s, 3H); LRMS (ES) m/z 492.7 (M⁺+1)

### Example 31: Synthesis of compound 31, N-(4-(5-(difluoromethyl)-1,3,4-oxadiazol-2-yl)benzyl)-N-(3,4-difluorophenyl)-6-isopropyl-2,6-diazaspiro[3.3]heptan-2-carbothioamide

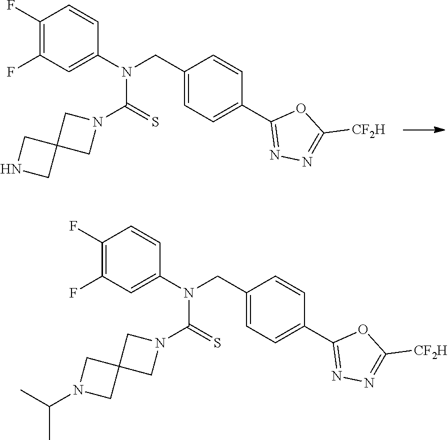 [SMILES: Fc1ccc(N(Cc2ccc(-c3nnc(C(F)F)o3)cc2)C(=S)N2CC3(CNC3)C2)cc1F ; CC(C)N1CC2(CN(C(=S)N(Cc3ccc(-c4nnc(C(F)F)o4)cc3)c3ccc(F)c(F)c3)C2)C1]

N-(4-(5-(difluoromethyl)-1,3,4-oxadiazol-2-yl)benzyl)-N-(3,4-difluorophenyl)-2,6-diazaspiro[3.3]heptan-2-carbothioamide (1.000 g, 2.094 mmol) prepared by the same method as described in step 2 of compound 30 and acetone (0.234 mL, 3.141 mmol) were dissolved in dichloromethane (4 mL) at room temperature, after which sodium triacetoxyborohydride (0.888 g, 4.189 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; methanol/dichloromethane=0 to 5%) and concentrated to obtain a desired compound (0.029 g, 2.7%) as a white solid form.

¹H NMR (400 MHz, CDCl₃) δ 8.04 (d, J=8.4 Hz, 2H), 7.53 (d, J=8.4 Hz, 2H), 7.10 (q, J=9.0 Hz, 1H), 7.05-6.80 (m, 3H), 5.52 (s, 2H), 3.84 (brs, 4H), 3.18 (s, 4H), 2.20-2.15 (m, 1H), 0.89 (d, J=6.9 Hz, 6H); LRMS (ES) m/z 520.8 (M⁺+1).

### Example 32: Synthesis of compound 32, N-(4-(5-(difluoromethyl)-1,3,4-oxadiazol-2-yl)benzyl)-N-(3,4-difluorophenyl)-6-(oxetan-3-yl)-2,6-diazaspiro[3.3]heptan-2-carbothioamide

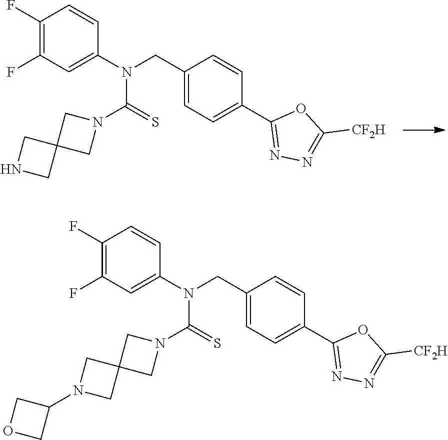 [SMILES: Fc1ccc(N(Cc2ccc(-c3nnc(C(F)F)o3)cc2)C(=S)N2CC3(CNC3)C2)cc1F ; Fc1ccc(N(Cc2ccc(-c3nnc(C(F)F)o3)cc2)C(=S)N2CC3(C2)CN(C2COC2)C3)cc1F]

N-(4-(5-(difluoromethyl)-1,3,4-oxadiazol-2-yl)benzyl)-N-(3,4-difluorophenyl)-2,6-diazaspiro[3.3]heptan-2-carbothioamide (0.100 g, 0.209 mmol) prepared by the same method as described in step 2 of compound 30 and 3-oxetanone (0.020 mL, 0.314 mmol) were dissolved in dichloromethane (4 mL) at room temperature, after which sodium triacetoxyborohydride (0.089 g, 0.419 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; methanol/dichloromethane=0 to 2.5%) and concentrated to obtain a desired compound (0.034 g, 30.4%) as a white solid form.

¹H NMR (400 MHz, CDCl₃) δ 8.05 (d, J=8.4 Hz, 2H), 7.53 (d, J=8.4 Hz, 2H), 7.14 (q, J=9.0 Hz, 1H), 7.06-6.80 (m, 3H), 5.53 (s, 2H), 4.68 (t, J=6.7 Hz, 2H), 3.89-3.70 (m, 5H), 3.38 (s, 4H); LRMS (ES) m/z 534.6 (M⁺+1).

### Example 33: Synthesis of compound 33, 6-acetyl-N-(4-(5-(difluoromethyl)-1,3,4-oxadiazol-2-yl)-2-fluorobenzyl)-N-(3,4-difluorophenyl)-2,6-diazaspiro[3.3]heptan-2-carbothioamide

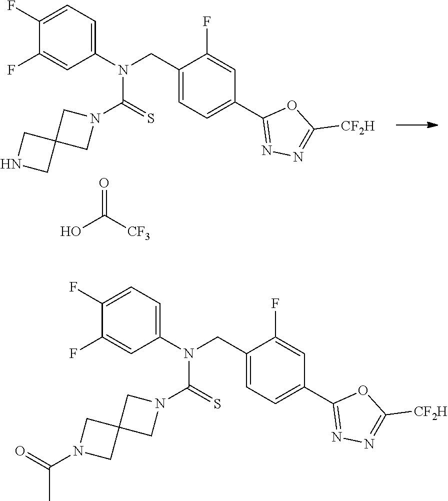

N-(4-(5-(difluoromethyl)-1,3,4-oxadiazol-2-yl)-2-fluorobenzyl)-N-(3,4-difluorophenyl)-2,6-diazaspiro[3.3]heptan-2-carbothioamide 2,2,2-trifluoroacetate (0.159 g, 0.261 mmol) prepared by the same method as described in step 3 of compound 13, N,N-diisopropylethylamine (0.091 mL, 0.522 mmol) and acetyl chloride (0.028 mL, 0.391 mmol) were dissolved in dichloromethane (20 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 70%) and concentrated to obtain a title compound (0.100 g, 71.3%) as a colorless oil form.

¹H NMR (400 MHz, CDCl₃) δ 7.92˜7.90 (m, 2H), 7.73˜7.71 (m, 1H), 7.20˜7.10 (m, 1H), 7.05 (s, 0.25H), 7.03˜6.98 (m, 1H), 6.92 (s, 0.5H), 6.92˜6.89 (m, 1H), 6.79 (s, 0.25H), 5.57 (s, 2H), 4.16˜3.80 (m, 8H), 1.82 (s, 3H); LRMS (ES) m/z 538.5 (M⁺+1).

### Example 34: Synthesis of Compound 34, N-(4-(5-(difluoromethyl)-1,3,4-oxadiazol-2-yl)-2-fluorobenzyl)-N-(3,4-difluorophenyl)-6-(oxetan-3-yl)-2,6-diazaspiro[3.3]heptan-2-carbothioamide

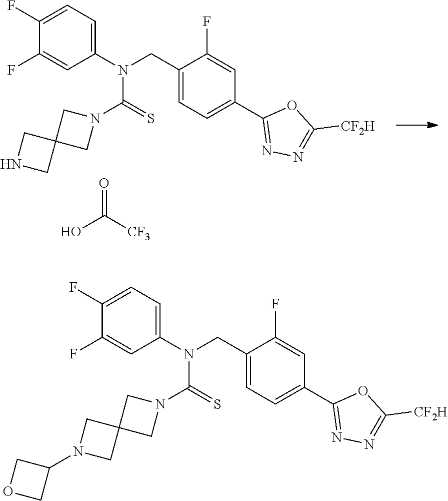

N-(4-(5-(difluoromethyl)-1,3,4-oxadiazol-2-yl)-2-fluorobenzyl)-N-(3,4-difluorophenyl)-2,6-diazaspiro[3.3]heptan-2-carbothioamide 2,2,2-trifluoroacetate (0.186 g, 0.305 mmol) prepared by the same method as described in step 3 of compound 13 and N,N-diisopropylethylamine (0.053 mL, 0.305 mmol) were dissolved in dichloromethane (10 mL), after which the resulting solution was stirred at room temperature for 30 minutes and then sodium triacetoxyborohydride (0.129 g, 0.610 mmol) and 3-oxetanone (0.044 g, 0.610 mmol) were added thereinto and further stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.100 g, 61.4%) as a colorless oil form.

¹H NMR (400 MHz, CDCl₃) δ 7.92˜7.89 (m, 2H), 7.71 (dd, J=9.9, 1.4 Hz, 1H), 7.20˜7.12 (m, 1H), 7.05 (s, 0.25H), 7.03˜6.95 (m, 1H), 6.92 (s, 0.5H), 6.89˜6.82 (m, 1H), 6.79 (s, 0.25H), 5.56 (s, 2H), 4.64 (t, J=6.7 Hz, 2H), 4.40 (dd, J=6.6, 5.2 Hz, 2H), 4.00˜3.80 (m, 4H), 3.65˜3.60 (m, 1H), 3.29 (s, 4H); LRMS (ES) m/z 552.5 (M⁺+1).

### Example 35: Synthesis of compound 35, N-(4-(5-(difluoromethyl)-1,3,4-oxadiazol-2-yl)-2-fluorobenzyl)-N-(3,4-difluorophenyl)-2-oxa-6-azaspiro[3.3]heptan-6-carbothioamide

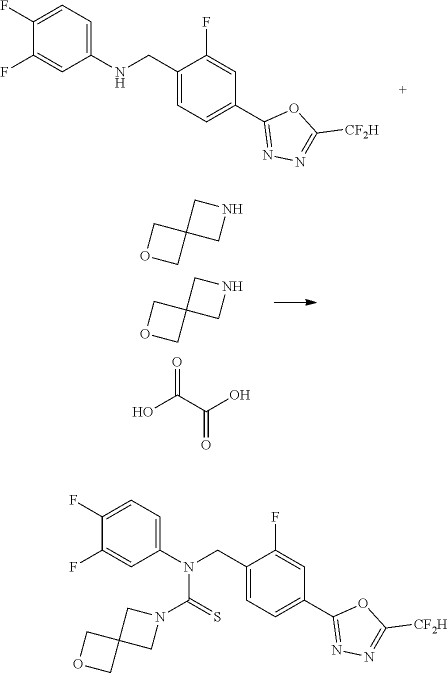

N-(4-(5-(difluoromethyl)-1,3,4-oxadiazol-2-yl)-2-fluorobenzyl)-3,4-difluoroaniline (0.330 g, 0.929 mmol) prepared by the same method as described in step 1 of compound 13, N,N-diisopropylethylamine (0.485 mL, 2.787 mmol) and thiophosgene (0.107 g, 0.929 mmol) were dissolved in dichloromethane (10 mL), after which the resulting solution was stirred at 0° C. for 30 minutes and then 2-oxa-6-azaspiro[3.3]heptan hemioxalate (0.134 g, 0.464 mmol) was added thereinto and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; ethyl acetate/hexane=0 to 70%) and concentrated to obtain a title compound (0.100 g, 21.7%) as a colorless oil form.

¹H NMR (400 MHz, CDCl₃) δ 7.94˜7.88 (m, 2H), 7.74˜7.71 (m, 1H), 7.17 (dd, J=18.2, 8.7 Hz, 1H), 7.05 (s, 0.25H), 7.02˜6.97 (m, 1H), 6.93 (s, 0.5H), 6.91˜6.87 (m, 1H), 6.80 (s, 0.25H), 5.57 (s, 2H), 4.67 (s, 4H), 3.92 (s, 4H); LRMS (ES) m/z 497.5 (M⁺+1).

### Example 36: Synthesis of compound 36, N-(4-(5-(difluoromethyl)-1,3,4-oxadiazol-2-yl)-2-fluorobenzyl)-N-(3-fluorophenyl)-6-methyl-2,6-diazaspiro[3.3]heptan-2-carbothioamide

### [Step 1] Synthesis of tert-butyl 6-((4-(5-(difluoromethyl)-1,3,4-oxadiazol-2-yl)-2-fluorobenzyl)(3-fluorophenyl)carbamothioyl)-2,6-diazaspiro[3.3]heptan-2-carboxylate

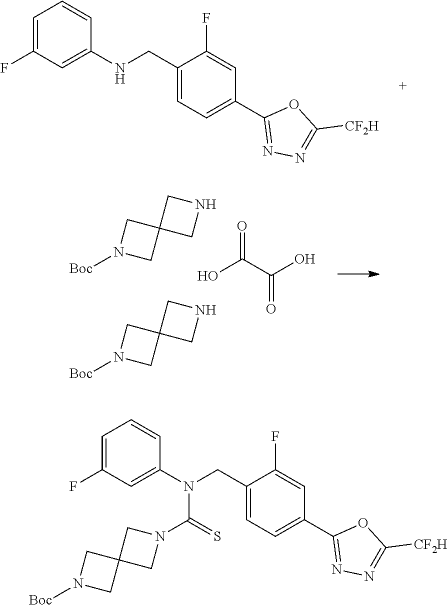

N-(4-(5-(difluoromethyl)-1,3,4-oxadiazol-2-yl)-2-fluorobenzyl)-3-fluoroaniline (1.000 g, 2.965 mmol) and N,N-diisopropylethylamine (1.033 mL, 5.930 mmol) were dissolved in dichloromethane (30 mL) at 0° C., after which thiophosgene (0.309 mL, 3.261 mmol) was added into the resulting solution and stirred at the same temperature. Tert-butyl 2,6-diazaspiro[3.3]heptan-2-carboxylate hemioxalate (0.866 g, 1.779 mmol) was added into the reaction mixture and further stirred at room temperature for 18 hours.

The reaction mixture was purified via column chromatography (SiO₂, 24 g cartridge; ethyl acetate/hexane=10 to 60%) and concentrated to obtain a desired compound (0.560 g, 32.7%) as a light yellow oil form.

### [Step 2] Synthesis of N-(4-(5-(difluoromethyl)-1,3,4-oxadiazol-2-yl)-2-fluorobenzyl)-N-(3-fluorophenyl)-2,6-diazaspiro[3.3]heptan-2-carbothioamide

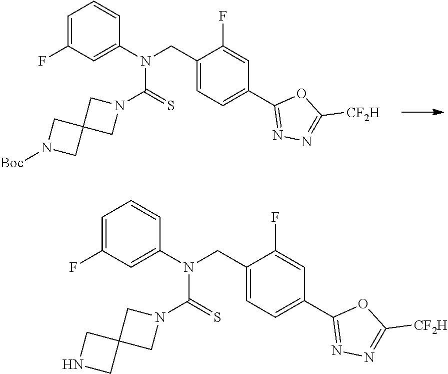 [SMILES: CC(C)(C)OC(=O)N1CC2(C1)CN(C(=S)N(Cc1ccc(-c3nnc(C(F)F)o3)cc1F)c1cccc(F)c1)C2 ; Fc1cccc(N(Cc2ccc(-c3nnc(C(F)F)o3)cc2F)C(=S)N2CC3(CNC3)C2)c1]

Tert-butyl 6-((4-(5-(difluoromethyl)-1,3,4-oxadiazol-2-yl)-2-fluorobenzyl)(3-fluorophenyl)carbamothioyl)-2,6-diazaspiro[3.3]heptan-2-carboxylate (0.560 g, 0.970 mmol) prepared in step 1 and trifluoroacetic acid (0.520 mL, 6.787 mmol) were dissolved in dichloromethane (6 mL) at room temperature, after which the resulting solution was stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. An obtained product was used without a further purification process (0.420 g, 90.7%, yellow solid).

### [Step 3] Synthesis of Compound 36

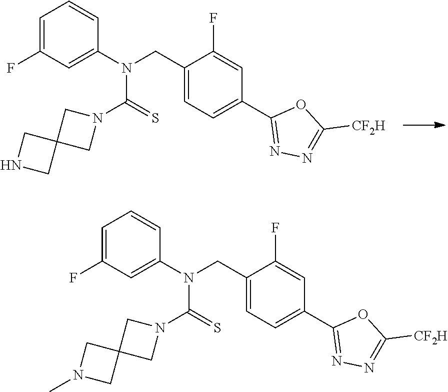 [SMILES: Fc1cccc(N(Cc2ccc(-c3nnc(C(F)F)o3)cc2F)C(=S)N2CC3(CNC3)C2)c1 ; CN1CC2(C1)CN(C(=S)N(Cc1ccc(-c3nnc(C(F)F)o3)cc1F)c1cccc(F)c1)C2]

N-(4-(5-(difluoromethyl)-1,3,4-oxadiazol-2-yl)-2-fluorobenzyl)-N-(3-fluorophenyl)-2,6-diazaspiro[3.3]heptan-2-carbothioamide (0.100 g, 0.209 mmol) prepared in step 2 and formaldehyde (38.00% solution in water, 0.023 mL, 0.314 mmol) were dissolved in dichloromethane (4 mL) at room temperature, after which sodium triacetoxyborohydride (0.089 g, 0.419 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a desired compound (0.008 g, 7.8%) as a light yellow solid form.

¹H NMR (400 MHz, CDCl₃) δ 7.94˜7.88 (m, 2H), 7.71 (d, J=10.2 Hz, 1H), 7.34˜7.29 (m, 1H), 7.05˜6.79 (m, 4H), 5.61 (s, 2H), 3.84 (brs, 4H), 3.23 (s, 4H), 2.26 (s, 3H); LRMS (ES) m/z 492.2 (M⁺+1).

### Example 37: Synthesis of compound 37, N-(4-(5-(difluoromethyl)-1,3,4-oxadiazol-2-yl)-2-fluorobenzyl)-N-(3-fluorophenyl)-6-isopropyl-2,6-diazaspiro[3.3]heptan-2-carbothioamide

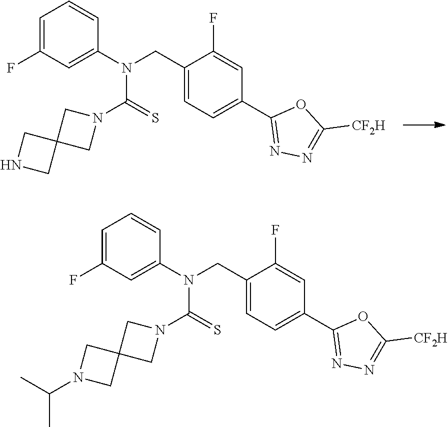 [SMILES: Fc1cccc(N(Cc2ccc(-c3nnc(C(F)F)o3)cc2F)C(=S)N2CC3(CNC3)C2)c1 ; CC(C)N1CC2(CN(C(=S)N(Cc3ccc(-c4nnc(C(F)F)o4)cc3F)c3cccc(F)c3)C2)C1]

N-(4-(5-(difluoromethyl)-1,3,4-oxadiazol-2-yl)-2-fluorobenzyl)-N-(3-fluorophenyl)-2,6-diazaspiro[3.3]heptan-2-carbothioamide (0.100 g, 0.209 mmol) prepared by the same method as described in step 2 of compound 36 and acetone (0.023 mL, 0.314 mmol) were dissolved in dichloromethane (4 mL) at room temperature, after which sodium triacetoxyborohydride (0.089 g, 0.419 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a desired compound (0.006 g, 5.5%) as a light yellow solid form.

¹H NMR (400 MHz, CDCl₃) δ 7.94˜7.87 (m, 2H), 7.71 (dd, J=9.9, 1.3 Hz, 1H), 7.33˜7.27 (m, 1H), 7.05˜6.79 (m, 4H), 5.61 (s, 2H), 3.80 (brs, 4H), 3.20 (s, 4H), 2.22˜ 2.19 (m, 1H), 0.88 (d, J=4.8 Hz, 6H); LRMS (ES) m/z 520.4 (M⁺+1).

### Example 38: Synthesis of compound 38, N-(4-(5-(difluoromethyl)-1,3,4-oxadiazol-2-yl)-2-fluorobenzyl)-N-(3-fluorophenyl)-6-(oxetan-3-yl)-2,6-diazaspiro[3.3]heptan-2-carbothioamide

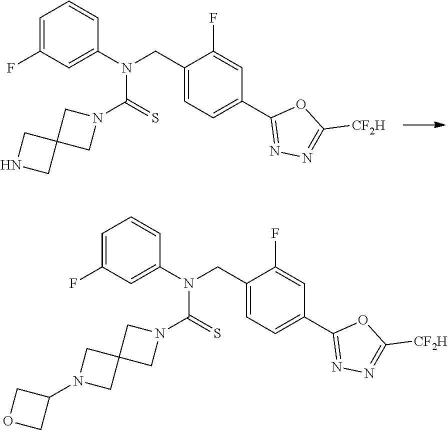 [SMILES: Fc1cccc(N(Cc2ccc(-c3nnc(C(F)F)o3)cc2F)C(=S)N2CC3(CNC3)C2)c1 ; Fc1cccc(N(Cc2ccc(-c3nnc(C(F)F)o3)cc2F)C(=S)N2CC3(C2)CN(C2COC2)C3)c1]

N-(4-(5-(difluoromethyl)-1,3,4-oxadiazol-2-yl)-2-fluorobenzyl)-N-(3-fluorophenyl)-2,6-diazaspiro[3.3]heptan-2-carbothioamide (0.100 g, 0.209 mmol) prepared by the same method as described in step 2 of compound 36 and 3-oxetanone (0.020 mL, 0.314 mmol) were dissolved in dichloromethane (4 mL) at room temperature, after which sodium triacetoxyborohydride (0.089 g, 0.419 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 2.5%) and concentrated to obtain a desired compound (0.004 g, 3.6%) as a light yellow solid form.

¹H NMR (400 MHz, CDCl₃) δ 7.94˜7.88 (m, 2H), 7.72 (dd, J=10.0, 1.3 Hz, 1H), 7.35˜7.29 (m, 1H), 7.05-6.79 (m, 4H), 4.66 (t, J=6.7 Hz, 2H), 4.42-4.41 (m, 2H), 3.88-3.67 (m, 5H), 3.32 (s, 4H); LRMS (ES) m/z 534.3 (M⁺+1).

### Example 39: Synthesis of compound 39, (1S,4S)—N-(4-(5-(difluoromethyl)-1,3,4-oxadiazol-2-yl)-2-fluorobenzyl)-N-(4-fluorophenyl)-5-methyl-2,5-diazabicyclo[2.2.1]heptan-2-carbothioamide

### [Step 1] Synthesis of tert-butyl (1S,4S)-5-((4-(5-(difluoromethyl)-1,3,4-oxadiazol-2-yl)-2-fluorobenzyl)(4-fluorophenyl)carbamothioyl)-2,5-diazabicyclo[2.2.1]heptan-2-carboxylate

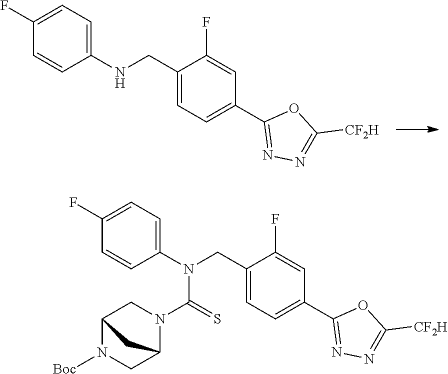 [SMILES: Fc1ccc(NCc2ccc(-c3nnc(C(F)F)o3)cc2F)cc1 ; CC(C)(C)OC(=O)N1C[C@@H]2C[C@H]1CN2C(=S)N(Cc1ccc(-c2nnc(C(F)F)o2)cc1F)c1ccc(F)cc1]

N-(4-(5-(difluoromethyl)-1,3,4-oxadiazol-2-yl)-2-fluorobenzyl)-4-fluoroaniline (1.000 g, 2.965 mmol) prepared in step 1 of compound 17 and N,N-diisopropylethylamine (1.549 mL, 8.895 mmol) were dissolved in dichloromethane (30 mL) at 0° C., after which thiophosgene (0.227 mL, 2.965 mmol) was added into the resulting solution and stirred at the same temperature. Tert-butyl (1S,4S)-2,5-diazabicyclo[2.2.1]heptan-2-carboxylate (0.705 g, 3.558 mmol) was added into the reaction mixture and further stirred at room temperature for 18 hours. Saturated aqueous sodium hydrogen carbonate solution was poured into the reaction mixture and an organic layer was extracted with dichloromethane. The organic layer was washed with saturated aqueous sodium chloride solution, dehydrated with anhydrous magnesium sulfate, filtered, and concentrated under reduced pressure. The resulting concentrate was purified via column chromatography (SiO₂, 24 g cartridge; ethyl acetate/hexane=10 to 40%) and concentrated to obtain a desired compound (1.120 g, 65.4%) as a light yellow solid form.

### [Step 2] Synthesis of (1S,4S)—N-(4-(5-(difluoromethyl)-1,3,4-oxadiazol-2-yl)-2-fluorobenzyl)-N-(4-fluorophenyl)-2,5-diazabicyclo[2.2.1]heptan-2-carbothioamide

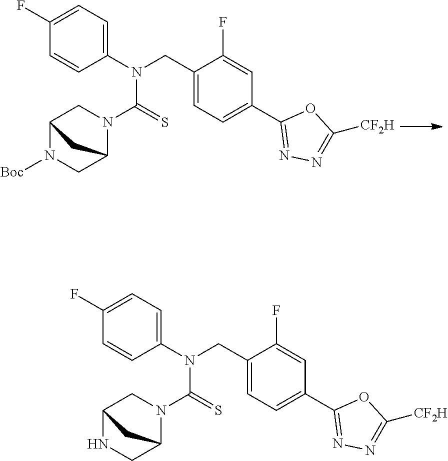 [SMILES: CC(C)(C)OC(=O)N1C[C@@H]2C[C@H]1CN2C(=S)N(Cc1ccc(-c2nnc(C(F)F)o2)cc1F)c1ccc(F)cc1 ; Fc1ccc(N(Cc2ccc(-c3nnc(C(F)F)o3)cc2F)C(=S)N2C[C@@H]3C[C@H]2CN3)cc1]

Tert-butyl (1S,4S)-5-((4-(5-(difluoromethyl)-1,3,4-oxadiazol-2-yl)-2-fluorobenzyl)(4-fluorophenyl)carbamothioyl)-2,5-diazabicyclo[2.2.1]heptan-2-carboxylate (1.120 g, 1.939 mmol) prepared in step 1 and trifluoroacetic acid (1.039 mL, 13.573 mmol) were dissolved in dichloromethane (10 mL) at room temperature, after which the resulting solution was stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. An obtained product was used without a further purification process (0.780 g, 84.2%, yellow solid).

### [Step 3] Synthesis of Compound 39

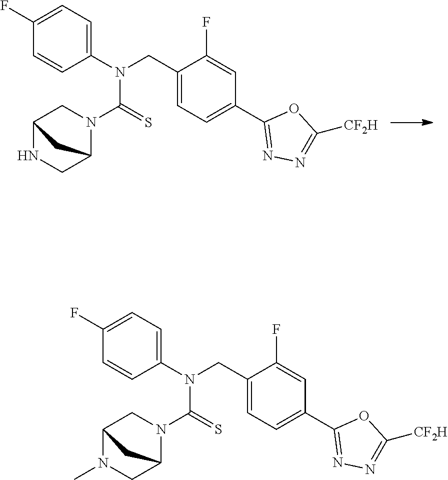 [SMILES: Fc1ccc(N(Cc2ccc(-c3nnc(C(F)F)o3)cc2F)C(=S)N2C[C@@H]3C[C@H]2CN3)cc1 ; CN1C[C@@H]2C[C@H]1CN2C(=S)N(Cc1ccc(-c2nnc(C(F)F)o2)cc1F)c1ccc(F)cc1]

(1S,4S)—N-(4-(5-(difluoromethyl)-1,3,4-oxadiazol-2-yl)-2-fluorobenzyl)-N-(4-fluorophenyl)-2,5-diazabicyclo[2.2.1]heptan-2-carbothioamide (0.150 g, 0.314 mmol) prepared in step 2 and formaldehyde (38.oo % solution in water, 0.034 mL, 0.471 mmol) were dissolved in dichloromethane (4 mL) at room temperature, after which sodium triacetoxyborohydride (0.133 g, 0.628 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a desired compound (0.070 g, 45.3%) as a white solid form.

¹H NMR (400 MHz, CDCl₃) δ 7.89˜7.82 (m, 2H), 7.75 (dd, J=10.2, 1.3 Hz, 1H), 7.13-7.08 (m, 2H), 7.13-6.79 (m, 3H), 5.64 (d, J=15.9 Hz, 1H), 5.31 (d, J=3.4 Hz, 1H), 4.94 (s, 1H), 3.35˜3.30 (m, 2H), 2.79˜2.74 (m, 3H), 2.33 (s, 3H), 1.85 (d, J=10.0 Hz, 1H), 1.57 (dd, J=10.0, 1.5 Hz, 1H); LRMS (ES) m/z 492.4 (M⁺+1).

### Example 40: Synthesis of compound 40, (1S,4S)—N-(4-(5-(difluoromethyl)-1,3,4-oxadiazol-2-yl)-2-fluorobenzyl)-N-(4-fluorophenyl)-5-isopropyl-2,5-diazabicyclo[2.2.1]heptan-2-carbothioamide

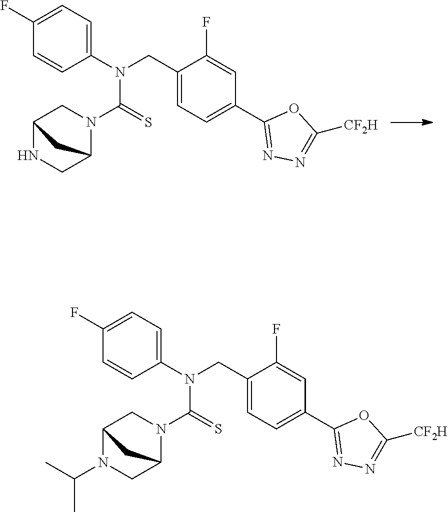 [SMILES: Fc1ccc(N(Cc2ccc(-c3nnc(C(F)F)o3)cc2F)C(=S)N2C[C@@H]3C[C@H]2CN3)cc1 ; CC(C)N1C[C@@H]2C[C@H]1CN2C(=S)N(Cc1ccc(-c2nnc(C(F)F)o2)cc1F)c1ccc(F)cc1]

(1S,4S)—N-(4-(5-(difluoromethyl)-1,3,4-oxadiazol-2-yl)-2-fluorobenzyl)-N-(4-fluorophenyl)-2,5-diazabicyclo[2.2.1]heptan-2-carbothioamide (0.150 g, 0.314 mmol) prepared by the same method as described in step 2 of compound 39 and acetone (0.035 mL, 0.471 mmol) were dissolved in dichloromethane (4 mL) at room temperature, after which sodium triacetoxyborohydride (0.133 g, 0.628 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a desired compound (0.087 g, 53.3%) as a light yellow solid form.

¹H NMR (400 MHz, CDCl₃) δ 7.89˜7.82 (m, 2H), 7.76 (d, J=9.6 Hz, 1H), 7.13˜7.09 (m, 2H), 7.06˜6.80 (m, 3H), 5.61 (d, J=15.9 Hz, 1H), 5.33 (d, J=15.8 Hz, 1H), 4.91 (s, 1H), 3.64 (s, 1H), 3.37 (s, 1H), 3.04˜3.02 (m, 1H), 2.72˜2.70 (m, 2H), 2.49 (s, 1H), 1.87 (d, J=9.1 Hz, 1H), 1.60 (d, J=10.1 Hz, 1H), 0.92˜0.88 (m, 6H); LRMS (ES) m/z 520.4 (M⁺+1).

### Example 41: Synthesis of compound 41, (1S,4S)—N-(4-(5-(difluoromethyl)-1,3,4-oxadiazol-2-yl)-2-fluorobenzyl)-N-(4-fluorophenyl)-5-(oxetan-3-yl)-2,5-diazabicyclo[2.2.1]heptan-2-carbothioamide

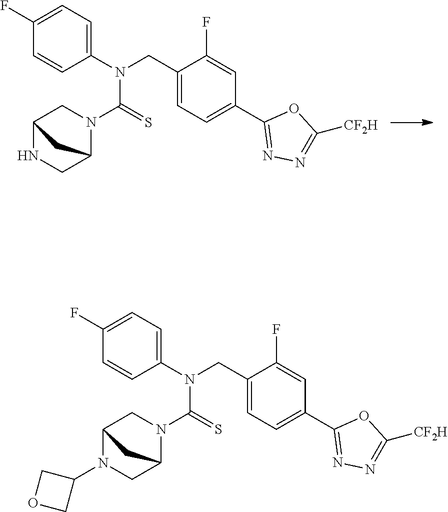 [SMILES: Fc1ccc(N(Cc2ccc(-c3nnc(C(F)F)o3)cc2F)C(=S)N2C[C@@H]3C[C@H]2CN3)cc1 ; Fc1ccc(N(Cc2ccc(-c3nnc(C(F)F)o3)cc2F)C(=S)N2C[C@@H]3C[C@H]2CN3C2COC2)cc1]

(1S,4S)—N-(4-(5-(difluoromethyl)-1,3,4-oxadiazol-2-yl)-2-fluorobenzyl)-N-(4-fluorophenyl)-2,5-diazabicyclo[2.2.1]heptan-2-carbothioamide (0.100 g, 0.209 mmol) prepared by the same method as described in step 2 of compound 39 and 3-oxetanone (0.020 mL, 0.314 mmol) were dissolved in dichloromethane (4 mL) at room temperature, after which sodium triacetoxyborohydride (0.089 g, 0.419 mmol) was added to the resulting solution and stirred at the same temperature for 18 hours. Saturated aqueous sodium hydrogen carbonate solution was poured into the reaction mixture, an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=50 to 90%) and concentrated to obtain a desired compound (0.068 g, 60.9%) as a white solid form.

¹H NMR (400 MHz, CDCl₃) δ 7.89˜7.85 (m, 2H), 7.75 (d, J=10.5 Hz, 1H), 7.12˜7.08 (m, 2H), 7.05˜6.79 (m, 3H), 5.58 (d, J=15.7 Hz, 1H), 5.34 (d, J=15.7 Hz, 1H), 4.97 (s, 1H), 4.67˜4.63 (m, 2H), 4.49˜4.44 (m, 2H), 3.87˜3.81 (m, 1H), 3.32 (s, 1H), 3.12˜3.09 (m, 2H), 2.75 (d, J=8.4 Hz, 1H), 2.70-2.69 (m, 1H), 1.80 (d, J=10.0 Hz, 1H), 1.57 (d, J=10.0 Hz, 1H); LRMS (ES) m/z 534.4 (M⁺+1).

### Example 42: Synthesis of compound 42, (1S,4S)—N-(4-(5-(difluoromethyl)-1,3,4-oxadiazol-2-yl)-2-fluorobenzyl)-N-(3,4-difluorophenyl)-5-methyl-2,5-diazabicyclo[2.2.1]heptan-2-carbothioamide

### [Step 1] Synthesis of tert-butyl (1S,4S)-5-((4-(5-(difluoromethyl)-1,3,4-oxadiazol-2-yl)-2-fluorobenzyl)(3,4-difluorophenyl)carbamothioyl)-2,5-diazabicyclo[2.2.1]heptan-2-carboxylate

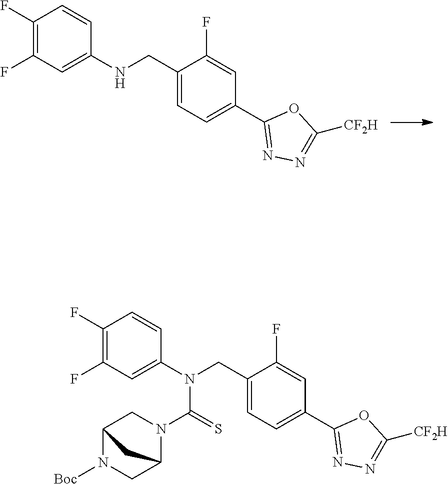 [SMILES: Fc1ccc(NCc2ccc(-c3nnc(C(F)F)o3)cc2F)cc1F ; CC(C)(C)OC(=O)N1C[C@@H]2C[C@H]1CN2C(=S)N(Cc1ccc(-c2nnc(C(F)F)o2)cc1F)c1ccc(F)c(F)c1]

N-(4-(5-(difluoromethyl)-1,3,4-oxadiazol-2-yl)-2-fluorobenzyl)-3,4-difluoroaniline (1.000 g, 2.815 mmol) prepared by the same method as described in step 1 of example 13, thiophosgene (0.216 mL, 2.815 mmol) and N,N-diisopropylethylamine (1.716 mL, 9.852 mmol) were dissolved in dichloromethane (30 mL), after which the resulting solution was stirred at 0° C. for 30 minutes and then tert-butyl (1S,4S)-2,5-diazabicyclo[2.2.1]heptan-2-carboxylate (0.558 g, 2.815 mmol) was added thereinto and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 40 g cartridge; ethyl acetate/hexane=0 to 30%) and concentrated to obtain a title compound (0.460 g, 27.4%) as a yellow oil form.

### [Step 2] Synthesis of (1S,4S)—N-(4-(5-(difluoromethyl)-1,3,4-oxadiazol-2-yl)-2-fluorobenzyl)-N-(3,4-difluorophenyl)-2,5-diazabicyclo[2.2.1]heptan-2-carbothioamide

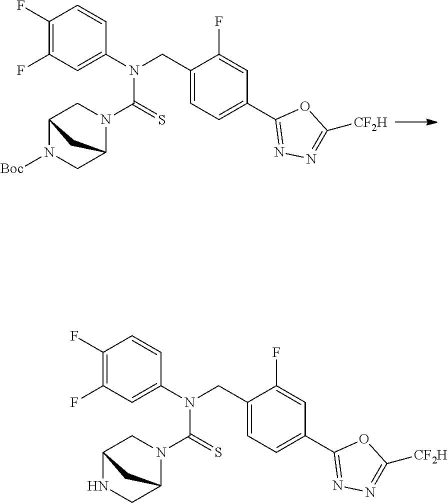 [SMILES: CC(C)(C)OC(=O)N1C[C@@H]2C[C@H]1CN2C(=S)N(Cc1ccc(-c2nnc(C(F)F)o2)cc1F)c1ccc(F)c(F)c1 ; Fc1ccc(N(Cc2ccc(-c3nnc(C(F)F)o3)cc2F)C(=S)N2C[C@@H]3C[C@H]2CN3)cc1F]

Tert-butyl (1S,4S)-5-((4-(5-(difluoromethyl)-1,3,4-oxadiazol-2-yl)-2-fluorobenzyl)(3,4-difluorophenyl)carbamothioyl)-2,5-diazabicyclo[2.2.1]heptan-2-carboxylate (0.460 g, 0.772 mmol) prepared in step 1 and trifluoroacetic acid (0.591 mL, 7.723 mmol) were dissolved in dichloromethane (10 mL) at room temperature, after which the resulting solution was stirred at the same temperature for 18 hours. Solvent was removed from the reaction mixture under reduced pressure, after which saturated aqueous sodium hydrogen carbonate solution was poured into the resulting concentrate and an organic layer was extracted with dichloromethane. The organic layer was washed with saturated aqueous sodium chloride solution, dehydrated with anhydrous sodium sulfate, filtered, and concentrated under reduced pressure. A title compound was used without a further purification process (0.350 g, 91.5%, colorless oil).

### [Step 3] Synthesis of Compound 42

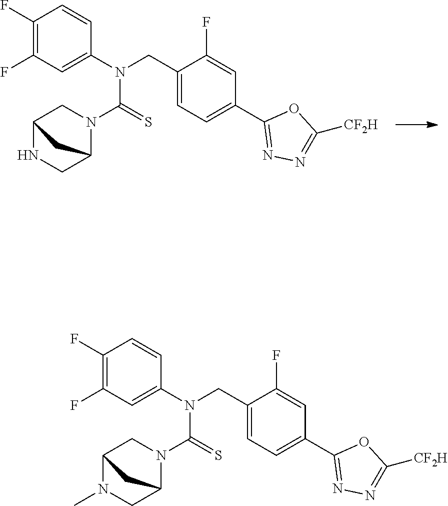 [SMILES: Fc1ccc(N(Cc2ccc(-c3nnc(C(F)F)o3)cc2F)C(=S)N2C[C@@H]3C[C@H]2CN3)cc1F ; CN1C[C@@H]2C[C@H]1CN2C(=S)N(Cc1ccc(-c2nnc(C(F)F)o2)cc1F)c1ccc(F)c(F)c1]

(1S,4S)—N-(4-(5-(difluoromethyl)-1,3,4-oxadiazol-2-yl)-2-fluorobenzyl)-N-(3,4-difluorophenyl)-2,5-diazabicyclo[2.2.1]heptan-2-carbothioamide (0.168 g, 0.339 mmol) prepared in step 2, formaldehyde (0.020 g, 0.678 mmol) and N,N-diisopropylethylamine (0.118 mL, 0.678 mmol) were dissolved in dichloromethane (20 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.110 g, 63.7%) as a colorless oil form.

¹H NMR (400 MHz, CDCl₃) δ 7.88 (dd, J=8.0, 1.6 Hz, 1H), 7.81˜7.75 (m, 2H), 7.15˜7.05 (m, 1H), 7.02 (s, 0.25H), 7.01˜6.97 (m, 1H), 6.92 (s, 0.5H), 6.91˜689.00 (m, 1H), 6.79 (s, 0.25H), 5.62 (d, J=15.9 Hz, 1H), 5.21 (d, J=16.0 Hz, 1H), 4.96 (s, 1H), 3.47˜3.45 (m, 2H), 2.88˜2.80 (m, 3H), 2.38 (s, 3H), 1.94 (d, J=10.4 Hz, 1H), 1.64 (d, J=10.2 Hz, 1H); LRMS (ES) m/z 510.8 (M⁺+1).

### Example 43: Synthesis of compound 43, (1S,4S)—N-(4-(5-(difluoromethyl)-1,3,4-oxadiazol-2-yl)-2-fluorobenzyl)-N-(3,4-difluorophenyl)-5-(oxetan-3-yl)-2,5-diazabicyclo[2.2.1]heptan-2-carbothioamide

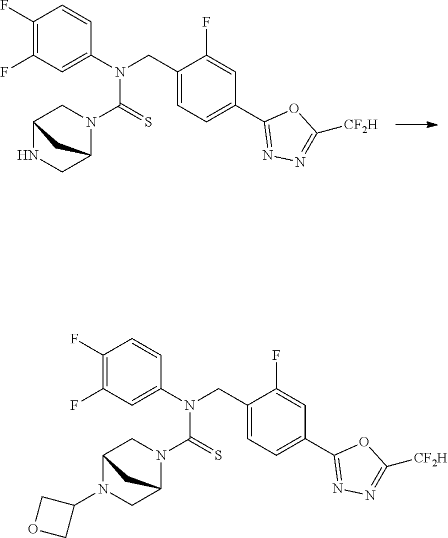 [SMILES: Fc1ccc(N(Cc2ccc(-c3nnc(C(F)F)o3)cc2F)C(=S)N2C[C@@H]3C[C@H]2CN3)cc1F ; Fc1ccc(N(Cc2ccc(-c3nnc(C(F)F)o3)cc2F)C(=S)N2C[C@@H]3C[C@H]2CN3C2COC2)cc1F]

(1S,4S)—N-(4-(5-(difluoromethyl)-1,3,4-oxadiazol-2-yl)-2-fluorobenzyl)-N-(3,4-difluorophenyl)-2,5-diazabicyclo[2.2.1]heptan-2-carbothioamide (0.126 g, 0.254 mmol) prepared by the same method as described in step 2 of compound 42, 3-oxetanone (0.030 mL, 0.509 mmol) and N,N-diisopropylethylamine (0.089 mL, 0.509 mmol) were dissolved in dichloromethane (20 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.088 g, 62.7%) as a colorless oil form.

¹H NMR (400 MHz, CDCl₃) δ 7.89˜7.76 (m, 3H), 7.15˜7.05 (m, 1H), 7.02 (s, 0.25H), 7.00˜6.97 (m, 1H), 6.92 (s, 0.5H), 6.91˜6.87 (m, 1H), 6.79 (s, 0.25H), 5.53 (d, J=15.8 Hz, 1H), 5.29 (d, J=15.8 Hz, 1H), 4.96 (s, 1H), 4.65 (dd, J=13.8, 6.7 Hz, 2H), 4.48˜4.41 (m, 2H), 3.84˜3.81 (m, 1H), 3.81 (s, 1H), 3.25˜3.00 (m, 2H), 2.78˜2.75 (m, 2H), 1.82 (d, J=10.1 Hz, 1H), 1.61 (d, J=27.1 Hz, 1H); LRMS (ES) m/z 552.8 (M⁺+1).

### Example 44: Synthesis of Compound 44, N-(4-(5-(difluoromethyl)-1,3,4-oxadiazol-2-yl)-2-fluorobenzyl)-N-(3,4-difluorophenyl)-2-methyl-2,7-diazaspiro[3.5]nonan-7-carbothioamide

### [Step 1] Synthesis of tert-butyl 7-((4-(5-(difluoromethyl)-1,3,4-oxadiazol-2-yl)-2-fluorobenzyl)(3,4-difluorophenyl)carbamothioyl)-2,7-diazaspiro[3.5]nonan-2-carboxylate

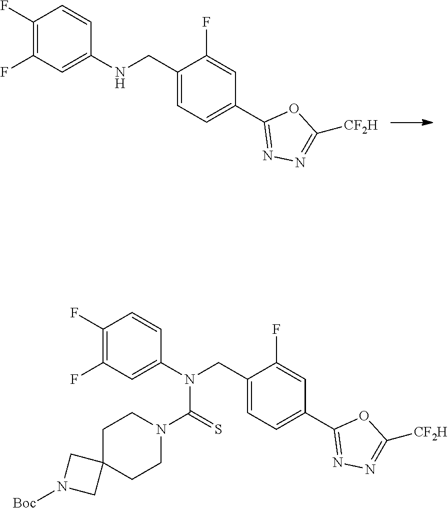 [SMILES: Fc1ccc(NCc2ccc(-c3nnc(C(F)F)o3)cc2F)cc1F ; CC(C)(C)OC(=O)N1CC2(CCN(C(=S)N(Cc3ccc(-c4nnc(C(F)F)o4)cc3F)c3ccc(F)c(F)c3)CC2)C1]

N-(4-(5-(difluoromethyl)-1,3,4-oxadiazol-2-yl)-2-fluorobenzyl)-3,4-difluoroaniline (1.000 g, 2.815 mmol) prepared by the same method as described in step 1 of compound 13, thiophosgene (0.216 mL, 2.815 mmol) and N,N-diisopropylethylamine (1.716 mL, 9.852 mmol) were dissolved in dichloromethane (30 mL), after which the resulting solution was stirred at ° C. for 30 minutes and then tert-butyl 2,7-diazaspiro[3.5]nonan-2-carboxylate (0.637 g, 2.815 mmol) was added thereinto and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 40 g cartridge; ethyl acetate/hexane=0 to 30%) and concentrated to obtain a title compound (0.600 g, 34.2%) as a yellow oil form.

### [Step 2] Synthesis of N-(4-(5-(difluoromethyl)-1,3,4-oxadiazol-2-yl)-2-fluorobenzyl)-N-(3,4-difluorophenyl)-2,7-diazaspiro[3.5]nonan-7-carbothioamide

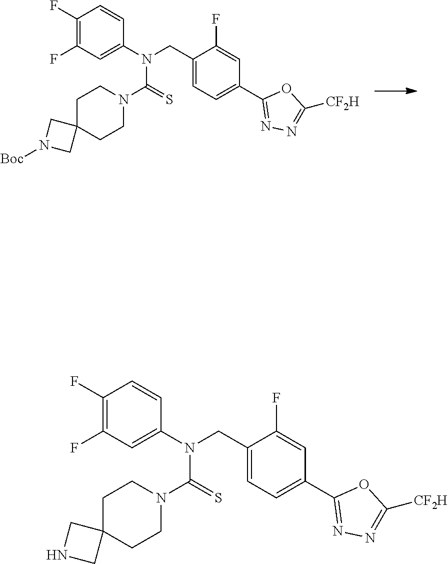 [SMILES: CC(C)(C)OC(=O)N1CC2(CCN(C(=S)N(Cc3ccc(-c4nnc(C(F)F)o4)cc3F)c3ccc(F)c(F)c3)CC2)C1 ; Fc1ccc(N(Cc2ccc(-c3nnc(C(F)F)o3)cc2F)C(=S)N2CCC3(CC2)CNC3)cc1F]

Tert-butyl 7-((4-(5-(difluoromethyl)-1,3,4-oxadiazol-2-yl)-2-fluorobenzyl)(3,4-difluorophenyl)carbamothioyl)-2,7-diazaspiro[3.5]nonan-2-carboxylate (0.600 g, 0.962 mmol) prepared in step 1 and trifluoroacetic acid (0.737 mL, 9.621 mmol) were dissolved in dichloromethane (10 mL) at room temperature, after which the resulting solution was stirred at the same temperature for 18 hours. Solvent was removed from the reaction mixture under reduced pressure, after which saturated aqueous sodium hydrogen carbonate solution was poured into the resulting concentrate and an organic layer was extracted with dichloromethane. The organic layer was washed with saturated aqueous sodium chloride solution, dehydrated with anhydrous sodium sulfate, filtered, and concentrated under reduced pressure. An obtained product was used without a further purification process (0.500 g, 99.3%, colorless oil).

### [Step 3] Synthesis of Compound 44

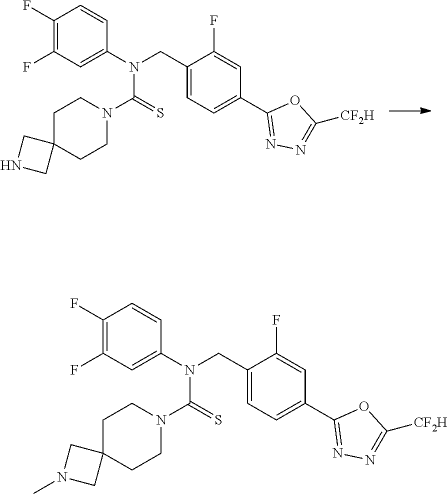 [SMILES: Fc1ccc(N(Cc2ccc(-c3nnc(C(F)F)o3)cc2F)C(=S)N2CCC3(CC2)CNC3)cc1F ; CN1CC2(CCN(C(=S)N(Cc3ccc(-c4nnc(C(F)F)o4)cc3F)c3ccc(F)c(F)c3)CC2)C1]

N-(4-(5-(difluoromethyl)-1,3,4-oxadiazol-2-yl)-2-fluorobenzyl)-N-(3,4-difluorophenyl)-2,7-diazaspiro[3.5]nonan-7-carbothioamide (0.216 g, 0.413 mmol) prepared in step 2, formaldehyde (0.025 g, 0.825 mmol) and N,N-diisopropylethylamine (0.144 mL, 0.825 mmol) were dissolved in dichloromethane (20 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.100 g, 45.1%) as a colorless oil form.

¹H NMR (400 MHz, CDCl₃) δ 7.87 (dd, J=8.0, 1.4 Hz, 1H), 7.81 (dd, J=10.3, 1.4 Hz, 1H), 7.73 (t, J=7.7 Hz, 1H), 7.14˜7.10 (m, 1H), 7.05 (s, 0.25H), 6.97˜6.93 (m, 1H), 6.93 (s, 0.5H), 6.85˜6.83 (m, 1H), 6.80 (s, 0.25H), 5.38 (s, 2H), 3.75˜3.55 (m, 4H), 3.36 (s, 4H), 2.56 (s, 3H), 1.72˜1.69 (m, 4H); LRMS (ES) m/z 538.7 (M⁺+1).

### Example 45: Synthesis of Compound 45, N-(4-(5-(difluoromethyl)-1,3,4-oxadiazol-2-yl)-2-fluorobenzyl)-N-(3,4-difluorophenyl)-2-(oxetan-3-yl)-2,7-diazaspiro[3.5]nonan-7-carbothioamide

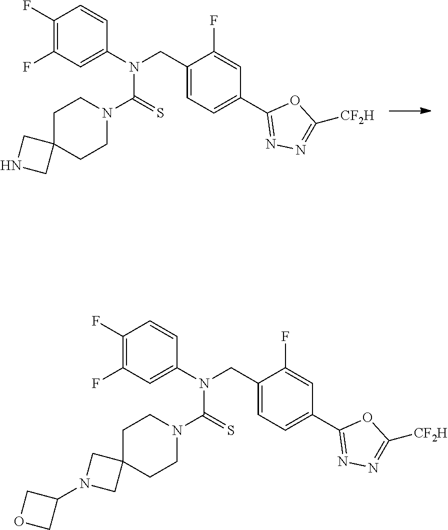 [SMILES: Fc1ccc(N(Cc2ccc(-c3nnc(C(F)F)o3)cc2F)C(=S)N2CCC3(CC2)CNC3)cc1F ; Fc1ccc(N(Cc2ccc(-c3nnc(C(F)F)o3)cc2F)C(=S)N2CCC3(CC2)CN(C2COC2)C3)cc1F]

N-(4-(5-(difluoromethyl)-1,3,4-oxadiazol-2-yl)-2-fluorobenzyl)-N-(3,4-difluorophenyl)-2,7-diazaspiro[3.5]nonan-7-carbothioamide (0.185 g, 0.353 mmol) prepared by the same method as described in step 2 of compound 44, 3-oxetanone (0.041 mL, 0.707 mmol) and N,N-diisopropylethylamine (0.123 mL, 0.707 mmol) were dissolved in dichloromethane (20 mL) at room temperature, after which the resulting solution was stirred at the same temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.035 g, 17.1%) as a colorless oil form.

¹H NMR (400 MHz, CDCl₃) δ 7.89˜7.87 (m, 1H), 7.82˜7.80 (m, 1H), 7.75˜7.71 (m, 1H), 7.17˜7.12 (m, 1H), 7.06 (s, 0.25H), 7.02˜6.94 (m, 1H), 6.93 (s, 0.5H), 6.89˜6.87 (m, 1H), 6.80 (s, 0.25H), 5.38 (s, 2H), 4.97˜4.93 (m, 2H), 4.70˜4.67 (m, 2H), 4.35˜4.25 (m, 1H), 3.80˜3.40 (m, 8H), 1.72˜1.69 (m, 4H); LRMS (ES) m/z 580.9 (M⁺+1).

### Example 46: Synthesis of Compound 46, N-(4-(5-(difluoromethyl)-1,3,4-oxadiazol-2-yl)-2-fluorobenzyl)-N-(3,4-difluorophenyl)-7-methyl-2,7-diazaspiro[3.5]nonan-2-carbothioamide

### [Step 1] Synthesis of tert-butyl 2-((4-(5-(difluoromethyl)-1,3,4-oxadiazol-2-yl)-2-fluorobenzyl)(3,4-difluorophenyl)carbamothioyl)-2,7-diazaspiro[3.5]nonan-7-carboxylate

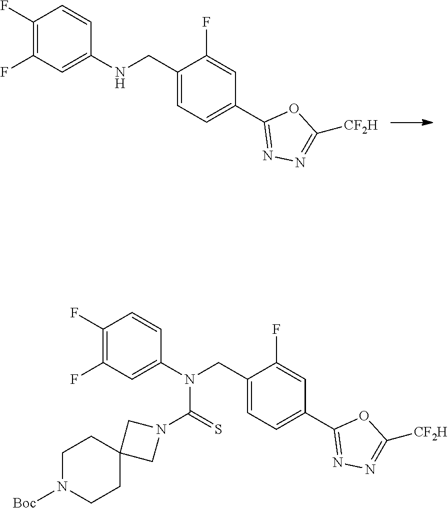 [SMILES: Fc1ccc(NCc2ccc(-c3nnc(C(F)F)o3)cc2F)cc1F ; CC(C)(C)OC(=O)N1CCC2(CC1)CN(C(=S)N(Cc1ccc(-c3nnc(C(F)F)o3)cc1F)c1ccc(F)c(F)c1)C2]

N-(4-(5-(difluoromethyl)-1,3,4-oxadiazol-2-yl)-2-fluorobenzyl)-3,4-difluoroaniline (1.000 g, 2.815 mmol) prepared by the same method as described in step 1 of compound 13, thiophosgene (0.216 mL, 2.815 mmol) and N,N-diisopropylethylamine (1.716 mL, 9.852 mmol) were dissolved in dichloromethane (30 mL), after which the resulting solution was stirred at ° C. for 30 minutes, added and further stirred at room temperature for 18 hours. Water was poured into the reaction mixture and an organic layer was extracted with dichloromethane. The organic layer was washed with saturated aqueous sodium chloride solution, dehydrated with anhydrous sodium sulfate, filtered, and concentrated under reduced pressure. The resulting concentrate was purified via column chromatography (SiO₂, 40 g cartridge; ethyl acetate/hexane=0 to 30%) and concentrated to obtain a title compound (0.230 g, 13.1%) as a yellow oil form.

### [Step 2] Synthesis of N-(4-(5-(difluoromethyl)-1,3,4-oxadiazol-2-yl)-2-fluorobenzyl)-N-(3,4-difluorophenyl)-2,7-diazaspiro[3.5]nonan-2-carbothioamide

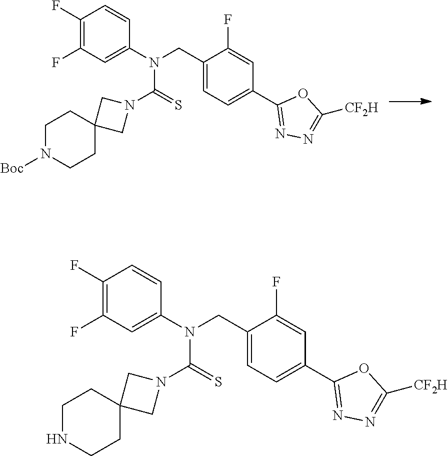 [SMILES: CC(C)(C)OC(=O)N1CCC2(CC1)CN(C(=S)N(Cc1ccc(-c3nnc(C(F)F)o3)cc1F)c1ccc(F)c(F)c1)C2 ; Fc1ccc(N(Cc2ccc(-c3nnc(C(F)F)o3)cc2F)C(=S)N2CC3(CCNCC3)C2)cc1F]

Tert-butyl 2-((4-(5-(difluoromethyl)-1,3,4-oxadiazol-2-yl)-2-fluorobenzyl)(3,4-difluorophenyl)carbamothioyl)-2,7-diazaspiro[3.5]nonan-7-carboxylate (0.230 g, 0.369 mmol) prepared in step land trifluoroacetic acid (0.282 mL, 3.688 mmol) were dissolved in dichloromethane (10 mL) at room temperature, after which the resulting solution was stirred at the same temperature for 18 hours. Solvent was removed from the reaction mixture under reduced pressure, after which saturated aqueous sodium hydrogen carbonate solution was poured into the resulting concentrate and an organic layer was extracted with dichloromethane. The organic layer was washed with saturated aqueous sodium chloride solution, dehydrated with anhydrous sodium sulfate, filtered, and concentrated under reduced pressure. An obtained product was used without a further purification process (0.150 g, 77.7%, colorless oil).

### [Step 3] Synthesis of Compound 46

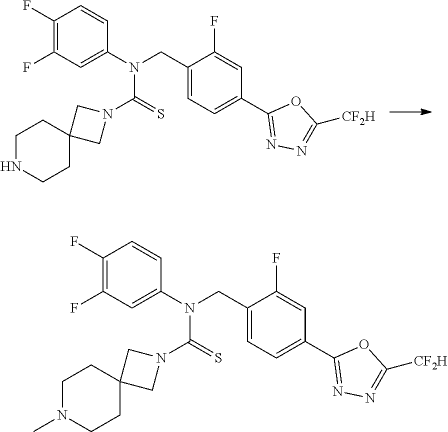 [SMILES: Fc1ccc(N(Cc2ccc(-c3nnc(C(F)F)o3)cc2F)C(=S)N2CC3(CCNCC3)C2)cc1F ; CN1CCC2(CC1)CN(C(=S)N(Cc1ccc(-c3nnc(C(F)F)o3)cc1F)c1ccc(F)c(F)c1)C2]

N-(4-(5-(difluoromethyl)-1,3,4-oxadiazol-2-yl)-2-fluorobenzyl)-N-(3,4-difluorophenyl)-2,7-diazaspiro[3.5]nonan-2-carbothioamide (0.139 g, 0.266 mmol) prepared in step 2, formaldehyde (0.016 g, 0.531 mmol) and N,N-diisopropylethylamine (0.092 mL, 0.531 mmol) were dissolved in dichloromethane (20 mL) at room temperature, after which the resulting solution was stirred at the same temperature for 18 hours. Water was poured into the reaction mixture and an organic layer was extracted with dichloromethane. The organic layer was washed with saturated sodium chloride aqueous solution, dehydrated with anhydrous sodium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.060 g, 42.0%) as a black oil form.

¹H NMR (400 MHz, CDCl₃) δ 7.95˜7.88 (m, 2H), 7.72 (dd, J=10.0, 1.4 Hz, 1H), 7.13 (dd, J=18.2, 8.8 Hz, 1H), 7.05 (s, 0.25H), 7.02˜6.97 (m, 1H), 6.92 (s, 0.5H), 6.90˜6.87 (m, 1H), 6.79 (s, 0.25H), 5.57 (s, 2H), 3.80˜3.20 (m, 4H), 2.60˜2.40 (m, 4H), 2.32 (s, 3H), 1.74 (t, J=5.4 Hz, 4H); LRMS (ES) m/z 538.7 (M⁺+1).

### Protocol for Measuring and Analyzing Activity of Compound of Present Invention

### Experimental Example 1. Confirmation of HDAC Enzyme Activity Inhibition (In Vitro)

### 1. Experimental Method

An HDAC enzyme inhibitory capacity of test material was measured by using HDAC1 Fluorimetric Drug Discovery Assay Kit (Enzolifesciences: BML-AK511) and HDAC6 human recombinant (Calbiochem: 382180). For a HDAC1 assay, samples were treated at a concentration of 100 nM, 1000 nM and 10000 nM. For an HDAC6 assay, samples were treated at a concentration of 0.1 nM, 1 nM, 10 nM, 100 nM and 1000 nM. After the sample treatment, a reaction was continued at 37° C. for 60 minutes, treated with a developer, and subjected to reaction at 37° C. for 30 minutes, after which fluorescence intensity (Ex 390 nm, Em 460 nm) was measured by using FlexStation3 (Molecular device). For final result values, each IC₅₁ value was calculated with GraphPad Prism 4.0 program.

### 2. Experimental Results

The results of searching HDAC enzyme activity inhibition obtained according to the experimental method are shown in table 2.

TABLE 2

| | HDAC6 | HDAC1 |
|---|---|---|
| Compound | IC₅₀ (uM) | IC₅₀ (uM) |
| 1 | 0.086 | >10 |
| 2 | 0.066 | >10 |
| 3 | 0.037 | >10 |
| 4 | 0.037 | >10 |
| 5 | 0.037 | >10 |
| 6 | 0.031 | >10 |
| 7 | 0.036 | >10 |
| 8 | 0.075 | >10 |
| 9 | 0.138 | >10 |
| 10 | 0.045 | >10 |
| 11 | 0.050 | >10 |
| 12 | 0.135 | >10 |
| 13 | 0.076 | >10 |
| 14 | 0.039 | >10 |
| 15 | 0.054 | >10 |
| 16 | 0.063 | >10 |
| 17 | 0.041 | >10 |
| 18 | 0.061 | >10 |
| 19 | 0.054 | >10 |
| 20 | 0.060 | >10 |
| 21 | 0.070 | >10 |
| 22 | 0.083 | >10 |
| 23 | 0.062 | >10 |
| 24 | 0.083 | >10 |
| 25 | 0.046 | >10 |
| 26 | 0.089 | >10 |
| 27 | 0.112 | >10 |
| 28 | 0.077 | >10 |
| 29 | 0.070 | >10 |
| 30 | 0.024 | >10 |
| 31 | 0.035 | >10 |
| 32 | 0.045 | >10 |
| 33 | 0.035 | >10 |
| 34 | 0.047 | >10 |
| 35 | 0.066 | >10 |
| 36 | 0.043 | >10 |
| 37 | 0.067 | >10 |
| 38 | 0.051 | >10 |
| 39 | 0.030 | >10 |
| 40 | 0.055 | >10 |
| 41 | 0.073 | >10 |
| 42 | 0.057 | >10 |
| 43 | 0.077 | >10 |
| 44 | 0.080 | >10 |
| 45 | 0.196 | >10 |
| 46 | 0.028 | >10 |

As described in table 2, it was confirmed from the results of testing the activity inhibition to HDAC1 and HDAC6 that the thiocarbonyl compound of the present invention, stereoisomers thereof, or pharmaceutically acceptable salts thereof show an excellent selective HDAC6 inhibitory activity to HDAC1.

1. A 1,3,4-oxadiazole thiocarbonyl compound represented by formula I, stereoisomers thereof or pharmaceutically acceptable salts thereof: 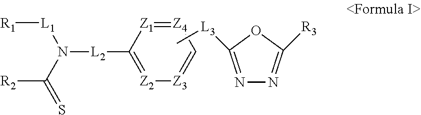
wherein,
L₁, L₂ and L₃ are each independently a single bond or —(C₁-C₄ alkylene)-;
R₁ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl, 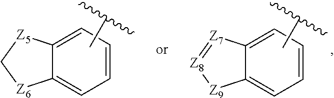
in R¹,
at least one H of —(C₁-C₄ alkyl) may be substituted with -T or —OH,
at least one H of -aryl or -heteroaryl may be each independently substituted with -T, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), -aryl, -heteroaryl, 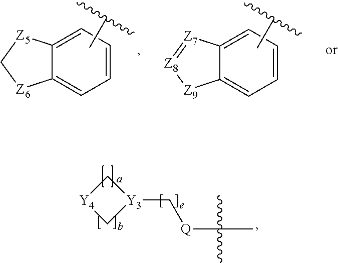
in which at least one H of 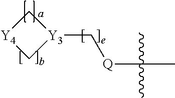
may be substituted with -T, —(C₁-C₄ alkyl), —CF₃ or —CF₂H,
at least one H of —(C₃-C₇ cycloalkyl), —(C₂-C₆ cycloheteroalkyl), -adamantyl, 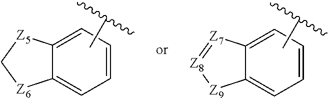
may be each independently substituted with -T, —OH or —(C₁-C₄ alkyl);
R₂ is —NR^{A}R^{B}, —OR^{C}, -heteroaryl, 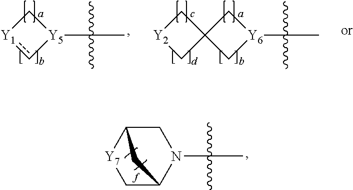
in R₂,
at least one H of 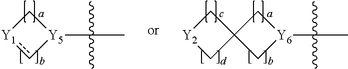
may be substituted with -T, —OH, —O(C₁-C₄ alkyl), —NR^{D}R^{E}, —(C₁-C₄ alkyl), —CF₃, —CF₂H, —CN, -aryl, -heteroaryl, —(C₁-C₄ alkyl)-aryl or —(C₁-C₄ alkyl)-heteroaryl, in which at least one H of -aryl, -heteroaryl, —(C₁-C₄ alkyl)-aryl or —(C₁-C₄ alkyl)-heteroaryl may be substituted with -T, —OH, —CF₃ or —CF₂H;
R₃ is -CT₃ or -CT₂H;
Y₁, Y₂, Y₄ and Y₇ are each independently ═CH—, —CHR^{F}—, —NR^{F}—, —O—, —C(═O)— or S(═O)₂—;
Y₃, Y₅ and Y₆ are each independently —CH— or —N—;
Z₁ to Z₄ are each independently N or CR^{Z};
in Z₁ to Z₄,
at least three of Z₁ to Z₄ may not be N at the same time, and R^{Z} is —H, -T or —O(C₁-C₄ alkyl);
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ alkyl)-heteroaryl, —(C₃-C₇ cycloalkyl), —(C₂-C₆ heterocycloalkyl) or 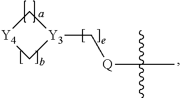
in R^{A} and R^{B},
at least one H of —(C₁-C₄ alkyl), —(C₁-C₄ alkyl)-OH or —(C₁-C₄ alkyl)-NR^{D}R^{E} may be substituted with -T,
at least one H of -aryl, —(C₁-C₄ alkyl)-aryl, -heteroaryl, —(C₁-C₄ alkyl)-heteroaryl, —(C₃-C₇ cycloalkyl) or —(C₂-C₆ heterocycloalkyl) may be substituted with -T, —OH, —O(C₁-C₄ alkyl), —(C₁-C₄ alkyl), —CF₃, —CF₂H or —CN,
at least one H of 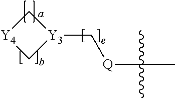
may be substituted with -T, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl or -heteroaryl;
R^{C} is —(C₁-C₄ alkyl), -aryl, —(C₁-C₄ alkyl)-aryl, -heteroaryl or —(C₁-C₄ alkyl)-heteroaryl, in R^{C},
at least one H of —(C₁-C₄ alkyl) may be substituted with -T or —OH,
at least one H of -aryl, —(C₁-C₄ alkyl)-aryl, -heteroaryl or —(C₁-C₄ alkyl)-heteroaryl may be substituted with -T, —OH, —CF₃ or —CF₂H;
R^{D} and R^{E} are each independently —H, —(C₁-C₄ alkyl), -aryl or —(C₁-C₄ alkyl)-aryl, in R^{D} and R^{E},
at least one H of —(C₁-C₄ alkyl) may be substituted with -T or —OH, at least one H of -aryl or —(C₁-C₄ alkyl)-aryl may be substituted with -T, —OH, —CF₃ or —CF₂H;
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —NR^{D}R^{E}, —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl),
in R^{F},
at least one H of —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —NR^{D}R^{E}, —(C₁-C₄ alkyl)-NR^{D}R^{E} or —S(═O)₂—(C₁-C₄ alkyl) may be substituted with -T,
at least one H of -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl) may be substituted with -T, —OH, —(C₁-C₄ alkyl), —CF₃ or —CF₂H;
R^{G} is —H or —(C₁-C₄ alkyl);
Q is —O— or a single bond; [symbol]
is a single bond or a double bond, provided that when [symbol] is a double bond, Y₁ is ═CH—;
a to e are each independently an integer of 0, 1, 2, 3 or 4, provided that a and b may not be 0 together, and c and d may not be 0 together;
f is an integer of 1 or 2; and
T is F, Cl, Br or I.

2. The 1,3,4-oxadiazole thiocarbonyl compound represented by formula I, stereoisomers thereof or pharmaceutically acceptable salts thereof according to claim 1, wherein in formula I,
L₁, L₂ and L₃ are each independently a single bond or —(C₁-C₂ alkylene)-;
R¹ is —(C₁-C₄ alkyl), —(C₆-C₁₂ aryl) or —(C₃-C₁₀ heteroaryl) including at least one heteroatom selected from the group consisting of O, N and S,
in R¹,
at least one H of —(C₁-C₄ alkyl) may be substituted with -T or —OH,
at least one H of —(C₆-C₁₂ aryl) or —(C₃-C₁₀ heteroaryl) including at least one heteroatom selected from the group consisting of O, N and S may be each independently substituted with -T, —CF₃ or —CF₂H;
R₂ is —(C₃-C₁₀ heteroaryl) including at least one heteroatom selected from the group consisting of O, N and S, b, d b or; 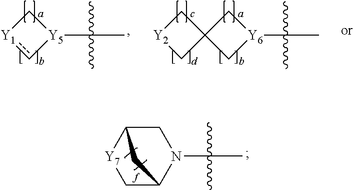
R₃ is -CT₃ or -CT₂H;
Y₁, Y₂, Y₄ and Y₇ are each independently ═CH—, —CHR^{F}—, —NR^{F}—, —O—, —C(═O)— or S(═O)₂—;
Y₃, Y₅ and Y₆ are each independently —CH— or —N—;
Z₁ to Z₄ are each independently N or CR^{Z},
in Z₁ to Z₄,
at least three of Z₁ to Z₄ may not be N at the same time,
R^{z} is —H, -T or —O(C₁-C₄ alkyl);
R^{F} is —H, —(C₁-C₆ alkyl), —C(═O)—(C₁-C₄ alkyl) or —(C₂-C₆ heterocycloalkyl); [symbol]
is a single bond or a double bond, provided that when [symbol] is a double bond, Y₁ is ═CH—;
a to e are each independently an integer of 0, 1, 2, 3 or 4, provided that a and b may not be 0 together, and c and d may not be 0 together;
f is an integer of 1 or 2; and
T is F, Cl, Br or I.

3. The 1,3,4-oxadiazole thiocarbonyl compound represented by formula I, stereoisomers thereof or pharmaceutically acceptable salts thereof according to claim 1, wherein the compound represented by formula I is any one selected from the group consisting of compounds 1 to 46;

| Compound | Structure |
|---|---|
| 1 | 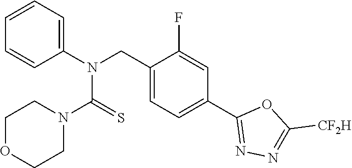 [SMILES: Fc1cc(-c2nnc(C(F)F)o2)ccc1CN(C(=S)N1CCOCC1)c1ccccc1] |
| 2 | 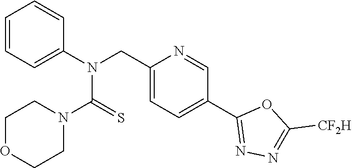 [SMILES: FC(F)c1nnc(-c2ccc(CN(C(=S)N3CCOCC3)c3ccccc3)nc2)o1] |
| 3 | 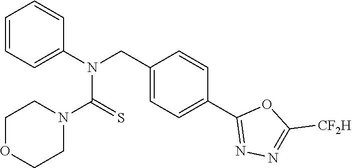 [SMILES: FC(F)c1nnc(-c2ccc(CN(C(=S)N3CCOCC3)c3ccccc3)cc2)o1] |
| 4 | 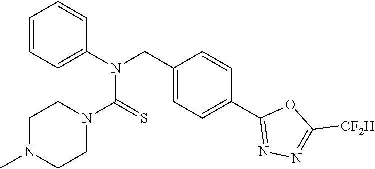 [SMILES: CN1CCN(C(=S)N(Cc2ccc(-c3nnc(C(F)F)o3)cc2)c2ccccc2)CC1] |
| 5 | 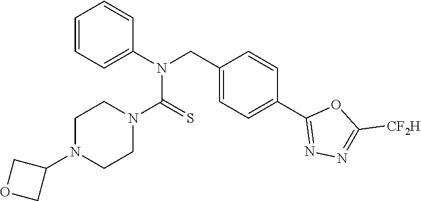 [SMILES: FC(F)c1nnc(-c2ccc(CN(C(=S)N3CCN(C4COC4)CC3)c3ccccc3)cc2)o1] |
| 6 | 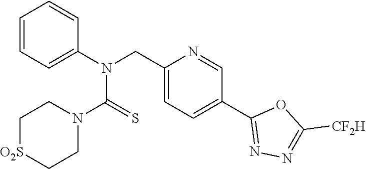 [SMILES: O=S1(=O)CCN(C(=S)N(Cc2ccc(-c3nnc(C(F)F)o3)cn2)c2ccccc2)CC1] |
| 7 | 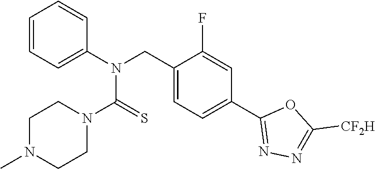 [SMILES: CN1CCN(C(=S)N(Cc2ccc(-c3nnc(C(F)F)o3)cc2F)c2ccccc2)CC1] |
| 8 | 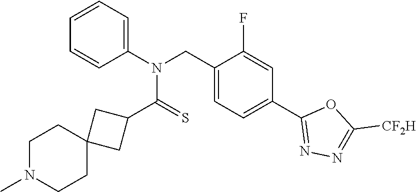 [SMILES: CN1CCC2(CC1)CC(C(=S)N(Cc1ccc(-c3nnc(C(F)F)o3)cc1F)c1ccccc1)C2] |
| 9 | 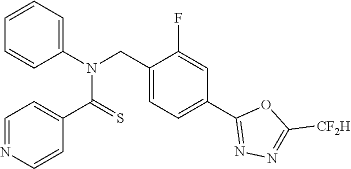 [SMILES: Fc1cc(-c2nnc(C(F)F)o2)ccc1CN(C(=S)c1ccncc1)c1ccccc1] |
| 10 | 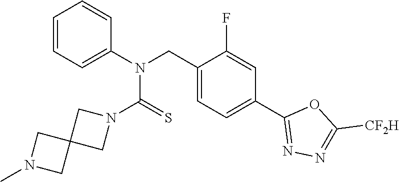 [SMILES: CN1CC2(C1)CN(C(=S)N(Cc1ccc(-c3nnc(C(F)F)o3)cc1F)c1ccccc1)C2] |
| 11 | 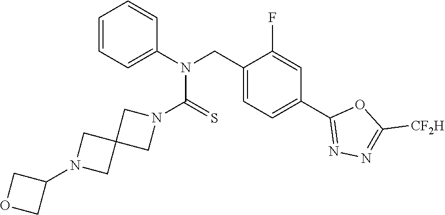 [SMILES: Fc1cc(-c2nnc(C(F)F)o2)ccc1CN(C(=S)N1CC2(C1)CN(C1COC1)C2)c1ccccc1] |
| 12 | 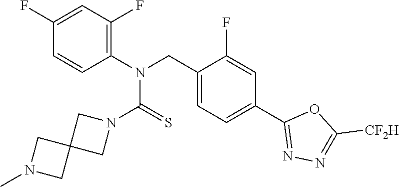 [SMILES: CN1CC2(C1)CN(C(=S)N(Cc1ccc(-c3nnc(C(F)F)o3)cc1F)c1ccc(F)cc1F)C2] |
| 13 | 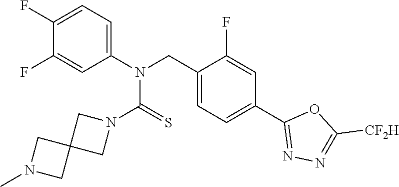 [SMILES: CN1CC2(C1)CN(C(=S)N(Cc1ccc(-c3nnc(C(F)F)o3)cc1F)c1ccc(F)c(F)c1)C2] |
| 14 | 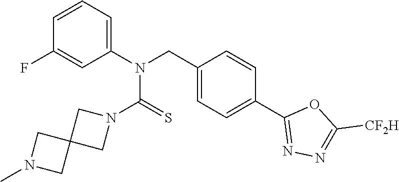 [SMILES: CN1CC2(C1)CN(C(=S)N(Cc1ccc(-c3nnc(C(F)F)o3)cc1)c1cccc(F)c1)C2] |
| 15 | 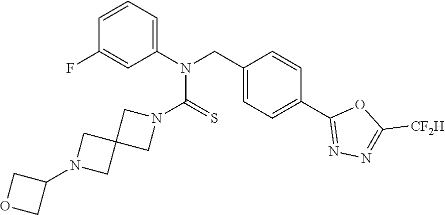 [SMILES: Fc1cccc(N(Cc2ccc(-c3nnc(C(F)F)o3)cc2)C(=S)N2CC3(C2)CN(C2COC2)C3)c1] |
| 16 | 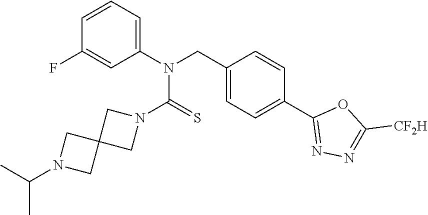 [SMILES: CC(C)N1CC2(CN(C(=S)N(Cc3ccc(-c4nnc(C(F)F)o4)cc3)c3cccc(F)c3)C2)C1] |
| 17 | 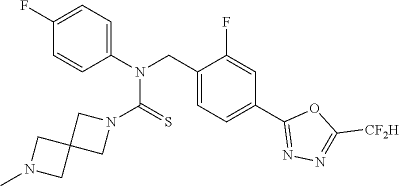 [SMILES: CN1CC2(C1)CN(C(=S)N(Cc1ccc(-c3nnc(C(F)F)o3)cc1F)c1ccc(F)cc1)C2] |
| 18 | 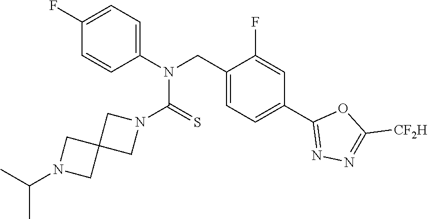 [SMILES: CC(C)N1CC2(CN(C(=S)N(Cc3ccc(-c4nnc(C(F)F)o4)cc3F)c3ccc(F)cc3)C2)C1] |
| 19 | 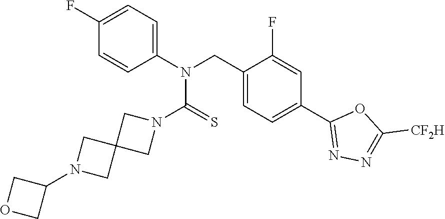 [SMILES: Fc1ccc(N(Cc2ccc(-c3nnc(C(F)F)o3)cc2F)C(=S)N2CC3(C2)CN(C2COC2)C3)cc1] |
| 20 | 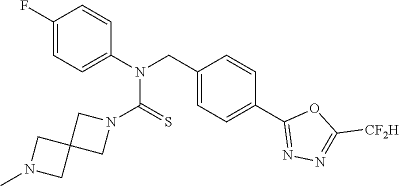 [SMILES: CN1CC2(C1)CN(C(=S)N(Cc1ccc(-c3nnc(C(F)F)o3)cc1)c1ccc(F)cc1)C2] |
| 21 | 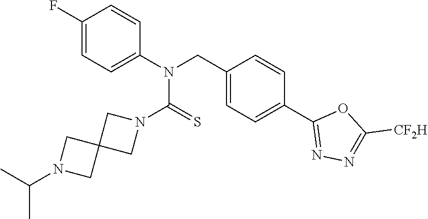 [SMILES: CC(C)N1CC2(CN(C(=S)N(Cc3ccc(-c4nnc(C(F)F)o4)cc3)c3ccc(F)cc3)C2)C1] |
| 22 | 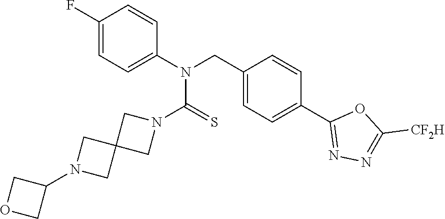 [SMILES: Fc1ccc(N(Cc2ccc(-c3nnc(C(F)F)o3)cc2)C(=S)N2CC3(C2)CN(C2COC2)C3)cc1] |
| 23 | 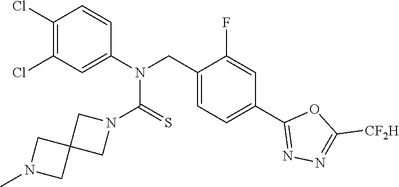 [SMILES: CN1CC2(C1)CN(C(=S)N(Cc1ccc(-c3nnc(C(F)F)o3)cc1F)c1ccc(Cl)c(Cl)c1)C2] |
| 24 | 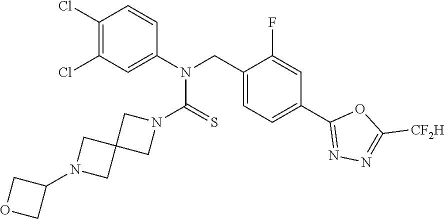 [SMILES: Fc1cc(-c2nnc(C(F)F)o2)ccc1CN(C(=S)N1CC2(C1)CN(C1COC1)C2)c1ccc(Cl)c(Cl)c1] |
| 25 | 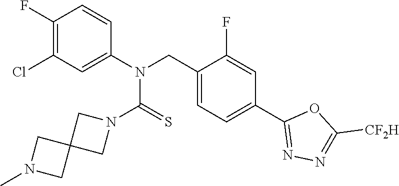 [SMILES: CN1CC2(C1)CN(C(=S)N(Cc1ccc(-c3nnc(C(F)F)o3)cc1F)c1ccc(F)c(Cl)c1)C2] |
| 26 | 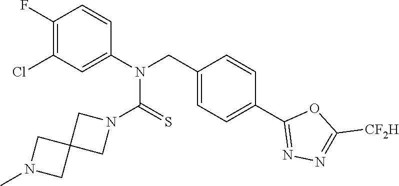 [SMILES: CN1CC2(C1)CN(C(=S)N(Cc1ccc(-c3nnc(C(F)F)o3)cc1)c1ccc(F)c(Cl)c1)C2] |
| 27 | 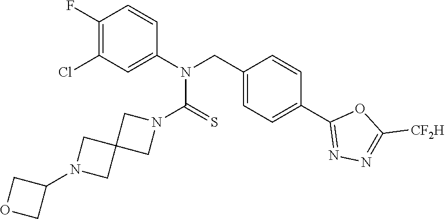 [SMILES: Fc1ccc(N(Cc2ccc(-c3nnc(C(F)F)o3)cc2)C(=S)N2CC3(C2)CN(C2COC2)C3)cc1Cl] |
| 28 | 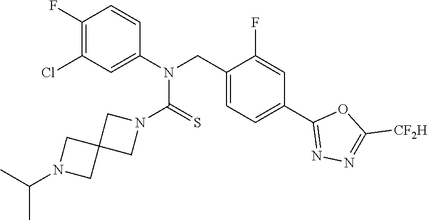 [SMILES: CC(C)N1CC2(CN(C(=S)N(Cc3ccc(-c4nnc(C(F)F)o4)cc3F)c3ccc(F)c(Cl)c3)C2)C1] |
| 29 | 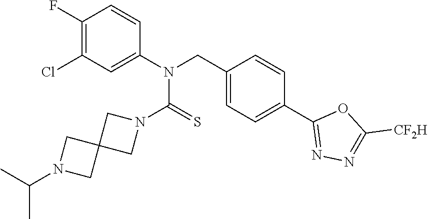 [SMILES: CC(C)N1CC2(CN(C(=S)N(Cc3ccc(-c4nnc(C(F)F)o4)cc3)c3ccc(F)c(Cl)c3)C2)C1] |
| 30 | 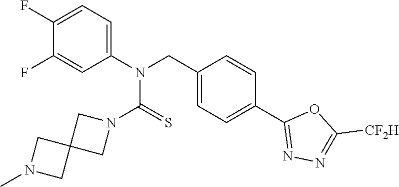 [SMILES: CN1CC2(C1)CN(C(=S)N(Cc1ccc(-c3nnc(C(F)F)o3)cc1)c1ccc(F)c(F)c1)C2] |
| 31 | 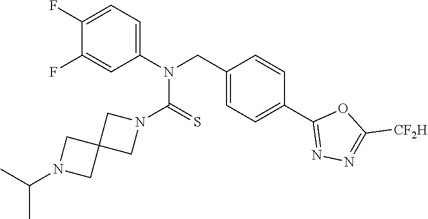 [SMILES: CC(C)N1CC2(CN(C(=S)N(Cc3ccc(-c4nnc(C(F)F)o4)cc3)c3ccc(F)c(F)c3)C2)C1] |
| 32 | 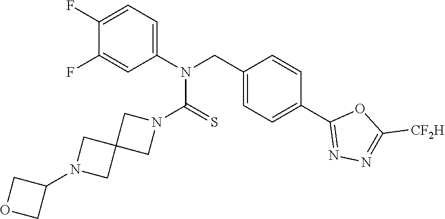 [SMILES: Fc1ccc(N(Cc2ccc(-c3nnc(C(F)F)o3)cc2)C(=S)N2CC3(C2)CN(C2COC2)C3)cc1F] |
| 33 | 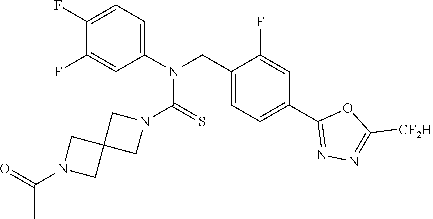 [SMILES: CC(=O)N1CC2(C1)CN(C(=S)N(Cc1ccc(-c3nnc(C(F)F)o3)cc1F)c1ccc(F)c(F)c1)C2] |
| 34 | 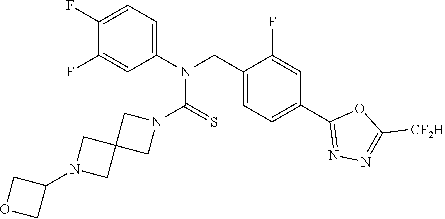 [SMILES: Fc1ccc(N(Cc2ccc(-c3nnc(C(F)F)o3)cc2F)C(=S)N2CC3(C2)CN(C2COC2)C3)cc1F] |
| 35 | 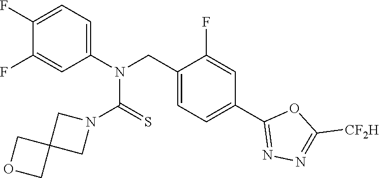 [SMILES: Fc1ccc(N(Cc2ccc(-c3nnc(C(F)F)o3)cc2F)C(=S)N2CC3(COC3)C2)cc1F] |
| 36 | 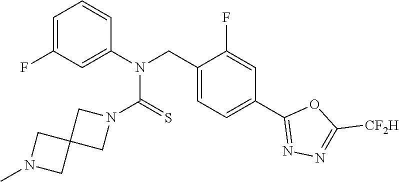 [SMILES: CN1CC2(C1)CN(C(=S)N(Cc1ccc(-c3nnc(C(F)F)o3)cc1F)c1cccc(F)c1)C2] |
| 37 | 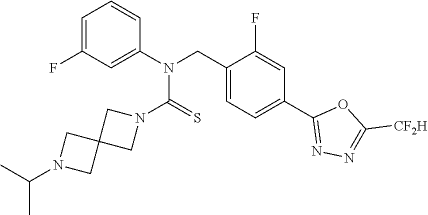 [SMILES: CC(C)N1CC2(CN(C(=S)N(Cc3ccc(-c4nnc(C(F)F)o4)cc3F)c3cccc(F)c3)C2)C1] |
| 38 | 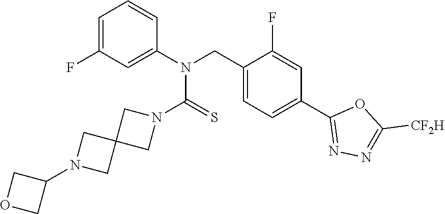 [SMILES: Fc1cccc(N(Cc2ccc(-c3nnc(C(F)F)o3)cc2F)C(=S)N2CC3(C2)CN(C2COC2)C3)c1] |
| 39 | 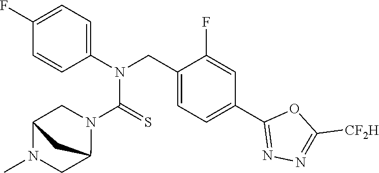 [SMILES: CN1C[C@@H]2C[C@H]1CN2C(=S)N(Cc1ccc(-c2nnc(C(F)F)o2)cc1F)c1ccc(F)cc1] |
| 40 | 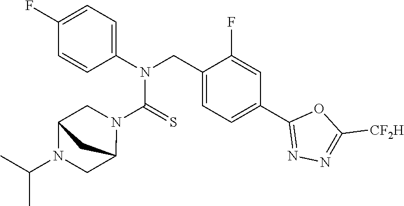 [SMILES: CC(C)N1C[C@@H]2C[C@H]1CN2C(=S)N(Cc1ccc(-c2nnc(C(F)F)o2)cc1F)c1ccc(F)cc1] |
| 41 | 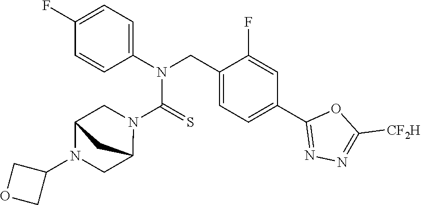 [SMILES: Fc1ccc(N(Cc2ccc(-c3nnc(C(F)F)o3)cc2F)C(=S)N2C[C@@H]3C[C@H]2CN3C2COC2)cc1] |
| 42 | 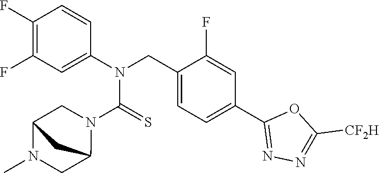 [SMILES: CN1C[C@@H]2C[C@H]1CN2C(=S)N(Cc1ccc(-c2nnc(C(F)F)o2)cc1F)c1ccc(F)c(F)c1] |
| 43 | 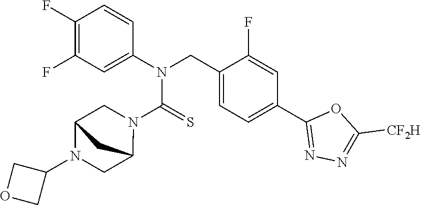 [SMILES: Fc1ccc(N(Cc2ccc(-c3nnc(C(F)F)o3)cc2F)C(=S)N2C[C@@H]3C[C@H]2CN3C2COC2)cc1F] |
| 44 | 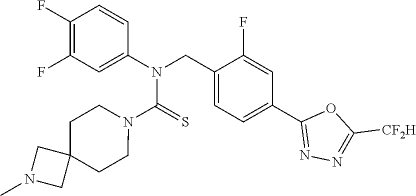 [SMILES: CN1CC2(CCN(C(=S)N(Cc3ccc(-c4nnc(C(F)F)o4)cc3F)c3ccc(F)c(F)c3)CC2)C1] |
| 45 | 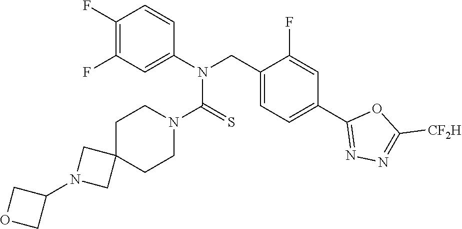 [SMILES: Fc1ccc(N(Cc2ccc(-c3nnc(C(F)F)o3)cc2F)C(=S)N2CCC3(CC2)CN(C2COC2)C3)cc1F] |
| 46 | 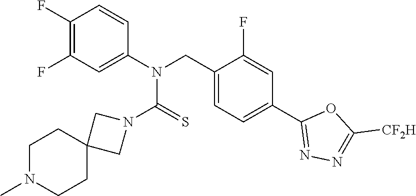 [SMILES: CN1CCC2(CC1)CN(C(=S)N(Cc1ccc(-c3nnc(C(F)F)o3)cc1F)c1ccc(F)c(F)c1)C2] |

4. A pharmaceutical composition comprising the 1,3,4-oxadiazole thiocarbonyl compound according to claim 1, stereoisomers thereof or pharmaceutically acceptable salts thereof as an active ingredient.

5. The pharmaceutical composition according to claim 4, wherein the pharmaceutical composition is for the prevention or treatment of histone deacetylase (HDAC)-mediated diseases.

6. The pharmaceutical composition according to claim 5, wherein the histone deacetylase (HDAC)-mediated diseases are infectious diseases; neoplasm; endocrinopathy, nutritional and metabolic diseases; mental and behavioral disorders; neurological diseases; eye and ocular adnexal diseases; circulatory diseases; respiratory diseases; digestive troubles; skin and subcutaneous tissue diseases; musculoskeletal system and connective tissue diseases; or teratosis, deformities and chromosomal aberration.

7. The pharmaceutical composition according to claim 6,
wherein
the infectious diseases are prion disease;
the neoplasm is benign tumor or malignant tumor;
the endocrinopathy, nutritional and metabolic diseases are Wilson's disease, amyloidosis or diabetes;
the mental and behavioral disorders are depression or rett syndrome;
the neurological diseases are central nervous system atrophy, neurodegenerative disease, motor disorder, neuropathy, motor neuron disease or central nervous system demyelinating disease;
the eye and ocular adnexal diseases are uveitis;
the skin and subcutaneous tissue diseases are psoriasis; the circulatory diseases are atrial fibrillation or stroke; the respiratory diseases are asthma;
the digestive troubles are alcoholic liver disease, inflammatory bowel disease, Crohn's disease or ulcerative bowel disease;
the musculoskeletal system and connective tissue diseases are rheumatoid arthritis, osteoarthritis or systemic lupus erythematosis; and
the teratosis, deformities and chromosomal aberration are autosomal dominant polycystic kidney disease.

8. A method for preventing or treating histone deacetylase (HDAC)-mediated diseases, the method comprising administering a therapeutically effective amount of the 1,3,4-oxadiazole thiocarbonyl compound according to claim 1, stereoisomers thereof or pharmaceutically acceptable salts thereof.

9-10. (canceled)
